(19) United States
(12) Patent Application Publication
(10) Pub. No.: US 2014/0248215 A1
(43) Pub. Date: Sep. 4, 2014

(54) USES AND COMPOSITIONS FOR TREATMENT OF PSORIATIC ARTHRITIS

(71) Applicants: Rebecca S. Hoffman, Wilmette, IL (US); Eric H. Sasso, Evanston, IL (US); Renee J. Perdok, Gurnee, IL (US); Philip Yan, Vernon Hills, IL (US); Mark Weinberg, Glenview, IL (US); Phillip Mease, Seattle, WA (US); Christopher T. Ritchlin, Canandaigua, NY (US); Dafna D. Gladman, Toronto (CA)

(72) Inventors: Rebecca S. Hoffman, Wilmette, IL (US); Eric H. Sasso, Evanston, IL (US); Renee J. Perdok, Gurnee, IL (US); Philip Yan, Vernon Hills, IL (US); Mark Weinberg, Glenview, IL (US); Phillip Mease, Seattle, WA (US); Christopher T. Ritchlin, Canandaigua, NY (US); Dafna D. Gladman, Toronto (CA)

(21) Appl. No.: 14/010,172

(22) Filed: Aug. 26, 2013

Related U.S. Application Data
(63) Continuation of application No. 11/811,141, filed on Jun. 8, 2007, now abandoned.
(60) Provisional application No. 60/812,312, filed on Jun. 8, 2006.
(60) Provisional application No. 60/832,370, filed on Jul. 20, 2006.
(60) Provisional application No. 60/851,830, filed on Oct. 12, 2006.
(60) Provisional application No. 60/858,328, filed on Nov. 10, 2006.

(51) Int. Cl. C07K 16/24; A61K 49/00

(52) U.S. Cl. CPC C07K 16/241; A61K 49/0004
USPC 424/9.2; 424/142.1

(57) ABSTRACT

The invention provides methods, uses and compositions for the treatment of psoriatic arthritis. The invention describes methods and uses for treating psoriatic arthritis, wherein a TNFα inhibitor, such as a human TNFα antibody, or antigen-binding portion thereof, is used to psoriatic arthritis in a subject. Also described are methods for determining the efficacy of a TNFα inhibitor for treatment of psoriatic arthritis in a subject.

### RELATED APPLICATIONS

This application is a continuation of U.S. patent application Ser. No. 11/811,141, filed Jun. 8, 2007; which claims priority to U.S. Provisional Patent Application No. 60/812,312, filed on Jun. 8, 2006; U.S. Provisional Patent Application No. 60/832,370, filed on Jul. 20, 2006; U.S. Provisional Patent Application No. 60/851,830, filed on Oct. 12, 2006; and U.S. Provisional Patent Application No. 60/858,328, filed on Nov. 10, 2006. The contents of the aforementioned applications are hereby incorporated by reference.

### BACKGROUND

Psoriatic arthritis (or PsA) is an inflammatory condition that affects the joints of children and adults with psoriasis. Psoriasis is a skin condition that causes patches of thick, red skin to form on certain areas of your body. Psoriatic arthritis may affect one joint or many. Signs and symptoms of psoriatic arthritis include pain in affected joints, swollen joints, and joints that are warm to the touch. Psoriatic arthritis can be debilitating and painful, making it difficult for those affected to perform even daily routines. Despite medications, psoriatic arthritis can also cause erosion in joints of patients having PsA.

No cure exists for psoriatic arthritis. Generally, treatment includes trying to control inflammation in affected joints in order to prevent joint pain and disability. Medications commonly used to treat psoriatic arthritis include: Nonsteroidal anti-inflammatory drugs (NSAIDs), corticosteroids, and disease-modifying antirheumatic drugs (DMARDs).

Tumor necrosis factor has been implicated in the pathophysiology of psoriatic arthritis (Partsch et al. (1998) Ann Rheum Dis. 57:691; Ritchlin et al. (1998) J Rheumatol. 25:1544). In recent years biologic response modifiers that inhibit TNF activity have become established therapies for PsA. Adalimumab, etanercept, and infliximab have demonstrated improvements in treating subjects having PsA.

### SUMMARY OF THE INVENTION

Although TNFα inhibitors are effective at treating PsA, there remains a need for a more effective treatment option for subjects suffering from psoriatic arthritis (PsA), especially in improving the quality of life of subjects having PsA, methods of inhibiting radiographic progression associated with PsA, and certain subpopulations of PsA patients. There also remains a need for improved methods and compositions that provide a safe and effective treatment of PsA using TNFα inhibitors.

The instant invention provides improved methods and compositions for treating PsA. The invention further provides a means for treating certain subpopulations of patients who have PsA, including subjects or patients who have failed therapy or lost responsiveness to treatment with TNFα inhibitors. The invention further provides a means by which the efficacy of a TNFα inhibitor for the treatment of PsA can be determined Each of the examples described herein describes methods and compositions which can be used to determine whether a TNFα inhibitor is effective for treating the given disorder, i.e. PsA.

The invention also provides a method of determining the efficacy of a TNFα inhibitor for treating psoriatic arthritis in a subject comprising: determining an ACR20 response of a patient population having psoriatic arthritis who was administered the TNFα inhibitor, wherein an ACR20 response in at least about 39% of the patient population indicates that the TNFα inhibitor is an effective TNFα inhibitor for the treatment of psoriatic arthritis.

In one embodiment, the invention further comprises administering the effective TNFα inhibitor to a subject to treat psoriatic arthritis. In another embodiment, an ACR20 response in at least about 45% of the patient population indicates that the TNFα inhibitor is an effective TNFα inhibitor for the treatment of psoriatic arthritis. In yet another embodiment, an ACR20 response in at least about 50% of the patient population indicates that the TNFα inhibitor is an effective TNFα inhibitor for the treatment of psoriatic arthritis. In another embodiment, an ACR20 response in at least about 55% of the patient population indicates that the TNFα inhibitor is an effective TNFα inhibitor for the treatment of psoriatic arthritis. In another embodiment, an ACR20 response in at least about 61% of the patient population indicates that the TNFα inhibitor is an effective TNFα inhibitor for the treatment of psoriatic arthritis.

The invention further provides a method of treating psoriatic arthritis in a subject comprising administering an effective TNFα inhibitor to the subject such that psoriatic arthritis is treated, wherein the effective TNFα inhibitor was previously identified as achieving an ACR20 response in at least about 39% of a patient population having PsA. In one embodiment, the effective TNFα inhibitor was previously identified as achieving an ACR20 response in at least about 45% of the patient population. In another embodiment, the effective TNFα inhibitor was previously identified as achieving an ACR20 response in at least about 50% of the patient population. In another embodiment, the effective TNFα inhibitor was previously identified as achieving an ACR20 response in at least about 55% of the patient population. In yet another embodiment, the effective TNFα inhibitor was previously identified as achieving an ACR50 response in at least about 61% of the patient population.

The invention further provides a method of determining the efficacy of a TNFα inhibitor for treating psoriatic arthritis in a subject comprising: determining an ACR50 response of a patient population having psoriatic arthritis who was administered the TNFα inhibitor, wherein an ACR50 response in at least about 25% of the patient population indicates that the TNFα inhibitor is an effective TNFα inhibitor for the treatment of psoriatic arthritis. In one embodiment, the invention further comprises administering the effective TNFα inhibitor to a subject to treat psoriatic arthritis. In one embodiment, an ACR50 response in at least about 30% of the patient population indicates that the TNFα inhibitor is an effective TNFα inhibitor for the treatment of psoriatic arthritis in a subject. In another embodiment, an ACR50 response in at least about 35% of the patient population indicates that the TNFα inhibitor is an effective TNFα inhibitor for the treatment of psoriatic arthritis in a subject. In another embodiment, an ACR50 response in at least about 40% of the patient population indicates that the TNFα inhibitor is an effective TNFα inhibitor for the treatment of psoriatic arthritis in a subject. In another embodiment, an ACR50 response in at least about 46% of the patient population indicates that TNFα inhibitor is an effective TNFα inhibitor for the treatment of psoriatic arthritis in a subject.

The invention also provides a method of treating psoriatic arthritis in a subject comprising administering an effective TNFα inhibitor to the subject such that psoriatic arthritis is treated, wherein the effective TNFα inhibitor was previously identified as achieving an ACR50 response in at least about 25% of a patient population having PsA. In one embodiment, the effective TNFα inhibitor was previously identified as achieving an ACR50 response in at least about 30% of the patient population. In another embodiment, the effective TNFα inhibitor was previously identified as achieving an ACR50 response in at least about 35% of the patient population. In another embodiment, the effective TNFα inhibitor was previously identified as achieving an ACR50 response in at least about 40% of the patient population. In yet another embodiment, the effective TNFα inhibitor was previously identified as achieving an ACR50 response in at least about 46% of the patient population.

The invention also provides a method of determining the efficacy of a TNFα inhibitor for treating psoriatic arthritis in a subject comprising: determining an ACR70 response of a patient population having psoriatic arthritis and who was administered the TNFα inhibitor, wherein an ACR70 response in at least about 14% of the patient population indicates that the TNFα inhibitor is an effective human TNFα antibody, or antigen-binding portion thereof, for the treatment of psoriatic arthritis in a subject.

In one embodiment, the effective TNFα inhibitor is administered to a subject to treat psoriatic arthritis. In another embodiment, an ACR70 response in at least about 20% of the patient population indicates that the TNFα inhibitor is an effective TNFα inhibitor for the treatment of psoriatic arthritis in a subject. In another embodiment, an ACR70 response in at least about 25% of the patient population indicates that the TNFα inhibitor is an effective TNFα inhibitor for the treatment of psoriatic arthritis in a subject. In yet another embodiment, an ACR70 response in at least about 31% of the patient population indicates that the TNFα inhibitor is an effective TNFα inhibitor for the treatment of psoriatic arthritis in a subject.

The invention further provides a method of treating psoriatic arthritis in a subject comprising administering an effective TNFα inhibitor to the subject such that psoriatic arthritis is treated, wherein the effective TNFα inhibitor was previously identified as achieving an ACR70 response in at least about 20% of a patient population having PsA. In one embodiment, the effective TNFα inhibitor was previously identified as achieving an ACR70 response in at least about 25% of the patient population. In another embodiment, the effective TNFα inhibitor was previously identified as achieving an ACR70 response in at least about 31% of the patient population.

The invention further provides a method of determining the efficacy of a TNFα inhibitor for treating psoriatic arthritis in a subject comprising: determining a PASI50 response of a patient population having psoriatic arthritis who was administered the TNFα inhibitor, wherein a PASI50 response in at least about 73% of the patient population indicates that the TNFα inhibitor is an effective TNFα inhibitor for the treatment of psoriatic arthritis in a subject.

One embodiment comprises administering the effective TNFα inhibitor to a subject to treat psoriatic arthritis. In another embodiment, a PASI50 response in at least about 76% of the patient population indicates that the TNFα inhibitor is an effective TNFα inhibitor for the treatment of psoriatic arthritis in a subject.

The invention also provides a method of treating psoriatic arthritis in a subject comprising administering an effective TNFα inhibitor to the subject such that psoriatic arthritis is treated, wherein the effective TNFα inhibitor was previously identified as achieving a PASI50 response in at least about 70% of the patient population having PsA. In one embodiment, the effective TNFα inhibitor was previously identified as achieving an PASI50 response in at least about 76% of the patient population having psoriatic arthritis.

The invention further provides a method of determining the efficacy of a TNFα inhibitor for treating psoriatic arthritis in a subject comprising: determining a PASI75 response of a patient population having psoriatic arthritis who was administered the TNFα inhibitor, wherein a PASI75 response in at least about 40% of the patient population indicates that the TNFα inhibitor is an effective TNFα inhibitor for the treatment of psoriatic arthritis in a subject. In one embodiment, the invention further comprises administering the effective TNFα inhibitor to a subject to treat psoriatic arthritis. In another embodiment, a PASI75 response in at least about 45% of the patient population indicates that the TNFα inhibitor is an effective TNFα inhibitor for the treatment of psoriatic arthritis in a subject. In another embodiment, a PASI75 response in at least about 50% of the patient population indicates that the TNFα inhibitor is an effective TNFα inhibitor for the treatment of psoriatic arthritis in a subject. In yet another embodiment, a PASI75 response in at least about 55% of the patient population indicates that the TNFα inhibitor is an effective TNFα inhibitor for the treatment of psoriatic arthritis in a subject. In another embodiment, a PASI75 response in at least about 59% of the patient population indicates that the TNFα inhibitor is an effective TNFα inhibitor for the treatment of psoriatic arthritis in a subject.

The invention also provides a method of treating psoriatic arthritis in a subject comprising administering an effective TNFα inhibitor to the subject such that psoriatic arthritis is treated, wherein the effective TNFα inhibitor was previously identified as achieving a PASI75 response in at least about 40% of a patient population having PsA. In one embodiment, the effective TNFα inhibitor was previously identified as achieving an PASI75 response in at least about 59% of the patient population having psoriatic arthritis.

The invention also provides a method of determining the efficacy of a TNFα inhibitor for treating psoriatic arthritis in a subject comprising: determining a PASI90 response of a patient population having psoriatic arthritis and who was administered the TNFα inhibitor, wherein a PASI90 response in at least about 25% of the patient population indicates that the TNFα inhibitor is an effective TNFα inhibitor for the treatment of psoriatic arthritis in a subject. In one embodiment, the invention further comprises administering the effective TNFα inhibitor to a subject to treat psoriatic arthritis. In another embodiment a PASI90 response in at least about 30% of the patient population indicates that the TNFα inhibitor is an effective TNFα inhibitor for the treatment of psoriatic arthritis in a subject. In another embodiment, a PASI90 response in at least about 35% of the patient population indicates that the TNFα inhibitor is an effective TNFα inhibitor for the treatment of psoriatic arthritis in a subject. In yet another embodiment, a PASI90 response in at least about 40% of the patient population indicates that the TNFα inhibitor is an effective TNFα inhibitor for the treatment of psoriatic arthritis in a subject. In another embodiment, a PASI90 response in at least about 42% of the patient population indicates that the TNFα inhibitor is an effective TNFα inhibitor for the treatment of psoriatic arthritis in a subject.

The invention also provides a method of treating psoriatic arthritis in a subject comprising administering an effective TNFα inhibitor to the subject such that psoriatic arthritis is treated, wherein the effective TNFα inhibitor was previously identified as achieving a PASI90 response in at least about 25% of a patient population having PsA. In one embodiment, the effective TNFα inhibitor was previously identified as achieving a PASI90 response in at least about 42% of the patient population.

The invention further provides a method of determining the efficacy of a TNFα inhibitor for treating psoriatic arthritis in a subject comprising: determining a PGA response of “Clear” or “Almost Clear,” of a patient population having psoriatic arthritis and who was administered the TNFα inhibitor, wherein a PGA response of “Clear” or “Almost Clear,” in at least about 40% of the patient population indicates that the TNFα inhibitor is an effective TNFα inhibitor for the treatment of psoriatic arthritis in a subject. One embodiment comprises administering the effective TNFα inhibitor to a subject to treat psoriatic arthritis. In another embodiment, a PGA response of “Clear” or “Almost Clear,” in at least about 45% of the patient population indicates that the TNFα inhibitor is an effective TNFα inhibitor for the treatment of psoriatic arthritis in a subject. In another embodiment, a PGA response of “Clear” or “Almost Clear,” in at least about 50% of the patient population indicates that the TNFα inhibitor is an effective TNFα inhibitor for the treatment of psoriatic arthritis in a subject. In yet another embodiment, a PGA response of “Clear” or “Almost Clear,” in at least about 56% of the patient population indicates that the TNFα inhibitor is an effective TNFα inhibitor for the treatment of psoriatic arthritis in a subject. In another embodiment, a PGA response of “Clear” or “Almost Clear,” in at least about 80% of the patient population indicates that the TNFα inhibitor is an effective TNFα inhibitor for the treatment of psoriatic arthritis in a subject.

The invention also provides a method of treating psoriatic arthritis in a subject comprising administering an effective TNFα inhibitor to the subject such that psoriatic arthritis is treated, wherein the effective TNFα inhibitor was previously identified as achieving a PGA response of “Clear” or “Almost Clear,” in at least about 40% of a patient population having PsA. In one embodiment, the TNFα inhibitor was previously identified as achieving a PGA response of “Clear” or “Almost Clear,” in at least about 45% of the patient population. In another embodiment, the effective TNFα inhibitor was previously identified as achieving a PGA response of “Clear” or “Almost Clear,” in at least about 56% of the patient population.

The invention also provides a method of determining the efficacy of a TNFα inhibitor for treating psoriatic arthritis in a subject comprising: determining a Health Assessment Questionnaire (HAQ) response of a patient population having psoriatic arthritis and who was administered the TNFα inhibitor, wherein an average decrease of about 0.3 in the HAQ score of the patient population indicates that the TNFα inhibitor is an effective TNFα inhibitor for the treatment of psoriatic arthritis in a subject. One embodiment comprises administering the effective TNFα inhibitor to a subject to treat psoriatic arthritis. In another embodiment, an average decrease of about 0.4 in the HAQ score of the patient population indicates that the TNFα inhibitor is an effective TNFα inhibitor for the treatment of psoriatic arthritis in a subject. In another embodiment, an average decrease of about 0.5 in the HAQ score of the patient population indicates that the TNFα inhibitor is an effective TNFα inhibitor for the treatment of psoriatic arthritis in a subject.

The invention also provides a method of treating psoriatic arthritis in a subject comprising administering an effective TNFα inhibitor to the subject such that psoriatic arthritis (PsA) is treated, wherein the effective TNFα inhibitor was previously identified as decreasing the HAQ average score by about 0.3 in a patient population having PsA. In one embodiment, the effective TNFα inhibitor was previously identified as decreasing the HAQ score of the patient population on average by about 0.4. In another embodiment, the effective TNFα inhibitor was previously identified as decreasing the HAQ score of the patient population on average by about 0.5.

The invention further provides a method for treating a human subject having psoriatic arthritis (PsA) who has failed Disease-Modifying Anti-Rheumatic Drug (DMARD) therapy comprising administering to the subject a TNFα inhibitor, such that PsA is treated. In one embodiment, the failed DMARD therapy is failed methotrexate therapy.

The invention further provides a method for treating a human subject having psoriatic arthritis (PsA) who has failed Non-Steroidal Anti-Inflammatory Drug (NSAID) therapy comprising administering to the subject a TNFα inhibitor, such that PsA is treated.

The invention also provides a method for treating a human subject having psoriatic arthritis (PsA) who has failed Disease-Modifying Anti-Rheumatic Drug (DMARD) therapy comprising administering to the subject a monotherapy comprising a TNFα inhibitor, such that PsA is treated. In one embodiment, the monotherapy does not include administration of methotrexate.

The invention further provides a method for inhibiting radiographic progression of joint disease associated with psoriatic arthritis (PsA) in a subject comprising administering a TNFα inhibitor to a subject having PsA, such that radiographic progression of joint disease is inhibited.

The invention also provides a method for decreasing a modified Total Sharp Score (mTSS) of a subject having PsA comprising administering a TNFα inhibitor to a subject having PsA, such that mTSS score of the subject decreases.

The invention further provides a method for inhibiting an increase in a modified Total Sharp Score (mTSS) of a subject having PsA comprising administering a TNFα inhibitor to a subject having PsA, such that mTSS score of the subject does not increase.

In one embodiment, the subject has moderate to severe PsA.

In one embodiment, the TNFα inhibitor is administered to the subject on a biweekly dosing regimen.

In one embodiment, the TNFα inhibitor is administered in combination with an additional agent.

In one embodiment, the TNFα inhibitor is administered subcutaneously.

In one embodiment, the TNFα inhibitor is adalimumab. In another embodiment, 40 mg of adalimumab is administered to the subject.

The invention further provides an article of manufacture comprising a packaging material; a human TNFα antibody, or antigen-binding portion thereof; and a label or package insert contained within the packaging material indicating that human TNFα antibody, or antigen-binding portion thereof, may be used to reduce signs and symptoms of active arthritis in patients having PsA.

The invention also provides an article of manufacture comprising a packaging material; a human TNFα antibody, or antigen-binding portion thereof; and a label or package insert contained within the packaging material indicating that human TNFα antibody, or antigen-binding portion thereof, may be used to inhibit the progression of structural damage in patients having PsA.

The invention further provides an article of manufacture comprising a packaging material; a human TNFα antibody, or antigen-binding portion thereof; and a label or package insert contained within the packaging material indicating that human TNFα antibody, or antigen-binding portion thereof, may be used to improve physical function in patients having PsA. In one embodiment, the human TNFα antibody, or antigen-binding portion thereof, is adalimumab.

The invention provides a method for determining the effectiveness of a TNFα inhibitor for the treatment of moderate to severely active psoriatic arthritis (PsA) in patients having an inadequate response to previous disease-modifying rheumatic drug (DMARD) therapy comprising using a mean baseline HAQ score of a preselected patient population having PsA and a mean HAQ score of the patient population following administration of the TNFα inhibitor, wherein a decrease of about 0.3 in the mean HAQ score following administration of the TNFα inhibitor indicates that the TNFα inhibitor is effective for the treatment of moderate to severely active PsA.

The invention also provides a method for determining the effectiveness of a TNFα inhibitor for the treatment of moderate to severely active psoriatic arthritis (PsA) in patients having an inadequate response to previous disease-modifying rheumatic drug (DMARD) therapy comprising using a mean baseline ACR score of a preselected patient population having PsA and a mean ACR score of the patient population following administration of the TNFα inhibitor, wherein an ACR20 achieved in at least about 57% of the patient population indicates that the TNFα inhibitor is effective for the treatment of moderate to severely active PsA.

The invention further provides a method for determining the effectiveness of a TNFα inhibitor for the treatment of moderate to severely active psoriatic arthritis (PsA) in patients having an inadequate response to previous disease-modifying rheumatic drug (DMARD) therapy comprising using a mean baseline ACR score of a preselected patient population having PsA and a mean ACR score of the patient population following administration of the TNFα inhibitor, wherein an ACR50 achieved in at least about 25% of the patient population indicates that the TNFα inhibitor is effective for the treatment of moderate to severely active PsA.

The invention includes a method for determining the effectiveness of a TNFα inhibitor for the treatment of moderate to severely active psoriatic arthritis (PsA) in patients having an inadequate response to previous disease-modifying rheumatic drug (DMARD) therapy comprising using a mean baseline ACR score of a preselected patient population having PsA and a mean ACR score of the patient population following administration of the TNFα inhibitor, wherein an ACR70 achieved in at least about 14% of the patient population indicates that the TNFα inhibitor is effective for the treatment of moderate to severely active PsA.

The invention also provides a method for determining the effectiveness of a TNFα inhibitor for the treatment of moderate to severely active psoriatic arthritis (PsA) in patients having an inadequate response to previous disease-modifying rheumatic drug (DMARD) therapy comprising using a mean baseline physician's global assessment (PGA) score of a preselected patient population having PsA and a mean PGA score of the patient population following administration of the TNFα inhibitor, wherein a PGA score of “clear” or “almost clear” achieved in at least about 40% of the patient population indicates that the TNFα inhibitor is effective for the treatment of moderate to severely active PsA.

The invention further provides a method for treating moderate to severe PsA in a patient who has failed prior DMARD therapy comprising administering a TNFα inhibitor to the patient every other week, such that the moderate to severe PsA is treated.

The invention includes a method of improving the quality of life of a subject having moderate to severe PsA comprising administering a TNFα inhibitor to the subject such that quality of life is improved, wherein the improvement in quality of life is determined by at least one improvement selected from the group consisting of an decrease in the HAQ score of at least about 0.3 from a predetermined baseline HAQ score, an increase in the FACIT-Fatigue of at least about 6.5 from a predetermined baseline FACIT score, an increase of at least about 9.3 in the physical component summary (PCS) of the SF-36 score from a predetermined baseline PCS score, an increase of at least about 1.6 in the mental component summary (MCS) of the SF-36 score from a predetermined baseline MCS score, and a decrease of at least about 5.6 in the DLQI score from a predetermined baseline DLQI score.

In one embodiment of the invention, the patient population has ≧3 swollen joins and ≧3 tender joints.

In one embodiment, the invention provides a method for predicting an improvement in the quality of life of a subject having PsA using an indicator selected from the group consisting of HAQ-DI score, PASI score, and a TJC score. In another embodiment, the invention provides a means for determining the efficacy of a TNFα inhibitor for improving the quality of life in a subject(s) (or preselected patient population) having PsA, wherein an improvement in an index selected from the group consisting of HAQ-DI score, PASI score, and a TJC score indicates that the TNFα inhibitor is effective for improving the quality of life of a subject having PsA. In one embodiment, the invention further comprises administering to a subject having PsA a TNFα inhibitor identified as being effective for improving the quality of life in a patient population having PsA.

The invention further provides a method for monitoring the effectiveness of a TNFα inhibitor for the treatment of psoriatic arthritis (PsA) in a human subject comprising using a mean baseline Disease Activity Score (DAS)28 score of a patient population having PsA and a DAS28 score of the patient population following administration of the TNFα inhibitor, wherein a mean decrease in the DAS28 score of at least about −1.7 indicates that the TNFα inhibitor is effective at treating PsA. In one embodiment, the TNFα inhibitor has already been administered to the pre-selected patient population.

In another embodiment, the TNFα antibody, or antigen-binding portion thereof, is administered subcutaneously.

In still another embodiment, the TNFα antibody, or antigen-binding portion thereof, is infliximab or golimumab. In one embodiment, the patient or patient population is administered methotrexate in combination with the TNFα inhibitor. The invention further provides an article of manufacture comprising a packaging material; a TNFα antibody; and a label or package insert contained within the packaging material indicating that in studies of the TNFα inhibitor for the treatment of PsA, common adverse events (AEs) include at least one disorder selected from the group consisting of injection site pain, upper respiratory tract infection, Ps aggravation, diarrhea, back pain, PsA aggravated, and headache.

In one embodiment of the invention, the TNFα inhibitor is selected from the group consisting of a TNFα antibody, or an antigen-binding portion thereof, a TNF fusion protein, or a recombinant TNF binding protein. In another embodiment, the TNF fusion protein is etanercept.

In still another embodiment, the TNFα antibody, or antigen-binding portion thereof, is selected from the group consisting of a chimeric antibody, a humanized antibody, and a multivalent antibody. In one embodiment, the TNFα antibody, or antigen-binding portion thereof, is a human antibody.

In another embodiment, the TNFα antibody, or antigen-binding portion thereof, is an isolated human antibody that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

In still another embodiment, the TNFα antibody is an isolated human antibody, or antigen-binding portion thereof, with the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

In one embodiment of the invention, the TNFα antibody is an isolated human antibody, or an antigen binding portion thereof, with a light chain variable region (LCVR) comprising the amino acid sequence of SEQ ID NO: 1 and a heavy chain variable region (HCVR) comprising the amino acid sequence of SEQ ID NO: 2.

In one embodiment, human TNFα antibody, or antigen-binding portion thereof, is adalimumab. In one embodiment, the TNFα antibody, or antigen-binding portion thereof, is a 40 mg dose.

### BRIEF DESCRIPTION OF THE DRAWINGS

FIG. 1 shows the design of Study X, used to evaluate the efficacy of adalimumab compared with placebo in patients with moderately to severely active psoriatic arthritis (PsA) who had an inadequate response to DMARD therapy. Subjects completing Week 12 were eligible to continue in the open-label extension study.

FIG. 2 graphically depicts ACR20/50/70 response by week.

FIG. 3 shows the mean percent reduction in target lesion score by week.

FIG. 4 graphically depicts the percent improvement in health utility at 24 weeks by treatment group and psoriasis Body Surface Area (BSA) percent.

FIG. 5 shows percent improvement in health utility at 24 weeks by treatment group, psoriasis BSA percent and response type (R=Responders; NR=Non-Responders).

FIG. 6 shows ACR response (ACR20, ACR50, ACR70) by week.

FIG. 7 depicts the PASI response by week.

FIG. 8 depicts patient characteristics and response results used to model efficacy.

FIG. 9 graphically depicts joint-related direct costs (surgical procedures and hospitalizations) as a function of HAQ. Analysis assumes the relationship is equal for both PsA and rheumatoid arthritis (see also Michaud K. et al. Arthritis Rheum. 2003; 48:2750-62).

FIG. 10 graphically depicts psoriasis-related direct costs (corticosteroids, retinoids, UV-B) as a function of PASI. This analysis assumes costs are for prescriptions or visits to clinics. The unit costs (AWP) are as follows: acitretin 25 mg, $14.91; folic acid 1 mg, $0.0085; retamethasone valerate 15 mg, $5.20; hydrocortisone 30 g, $1.12; clobetasol 30 g, $16.50; topical coal tar, $14.69; dovonex 100 g, $162.13; and broadband UV-B per session, $41.44.

FIG. 11 outlines the study design of Study G. Patients completing Week 24 of the study were eligible to continue in an open-label extension study.

FIG. 12 graphically depicts the mean percentage change in DAS28 scores over time in patients treated with adalimumab versus placebo. p<0.001 for adalimumab vs. placebo at all time points after baseline (Last observation carried forward).

FIG. 13 graphically depicts the ACR20/50/70 response of patients treated with adalimumab and placebo through Week 24. †p<0.01; *p<0.001 adalimumab vs. placebo (non-responder imputation).

FIG. 14 depicts the mean change in TJC and SJC in patients treated with adalimumab and placebo through Week 24. *p<0.001; †p<0.01; ‡p<0.05, adalimumab vs. placebo (Last observation carried forward).

FIG. 15 outlines the study design of Study G, including the open-label extension period. Weekly adalimumab dosing was allowed on or after Week 36 in patients with ≦20% improvement in TJC and SJC.

FIG. 16 graphically depicts ACR 20/50/70 responses over time, to 48 weeks. *p<0.001; †p<0.01; ‡p<0.05, adalimumab vs. placebo (non-responder imputation).

FIG. 17 depicts PASI responses over 48 weeks. *p<0.001, adalimumab vs. placebo. PASI50/75/90 is by non-responder imputation. Mean percentage improvement in PASI is by last observation carried forward. Patients in the placebo group at Week 48 started adalimumab treatment at Week 24.

### DETAILED DESCRIPTION OF THE INVENTION

### I. Definitions

The term “human TNFα” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNFα is intended to include recombinant human TNFα (rhTNFα), which can be prepared by standard recombinant expression methods or purchased commercially (R & D Systems, Catalog No. 210-TA, Minneapolis, Minn.). TNFα is also referred to as TNF.

The term “TNFα inhibitor” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (Enbrel®, Amgen; described in WO 91/03553 and WO 09/406476, incorporated by reference herein). In another embodiment, the TNFα inhibitor is a recombinant TNF binding protein (r-TBP-I) (Serono).

The term “antibody,” as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The antibodies of the invention are described in further detail in U.S. Pat. Nos. 6,090,382; 6,258,562; and 6,509,015, each of which is incorporated herein by reference in its entirety.

The term “antigen-binding portion” or “antigen-binding fragment” of an antibody (or simply “antibody portion”), as used herein, refers to one or more fragments of an antibody that retain the ability to specifically bind to an antigen (e.g., hTNFα). It has been shown that the antigen-binding function of an antibody can be performed by fragments of a full-length antibody. Binding fragments include Fab, Fab′, F(ab′)₂, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH or VL domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). The antibody portions of the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

A “conservative amino acid substitution,” as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In a preferred embodiment of the invention, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immmunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Such chimeric, humanized, human, and dual specific antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539.

An “isolated antibody,” as used herein, is intended to refer to an antibody that is substantially free of other antibodies having different antigenic specificities (e.g., an isolated antibody that specifically binds hTNFα is substantially free of antibodies that specifically_bind antigens other than hTNFα). An isolated antibody that specifically binds hTNFα may, however, have cross-reactivity to other antigens, such as TNFα molecules from other species. Moreover, an isolated antibody may be substantially free of other cellular material and/or chemicals.

A “neutralizing antibody,” as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance,” as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jönsson et al. (1993) Ann. Biol. Clin. 51:19; Jönsson et al. (1991) Biotechniques 11:620-627; Johnsson et al. (1995) J. Mol. Recognit. 8:125; and Johnnson et al. (1991) Anal. Biochem. 198:268.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of the inhibitor required to inhibit the biological endpoint of interest, e.g., neutralize cytotoxicity activity.

The term “dose,” as used herein, refers to an amount of TNFα inhibitor, e.g., TNFα antibody, which is administered to a subject.

The term “dosing”, as used herein, refers to the administration of a substance (e.g., an anti-TNFα antibody) to achieve a therapeutic objective (e.g., treatment of psoriatic arthritis).

A “dosing regimen” describes a treatment schedule for a TNFα inhibitor, e.g., a treatment schedule over a prolonged period of time and/or throughout the course of treatment, e.g. administering a first dose of a TNFα inhibitor at week 0 followed by a second dose of a TNFα inhibitor on a biweekly dosing regimen.

The terms “biweekly dosing regimen”, “biweekly dosing”, and “biweekly administration”, as used herein, refer to the time course of administering a substance (e.g., an anti-TNFα antibody) to a subject to achieve a therapeutic objective, e.g, throughout the course of treatment. The biweekly dosing regimen is not intended to include a weekly dosing regimen. Preferably, the substance is administered every 9-19 days, more preferably, every 11-17 days, even more preferably, every 13-15 days, and most preferably, every 14 days. In one embodiment, the biweekly dosing regimen is initiated in a subject at week 0 of treatment. In one embodiment, biweekly dosing includes a dosing regimen wherein doses of a TNFα inhibitor are administered to a subject every other week beginning at week 0. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “combination therapy”, as used herein, refers to the administration of two or more therapeutic substances, e.g., an anti-TNFα antibody and another drug. The other drug(s) may be administered concomitant with, prior to, or following the administration of an anti-TNFα antibody.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “treatment,” as used within the context of the present invention, is meant to include therapeutic treatment, as well as prophylactic or suppressive measures, for the treatment of psoriatic arthritis. For example, in one embodiment, the term “treatment” or “treating” refers to reducing signs and symptoms of active arthritis. In one embodiment, the term “treatment” or “treating” refers to inhibiting the progression of structural damage in patients with psoriatic arthritis. In one embodiment, the term “treatment” or “treating” refers to improving physical function in patients with psoriatic arthritis. The term treatment may, for example, include administration of a TNFα inhibitor prior to or following the onset of psoriatic arthritis thereby preventing or removing signs of the disease or disorder. As another example, administration of a TNFα inhibitor after clinical manifestation of psoriatic arthritis to combat the symptoms and/or complications and disorders associated with psoriatic arthritis comprises “treatment” of the disease. Further, administration of the agent after onset and after clinical symptoms and/or complications have developed where administration affects clinical parameters of the disease or disorder and perhaps amelioration of the disease, comprises “treatment” of psoriatic arthritis.

Those “in need of treatment” include mammals, such as humans, already having psoriatic arthritis, including those in which the disease or disorder is to be prevented.

Various aspects of the invention are described in further detail herein.

The invention provides improved uses and compositions for treating psoriatic arthritis with a TNFα inhibitor, e.g., a human TNFα antibody, or an antigen-binding portion thereof. Compositions and articles of manufacture, including kits, relating to the methods and uses for treating psoriatic arthritis are also contemplated as part of the invention.

### II. TNF Inhibitors

A TNFα inhibitor which is used in the methods and compositions of the invention includes any agent which interferes with TNFα activity. In a preferred embodiment, the TNFα inhibitor can neutralize TNFα activity, particularly detrimental TNFα activity which is associated with psoriatic arthritis, and related complications and symptoms.

In one embodiment, the TNFα inhibitor used in the invention is an TNFα antibody (also referred to herein as a TNFα antibody), or an antigen-binding fragment thereof, including chimeric, humanized, and human antibodies. Examples of TNFα antibodies which may be used in the invention include, but not limited to, infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein.

Other examples of TNFα inhibitors which may be used in the methods and compositions of the invention include etanercept (Enbrel, described in WO 91/03553 and WO 09/406476), soluble TNF receptor Type I, a pegylated soluble TNF receptor Type I (PEGs TNF-R1), p55TNFR1gG (Lenercept), and recombinant TNF binding protein (r-TBP-I) (Serono).

In one embodiment, the term “TNFα inhibitor” excludes infliximab. In one embodiment, the term “TNFα inhibitor” excludes adalimumab. In another embodiment, the term “TNFα inhibitor” excludes adalimumab and infliximab.

In one embodiment, the term “TNFα inhibitor” excludes etanercept, and, optionally, adalimumab, infliximab, or adalimumab and infliximab.

In one embodiment, the term “TNFα antibody” excludes infliximab. In one embodiment, the term “TNFα antibody” excludes adalimumab. In another embodiment, the term “TNFα antibody” excludes adalimumab and infliximab.

In one embodiment, the invention features uses and composition for treating or determining the efficacy of a TNFα inhibitor for the treatment of Psoriatic arthritis, wherein the TNFα antibody is an isolated human antibody, or antigen-binding portion thereof, that binds to human TNFα with high affinity and a low off rate, and also has a high neutralizing capacity. Preferably, the human antibodies used in the invention are recombinant, neutralizing human anti-hTNFα antibodies. The most preferred recombinant, neutralizing antibody of the invention is referred to herein as D2E7, also referred to as HUMIRA® or adalimumab (the amino acid sequence of the D2E7 VL region is shown in SEQ ID NO: 1; the amino acid sequence of the D2E7 VH region is shown in SEQ ID NO: 2). The properties of D2E7 (adalimumab/HUMIRA®) have been described in Salfeld et al., U.S. Pat. Nos. 6,090,382, 6,258,562, and 6,509,015, which are each incorporated by reference herein.

In one embodiment, the method of the invention includes determining the efficacy of a human TNFα antibody, e.g., D2E7 antibodies and antibody portions, D2E7-related antibodies and antibody portions, or other human antibodies and antibody portions with equivalent properties to D2E7, such as high affinity binding to hTNFα with low dissociation kinetics and high neutralizing capacity, for the treatment of psoriatic arthritis. In one embodiment, the invention provides treatment with an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the isolated human antibody, or antigen-binding portion thereof, dissociates from human TNFα with a K_{off} of 5×10⁻⁴ s⁻¹ or less, or even more preferably, with a K_{off} of 1×10⁻⁴ s⁻¹ or less. More preferably, the isolated human antibody, or antigen-binding portion thereof, neutralizes human TNFα cytotoxicity in a standard in vitro L929 assay with an IC₅₀ of 1×10⁻⁸ M or less, even more preferably with an IC₅₀ of 1×10⁻⁹ M or less and still more preferably with an IC₅₀ of 1×10⁻¹⁰ M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to treating Psoriatic arthritis by administering human antibodies that have slow dissociation kinetics for association with hTNFα and that have light and heavy chain CDR3 domains that structurally are identical to or related to those of D2E7. Position 9 of the D2E7 VL CDR3 can be occupied by Ala or Thr without substantially affecting the K_{off}. Accordingly, a consensus motif for the D2E7 VL CDR3 comprises the amino acid sequence: Q-R-Y-N-R-A-P-Y-(T/A) (SEQ ID NO: 3). Additionally, position 12 of the D2E7 VH CDR3 can be occupied by Tyr or Asn, without substantially affecting the K_{off}. Accordingly, a consensus motif for the D2E7 VH CDR3 comprises the amino acid sequence: V-S-Y-L-S-T-A-S-S-L-D-(Y/N) (SEQ ID NO: 4). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the K_{off}. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNFα. Positions 2 and 5 of the D2E7 VL CDR3 and positions 1 and 7 of the D2E7 VH CDR3 appear to be critical for interaction with hTNFα and thus, conservative amino acid substitutions preferably are not made at these positions (although an alanine substitution at position 5 of the D2E7 VL CDR3 is acceptable, as described above) (see U.S. Pat. No. 6,090,382).

Accordingly, in another embodiment, the antibody or antigen-binding portion thereof preferably contains the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNFα with a K_{off} of 5×10⁻⁴ s⁻¹ or less. Even more preferably, the antibody, or antigen-binding portion thereof, dissociates from human TNFα with a K_{off} of 1×10⁻⁴ s⁻¹ or less.

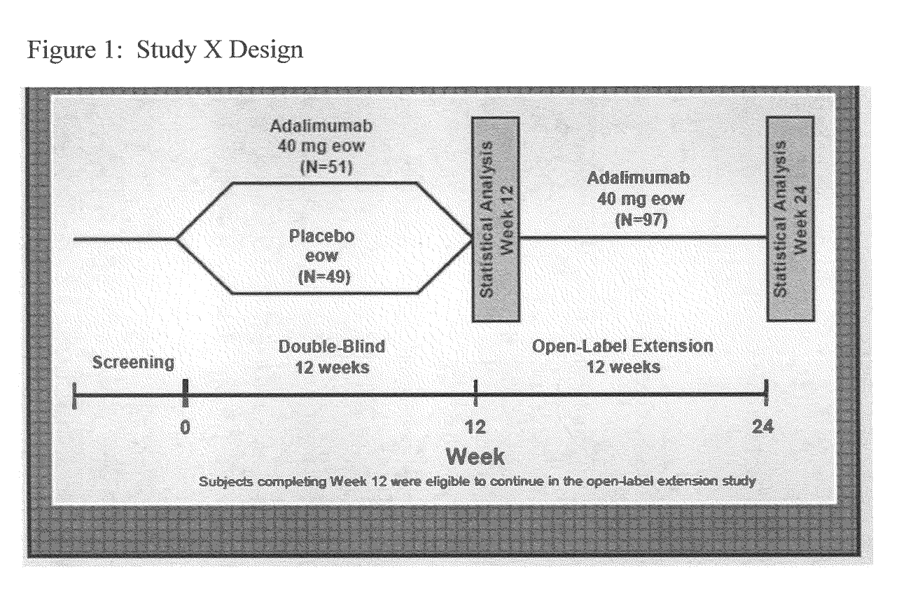

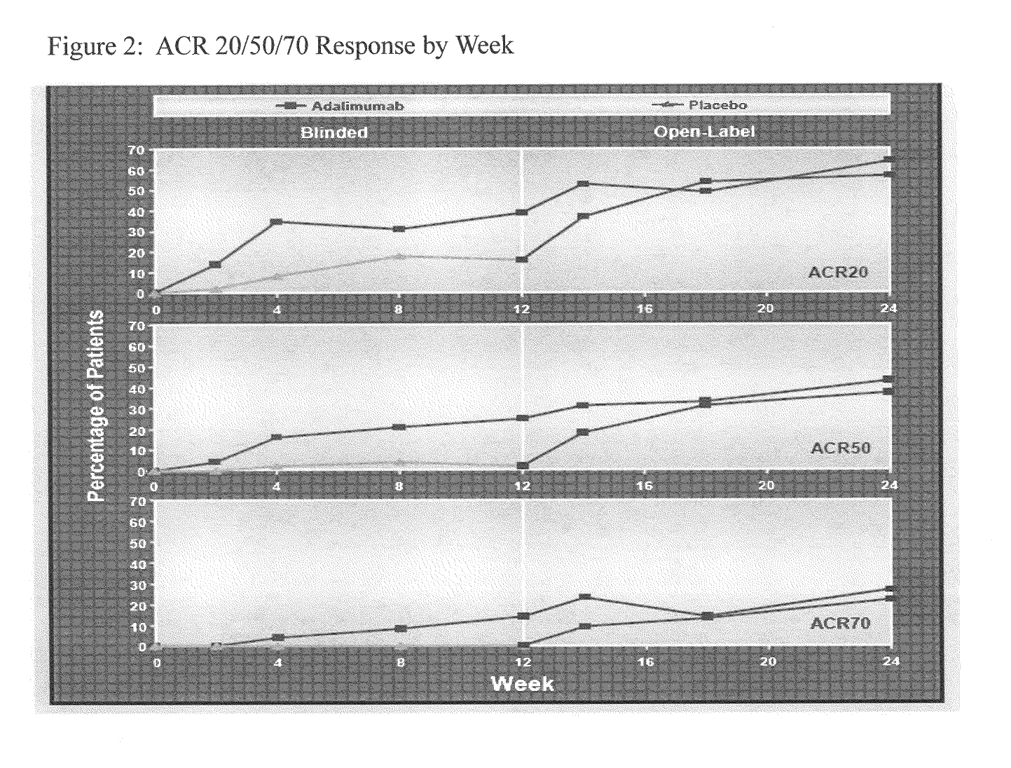

In yet another embodiment, the antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. Preferably, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). Even more preferably, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL preferably are from the V_{K}I human germline family, more preferably from the A20 human germline Vk gene and most preferably from the D2E7 VL framework sequences shown in FIGS. 1A and 1B of U.S. Pat. No. 6,090,382. The framework regions for VH preferably are from the V_{H}3 human germline family, more preferably from the DP-31 human germline VH gene and most preferably from the D2E7 VH framework sequences shown in FIGS. 2A and 2B of U.S. Pat. No. 6,090,382.

Accordingly, in another embodiment, the antibody or antigen-binding portion thereof preferably contains a light chain variable region (LCVR) comprising the amino acid sequence of SEQ ID NO: 1 (i.e., the D2E7 VL) and a heavy chain variable region (HCVR) comprising the amino acid sequence of SEQ ID NO: 2 (i.e., the D2E7 VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention includes uses of an isolated human antibody, or an antigen-binding portions thereof, containing D2E7-related VL and VH CDR3 domains. For example, antibodies, or antigen-binding portions thereof, with a light chain variable region (LCVR) having a CDR3 domain comprising an amino acid sequence selected from the group consisting of SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

The TNFα antibody used in the methods and compositions of the invention may be modified for improved treatment of Psoriatic arthritis. In some embodiments, the TNFα antibody or antigen binding fragments thereof, is chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat Psoriatic arthritis by administration of the TNFα antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, TNFα antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion used in the methods of the invention can be derivatized or linked to another functional molecule (e.g., another peptide or protein). Accordingly, the antibodies and antibody portions of the invention are intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

An antibody, or antibody portion, used in the methods and compositions of the invention, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express adalimumab (D2E7) or an adalimumab (D2E7)-related antibody, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V₇₈ Segments Reveals a Strong Bias in their Usage” Eur. J. Immunol. 24:827-836; the contents of each of which are expressly incorporated herein by reference). To obtain a DNA fragment encoding the heavy chain variable region of D2E7, or a D2E7-related antibody, a member of the V_{H}3 family of human germline VH genes is amplified by standard PCR. Most preferably, the DP-31 VH germline sequence is amplified. To obtain a DNA fragment encoding the light chain variable region of D2E7, or a D2E7-related antibody, a member of the V_{K}I family of human germline VL genes is amplified by standard PCR. Most preferably, the A20 VL germline sequence is amplified. PCR primers suitable for use in amplifying the DP-31 germline VH and A20 germline VL sequences can be designed based on the nucleotide sequences disclosed in the references cited supra, using standard methods.

Once the germline VH and VL fragments are obtained, these sequences can be mutated to encode the D2E7 or D2E7-related amino acid sequences disclosed herein. The amino acid sequences encoded by the germline VH and VL DNA sequences are first compared to the D2E7 or D2E7-related VH and VL amino acid sequences to identify amino acid residues in the D2E7 or D2E7-related sequence that differ from germline. Then, the appropriate nucleotides of the germline DNA sequences are mutated such that the mutated germline sequence encodes the D2E7 or D2E7-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Moreover, it should be noted that if the “germline” sequences obtained by PCR amplification encode amino acid differences in the framework regions from the true germline configuration (i.e., differences in the amplified sequence as compared to the true germline sequence, for example as a result of somatic mutation), it may be desirable to change these amino acid differences back to the true germline sequences (i.e., “backmutation” of framework residues to the germline configuration).

Once DNA fragments encoding D2E7 or D2E7-related VH and VL segments are obtained (by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions used in the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the D2E7 or D2E7-related light or heavy chain sequences, the expression vector may already carry antibody constant region sequences. For example, one approach to converting the D2E7 or D2E7-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors used in the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNFα. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNFα by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of the foregoing, nucleic acid, vector and host cell compositions that can be used for recombinant expression of the antibodies and antibody portions used in the invention include nucleic acids, and vectors comprising said nucleic acids, comprising the human TNFα antibody adalimumab (D2E7). The nucleotide sequence encoding the D2E7 light chain variable region is shown in SEQ ID NO: 36. The CDR1 domain of the LCVR encompasses nucleotides 70-102, the CDR2 domain encompasses nucleotides 148-168 and the CDR3 domain encompasses nucleotides 265-291. The nucleotide sequence encoding the D2E7 heavy chain variable region is shown in SEQ ID NO: 37. The CDR1 domain of the HCVR encompasses nucleotides 91-105, the CDR2 domain encompasses nucleotides 148-198 and the CDR3 domain encompasses nucleotides 295-330. It will be appreciated by the skilled artisan that nucleotide sequences encoding D2E7-related antibodies, or portions thereof (e.g., a CDR domain, such as a CDR3 domain), can be derived from the nucleotide sequences encoding the D2E7 LCVR and HCVR using the genetic code and standard molecular biology techniques.

Recombinant human antibodies of the invention in addition to D2E7 or an antigen binding portion thereof, or D2E7-related antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating and screening antibody display libraries can be found in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-65; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982.

In a preferred embodiment, to isolate human antibodies with high affinity and a low off rate constant for hTNFα, a murine anti-hTNFα antibody having high affinity and a low off rate constant for hTNFα (e.g., MAK 195, the hybridoma for which has deposit number ECACC 87 050801) is first used to select human heavy and light chain sequences having similar binding activity toward hTNFα, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J 12:725-734. The scFv antibody libraries preferably are screened using recombinant human TNFα as the antigen.

Once initial human VL and VH segments are selected, “mix and match” experiments, in which different pairs of the initially selected VL and VH segments are screened for hTNFα binding, are performed to select preferred VL/VH pair combinations. Additionally, to further improve the affinity and/or lower the off rate constant for hTNFα binding, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to hTNFα and sequences that exhibit high affinity and a low off rate for hTNFα binding can be selected.

Following screening and isolation of an anti-hTNFα antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in above.

Methods of isolating human neutralizing antibodies with high affinity and a low off rate constant for hTNFα are described in U.S. Pat. Nos. 6,090,382, 6,258,562, and 6,509,015, each of which is incorporated by reference herein.

Antibodies, antibody-portions, and other TNFα inhibitors for use in the methods of the invention, can be incorporated into pharmaceutical compositions suitable for administration to a subject. Typically, the pharmaceutical composition comprises an antibody, antibody portion, or other TNFα inhibitor, and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody, antibody portion, or other TNFα inhibitor.

The compositions for use in the methods and compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies or other TNFα inhibitors. The preferred mode of administration is parenteral (e.g., intravenous, subcutaneous, intraperitoneal, intramuscular). In a preferred embodiment, the antibody or other TNFα inhibitor is administered by intravenous infusion or injection. In another preferred embodiment, the antibody or other TNFα inhibitor is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, antibody portion, or other TNFα inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In one embodiment, the invention includes pharmaceutical compositions comprising an effective TNFα inhibitor and a pharmaceutically acceptable carrier, wherein the effective TNFα inhibitor may be used to treat psoriatic arthritis.

In one embodiment, the antibody or antibody portion for use in the methods of the invention is incorporated into a pharmaceutical formulation as described in PCT/IB03/04502 and U.S. Appin. No. 20040033228, incorporated by reference herein. This formulation includes a concentration 50 mg/ml of the antibody D2E7 (adalimumab), wherein one pre-filled syringe contains 40 mg of antibody for subcutaneous injection.

The antibodies, antibody-portions, and other TNFα inhibitors of the present invention can be administered by a variety of methods known in the art, although for many therapeutic applications, the preferred route/mode of administration is parenteral, e.g., subcutaneous injection. In another embodiment, administration is via intravenous injection or infusion.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, Robinson, ed., Dekker, Inc., New York, 1978.

In one embodiment, the TNFα antibodies and inhibitors used in the invention are delivered to a subject subcutaneously. In one embodiment, the subject administers the TNFα inhibitor, including, but not limited to, TNFα antibody, or antigen-binding portion thereof, to himself/herself.

The TNFα antibodies and inhibitors used in the invention may also be administered in the form of protein crystal formulations which include a combination of protein crystals encapsulated within a polymeric carrier to form coated particles. The coated particles of the protein crystal formulation may have a spherical morphology and be microspheres of up to 500 micro meters in diameter or they may have some other morphology and be microparticulates. The enhanced concentration of protein crystals allows the antibody of the invention to be delivered subcutaneously. In one embodiment, the TNFα antibodies of the invention are delivered via a protein delivery system, wherein one or more of a protein crystal formulation or composition, is administered to a subject with a TNFα-related disorder. Compositions and methods of preparing stabilized formulations of whole antibody crystals or antibody fragment crystals are also described in WO 02/072636, which is incorporated by reference herein. In one embodiment, a formulation comprising the crystallized antibody fragments described in PCT/IB03/04502 and U.S. Appin. No. 20040033228, incorporated by reference herein, are used to treat rheumatoid arthritis using the treatment methods of the invention.

In certain embodiments, an antibody, antibody portion, or other TNFα inhibitor of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion for use in the methods of the invention is coformulated with and/or coadministered with one or more additional therapeutic agents, including an Psoriatic arthritis inhibitor or antagonist. For example, an anti-hTNFα antibody or antibody portion of the invention may be coformulated and/or coadministered with one or more additional antibodies that bind other targets associated with TNFα related disorders (e.g., antibodies that bind other cytokines or that bind cell surface molecules), one or more cytokines, soluble TNFα receptor (see e.g., PCT Publication No. WO 94/06476) and/or one or more chemical agents that inhibit hTNFα production or activity (such as cyclohexane-ylidene derivatives as described in PCT Publication No. WO 93/19751) or any combination thereof. Furthermore, one or more antibodies of the invention may be used in combination with two or more of the foregoing therapeutic agents. Such combination therapies may advantageously utilize lower dosages of the administered therapeutic agents, thus avoiding possible side effects, complications or low level of response by the patient associated with the various monotherapies.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFα inhibitor may vary according to factors such as the disease state, age, sex, and weight of the individual, and the ability of the antibody, antibody portion, other TNFα inhibitor to elicit a desired response in the individual. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Additional description regarding methods and uses of the invention comprising administration of a TNFα inhibitor are described in Part III and the Examples section of this specification.

The invention also pertains to packaged pharmaceutical compositions or kits for administering the anti-TNF antibodies of the invention for the treatment of psoriatic arthritis. In one embodiment of the invention, the kit comprises a TNFα inhibitor, such as an antibody and instructions for administration of the TNFα inhibitor for treatment of psoriatic arthritis. The instructions may describe how, e.g., subcutaneously, and when, e.g., at week 0, week 2, week 4, etc., the different doses of TNFα inhibitor shall be administered to a subject for treatment.

Another aspect of the invention pertains to kits containing a pharmaceutical composition comprising a TNFα inhibitor, such as an antibody, and a pharmaceutically acceptable carrier and one or more pharmaceutical compositions each comprising an additional therapeutic agent useful for treating Psoriatic arthritis, and a pharmaceutically acceptable carrier. Alternatively, the kit comprises a single pharmaceutical composition comprising an anti-TNFα antibody, one or more drugs useful for treating psoriatic arthritis, and a pharmaceutically acceptable carrier. The instructions may describe how, e.g., subcutaneously, and when, e.g., at week 0, week 2, week 4, etc., the different doses of TNFα inhibitor and/or the additional therapeutic agent shall be administered to a subject for treatment.

The kit may contain instructions for dosing of the pharmaceutical compositions for the treatment of psoriatic arthritis. Additional description regarding articles of manufacture of the invention are described in subsection III.

The package or kit alternatively can contain the TNFα inhibitor and it can be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### III. Uses and Compositions for Treating Psoriatic Arthritis

Tumor necrosis factor has been implicated in the pathophysiology of psoriatic arthritis (Partsch et al. (1998) Ann Rheum Dis. 57:691; Ritchlin et al. (1998) J Rheumatol. 25:1544). As referred to herein, psoriatic arthritis (PsA) refers to chronic inflammatory arthritis which is associated with psoriasis. Psoriasis is a common chronic skin condition that causes red patches on the body. About 1 in 20 individuals with psoriasis will develop arthritis along with the skin condition, and in about 75% of cases, psoriasis precedes the arthritis. PsA exhibits itself in a variety of ways, ranging from mild to severe arthritis, wherein the arthritis usually affects the fingers and the spine. When the spine is affected, the symptoms may be similar to those of ankylosing spondylitis. The invention provides improved methods for treating PsA with a TNFα antibody, or antigen-binding fragment thereof.

Treatment of psoriatic arthritis may be determined according to standard clinical definitions. For example, primary efficacy for signs and symptoms can be measured via American College of Rheumatology preliminary criteria for improvement (ACR). ACR criteria measures improvement in tender or swollen joint counts and improvement in three of the following five parameters: acute phase reactant (such as sedimentation rate); patient assessment; physician assessment; pain scale; and disability/functional questionnaire. ACR criteria is indicated as ACR 20 (a 20 percent improvement in tender or swollen joint counts as well as 20 percent improvement in three of the other five criteria), ACR 50 (a 50 percent improvement in tender or swollen joint counts as well as 50 percent improvement in three of the other five criteria), and ACR 70 (a 70 percent improvement in tender or swollen joint counts as well as 70 percent improvement in three of the other five criteria).

Improvements in the skin component of PsA in a subject can be monitored by the subject's Psoriasis Area and Severity Index Score (PASI). The method for determining the PASI has been described in Fredriksson and Pettersson (1978) Dermatologica 157:238 and Marks et al. (1989) Arch Dermatol 125:235. Briefly, the index is based on evaluation of four anatomic sites, including the head, upper extremities, trunk, and lower extremities, for erythema, induration, and desquamation using a 5 point scale (0=no symptoms; 1=slight; 2=moderate; 3=marked; 4=very marked). Based on the extent of lesions in a given anatomic site, the area affected is assigned a numerical value (0=0; 1=<10%; 2=10-29%; 3=30-49%; 4=50-69%; 5=70=89%; 6=90-100%). The PASI score is then calculated, wherein the possible range of PASI score is 0.0 to 72.0 with the highest score representing complete erythroderma of the severest degree.

The invention also provides methods for improving scores indicative of treatment of PsA by administering a TNF_{α} inhibitor, e.g., aTNF_{α} antibody, or antigen-binding fragment thereof, including HAQ (including HAQ-DI), ACR, TJC, PGA, FACIT-F, DLQI, and Sf-36. Multiple other evaluations which may be performed during treatment include Psoriatic Arthritis Response Criteria (PsARC), quality of life measurements, and skin evaluations to determine efficacy on psoriasis lesions (psorasis area severity index (PASI) and target lesion evaluations).

In one embodiment, the invention provides a method for treating psoriatic arthritis in a subject comprising administering a TNFα inhibitor, e.g., a human TNFα antibody, or an antigen-binding portion thereof, to the subject, such that the psoriatic arthritis is treated. In one embodiment, the invention describes a use of a human TNFα antibody, or antigen-binding portion thereof, in the manufacture of a medicament for treating psoriatic arthritis in a subject. In one embodiment, efficacy of treatment of psoriatic arthritis is determined by achievement of an ACR20, ACR50 or ACR70 response, or a PASI50, PASI75, or PASI90 response in the subject.

In another embodiment, efficacy of treatment of psoriatic arthritis is determined by measuring whether the a TNFα inhibitor, e.g., a human TNFα antibody, or an antigen-binding portion thereof, can inhibit or decrease radiographic progression, e.g., radiographic progression of joint disease associated with PsA. Radiographic progression may be determined using a radiographic scoring method, such as the Modified Total Sharp Score (mTSS) was determined according to the following criteria: joint space narrowing was assessed at 48 sites, each site receiving a score between 0-4, and erosion was assessed at 54 sites, each site receiving a score between 0-7. The range of possible scores for joint space narrowing was consequently 0-192, and the range of possible scores for erosion was 0-378. The sum of these values determined the mTSS, which could range from 0-570. Other radiographic findings associated with PsA include phalangeal tuft resorption (measurable at 12 sites), subluxation (26 sites), pencil-in-cup (18 sites), periostitis (38 sites), and juxta-articular periostitis (52 sites). Other methods for determining radiographic progression of disease, such as PsA, are described in Boini et al. Ann Rheum Dis. 2001 September; 60(9): 817-827.

Thus, the invention provides a method for inhibiting radiographic progression of joint disease associated with psoriatic arthritis (PsA) in a subject comprising administering a TNFα inhibitor to a subject having PsA, such that radiographic progression of joint disease is inhibited. The invention also provides a method for decreasing a modified Total Sharp Score (mTSS) of a subject having PsA comprising comprising administering a TNFα inhibitor to a subject having PsA, such that mTSS score of the subject decreases, or, alternatively, the mTSS score of the subject does not increase.

Methods of treatment described herein may include administration of a TNFα inhibitor to a subject to achieve a therapeutic goal, e.g., achievement of an ACR20, ACR50, or ACR70 response, or a PASI50, PASI75, or PASI90 response. Also included in the scope of the invention are uses of a TNFα inhibitor in the manufacture of a medicament to achieve a therapeutic goal, e.g., achievement of an ACR20, ACR50, or ACR70 response, or a PASI50, PASI75, or PASI90 response. Thus, where methods are described herein, it is also intended to be part of this invention that the use of the TNFα inhibitor in the manufacture of a medicament for the purpose of the method is also considered within the scope of the invention. Likewise, where a use of a TNFα inhibitor in the manufacture of a medicament for the purpose of achieving a therapeutic goal is described, methods of treatment resulting in the therapeutic goal are also intended to be part of the invention.

Other methods for evaluating the treatment of PsA are described below in section IV and the examples section.

The invention also provides a method for treating PsA comprising administering a TNFα inhibitor, such as a TNFα antibody, or an antigen-binding portion thereof, as a monotherapy, i.e., not in combination with an additional agent.

In one embodiment, the TNFα antibody, or an antigen-binding portion thereof, may be administered to the subject on a biweekly dosing regimen in order to achieve the methods of the invention. In one embodiment, biweekly dosing includes a dosing regimen wherein doses of a TNFα inhibitor are administered to a subject every other week beginning at week 0. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc.

In one embodiment, treatment of psoriatic arthritis is achieved by administering a human TNFα antibody, or an antigen-binding portion thereof, to a subject having psoriatic arthritis, wherein the human TNFα antibody, or an antigen-binding portion thereof, is administered on a biweekly dosing regimen. In one embodiment, the human TNFα antibody, or an antigen-binding portion thereof, is administered in a dose of about 40 mg. In one embodiment, the human TNFα antibody, or an antigen-binding portion thereof, is adalimumab.

In one embodiment, treatment of psoriatic arthritis is achieved by administering a TNFα inhibitor to a subject in accordance with a biweekly dosing regimen. Biweekly dosing regimens can be used to treat disorders in which TNFα activity is detrimental, and are further described in U.S. application Ser. No. 10/163,657 (US 20030235585), incorporated by reference herein.

In one embodiment, the invention provides a method of treating psoriatic arthritis in a subject comprising administering a human TNFα antibody, or antigen-binding portion thereof, e.g., adalimumab, to the subject at week 0 on a biweekly dosing regimen. In one embodiment, the human TNFα antibody, or antigen-binding portion thereof, is administered subcutaneously. In one embodiment, psoriatic arthritis is treated by administering a human TNFα antibody, or antigen-binding portion thereof, on biweekly dosing regimen for at least about 12, 24, 36 or 48 weeks.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Dosage regimens described herein may be adjusted to provide the optimum desired response, e.g., maintaining remission of psoriatic arthritis, in consideration of the teachings herein. It is to be noted that dosage values may vary with the type and severity of Psoriatic arthritis. It is to be further understood that for any particular subject, specific dosage regimens may be adjusted over time according to the combination of the teachings herein, the individual need, and/or professional judgment of the person administering or supervising the administration of the compositions. Furthermore, dosage amounts and ranges set forth herein are exemplary only and are not intended to limit the scope or practice of the claimed invention.

### Subpopulations

The invention provides uses and methods for treating certain subpopulations of psoriatic arthritis patients with a TNFα inhibitor. Also included in the invention are methods for determining whether a TNFα inhibitor, e.g., a TNFα antibody, or antigen-binding portion thereof, is effective for treating a certain subpopulation of PsA patients. Thus, the invention also includes a method of treating a subject who is a member of a subpopulation of PsA patients with a TNFα inhibitor which has been identified as being an effective TNFα inhibitor for the treatment of the given subpopulation.

In one embodiment, the invention also provides a method of treating a subject having certain types of PsA, including, but not limited to, moderate to severe PsA.

The invention also includes a method for treating a subject having PsA who has a certain extent of psoriasis. In one embodiment, the invention provides a method for treating a subject having PsA who has a body surface area (BSA) of <3%. In another embodiment, the invention provides a method for treating a subject having PsA who has a BSA of ≧3%. BSA refers to the percentage of body surface area affected by psoriasis

Traditional interventions for moderate to severe PsA have included nonsteroidal anti-inflammatory drugs (NSAIDs) and nonbiologic disease-modifying antirheumatic drugs (DMARDs). Certain subpopulations of PsA have been found, as described in the examples provided below, to not adequately respond to these traditional drugs.

In one embodiment, the invention provides a method for treating a subpopulation of psoriatic arthritis patients who have failed disease modifying anti-rheumatic drug (DMARDs) therapy, e.g., methotrexate, for the treatment of psoriatic arthritis. In certain instances, some patients who are administered a DMARD for the treatment of psoriatic arthritis have subtherapeutic responses to such treatment. In one embodiment, the invention provides use of a TNFα inhibitor in the manufacture of a medicament for treatment of psoriatic arthritis in a subject who has had a subtherapeutic response to a DMARD. In one embodiment, the invention provides an article of manufacture comprising adalimumab and a package insert, wherein the package insert indicates that adalimumab may be used to treat psoriatic arthritis in patients who have had an inadequate response to conventional DMARD therapy.

In one embodiment, the invention provides a method for treating a human subject having psoriatic arthritis (PsA) who has failed Non-Steroidal Anti-Inflammatory Drug (NSAID) therapy comprising administering to the subject a TNFα inhibitor, such that PsA is treated.

The invention further includes methods of treating any of the subpopulations of patients who respond to TNFα inhibitor treatment for PsA described in the examples set forth below.

### Articles of Manufacture

The invention also provides a packaged pharmaceutical composition wherein the TNFα inhibitor, e.g., TNFα antibody, is packaged within a kit or an article of manufacture. The kit or article of manufacture of the invention contains materials useful for the treatment, prevention and/or diagnosis of psoriatic arthritis. The kit or article of manufacture comprises a container and a label or package insert or printed material on or associated with the container which provides information regarding use of the TNFα inhibitor, e.g., a TNFα antibody, for the treatment of psoriatic arthritis.

A kit or an article of manufacture refers to a packaged product comprising components with which to administer a TNFα inhibitor for treatment of psoriatic arthritis. The kit preferably comprises a box or container that holds the components of the kit. The box or container is affixed with a label or a Food and Drug Administration approved label, including a protocol for administering the TNFα inhibitor. The box or container holds components of the invention which are preferably contained within plastic, polyethylene, polypropylene, ethylene, or propylene vessels. The vessels can be capped-tubes or bottles. The kit can also include instructions for administering the TNFα antibody of the invention. In one embodiment the kit of the invention includes the formulation comprising the human antibody adalimumab (or D2E7), as described in PCT/IB03/04502 and U.S. application Ser. No. 10/222,140, incorporated by reference herein.

The term “package insert” is used to refer to instructions customarily included in commercial packages of therapeutic products, that contain information about the indications, usage, dosage, administration, contraindications and/or warnings concerning the use of such therapeutic products.

In one embodiment, the article of manufacture of the invention comprises (a) a first container with a composition contained therein, wherein the composition comprises a TNFα antibody; and (b) a package insert indicating that the TNFα antibody may be used for reducing signs and symptoms and treatment of psoriatic arthritis. In a preferred embodiment, the label or package insert indicates that the TNFα inhibitor, e.g., a TNFα antibody, is used for treatment of psoriatic arthritis.

Suitable containers for the TNFα inhibitor, e.g., a TNFα antibody, include, for example, bottles, vials, syringes, pens, etc. The containers may be formed from a variety of materials such as glass or plastic. The container holds a composition which is by itself or when combined with another composition effective for treating, preventing and/or diagnosing the condition and may have a sterile access port.

In one embodiment, the article of manufacture comprises a TNFα inhibitor, e.g., a TNFα antibody, and a label which indicates to a subject who will be administering the TNFα inhibitor about using the TNFα inhibitor for the treatment of psoriatic arthritis. The label may be anywhere within or on the article of manufacture. In one embodiment, the article of manufacture comprises a container, such as a box, which comprises the TNFα inhibitor and a package insert or label providing information pertaining to use of the TNFα inhibitor for the treatment of psoriatic arthritis. In another embodiment, the information is printed on a label which is on the outside of the article of manufacture, in a position which is visible to prospective purchasers.

In one embodiment, the package insert of the invention informs a reader, including a subject, e.g., a purchaser, who will be administering the TNFα inhibitor for treatment, that the TNFα inhibitor, e.g., a TNFα antibody such as adalimumab, is an indicated treatment of psoriatic arthritis, including of moderately to severely active disease in adult patients.

In one embodiment, the package insert describes certain patient populations who may respond favorably to the TNFα inhibitor within the article of manufacture. For example, the package insert may indicate that the TNFα antibody, e.g., adalimumab, may be used to treat psoriatic arthritis in patients who have had an inadequate response to conventional therapy, .e.g., DMARDs.

In one embodiment, the invention provides an article of manufacture comprising a packaging material; a human TNFα antibody, or antigen-binding portion thereof; and a label or package insert contained within the packaging material indicating that a human TNFα antibody, or antigen-binding portion thereof, may be used to reduce signs and symptoms of active arthritis in patients having PsA; that a human TNFα antibody, or antigen-binding portion thereof, may be used to inhibit the progression of structural damage in patients having PsA; and/or that a human TNFα antibody, or antigen-binding portion thereof, may be used to improve physical function in patients having PsA. In still another embodiment, the invention includes a package insert which describes the a human TNFα antibody, or antigen-binding portion thereof, reduces signs and symptoms of active arthritis, inhibits the progression of structural damage, and improved physical function when used for treatment of PsA.

In one embodiment, the package insert of the invention describes certain therapeutic benefits of the TNFα antibody, e.g., adalimumab, including specific symptoms of psoriatic arthritis which may be reduced by using the TNFα antibody, e.g., adalimumab. The package insert of the invention may also indicate that adalimumab helps reduce the signs and symptoms of immune diseases, including rheumatoid and psoriatic arthritis (pain and swollen joints), ankylosing spondylitis (morning stiffness and back pain), and Psoriatic arthritis (abdominal pain and diarrhea).

In another embodiment, the package insert of the invention describes the dose and administration of adalimumab, for the treatment of psoriatic arthritis. The label may indicate that the initiation of therapy includes a biweekly 40 mg subcutaneous dose. In another embodiment, the package insert of the invention indicates that adalimumab is administered by subcutaneous injection.

In another embodiment, the label of the invention indicates that the recommended TNFα inhibitor, e.g., a TNFα antibody such as adalimumab, dose regimen for adult patients with psoriatic arthritis is 40 mg at week 0, followed by 40 mg every other week.

The package insert of the invention may also provide information to subjects who will be receiving adalimumab regarding combination uses for both safety and efficacy purposes. The package insert of the invention may contain warnings and precautions regarding the use of the TNFα inhibitor, e.g., a TNFα antibody such as adalimumab. For example, the package insert may identify any of the adverse events (AEs) associated with the TNFα inhibitor when used for treatment of PsA, including those AEs described in the examples below.

The label of the invention may contain information regarding the use of the TNFα inhibitor, e.g., a TNFα antibody such as adalimumab, in clinical studies for psoriatic arthritis. In one embodiment, the label of the invention describes the studies described herein as the Examples, either as a whole or in portion.

In one embodiment of the invention, the kit comprises a TNFα inhibitor, such as an antibody, a second pharmaceutical composition comprising an additional therapeutic agent, and instructions for administration of both agents for the treatment of psoriatic arthritis. The instructions may describe how, e.g., subcutaneously, and when, e.g., at week 0, week 2, and biweekly thereafter, doses of TNFα antibody and/or the additional therapeutic agent shall be administered to a subject for treatment.

Another aspect of the invention pertains to kits containing a pharmaceutical composition comprising an anti-TNFα antibody and a pharmaceutically acceptable carrier and one or more additional pharmaceutical compositions each comprising a drug useful for treating a TNFα related disorder and a pharmaceutically acceptable carrier. Alternatively, the kit comprises a single pharmaceutical composition comprising an anti-TNFα antibody, one or more drugs useful for treating a TNFα related disorder and a pharmaceutically acceptable carrier. The kits further contain instructions for dosing of the pharmaceutical compositions for the treatment of a TNFα related disorder.

The package or kit alternatively may contain the TNFα inhibitor and it may be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### Additional Therapeutic Agents

Methods, uses, and compositions of the invention also include combinations of TNFα inhibitors, including antibodies, and other therapeutic agents. TNFα inhibitors, including antibodies, or antigen binding portions thereof, can be used alone or in combination with additional agents to treat PsA. It should be understood that antibodies, or antigen binding portion thereof, can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent that imparts a beneficial attribute to the therapeutic composition e.g., an agent which affects the viscosity of the composition.

It should further be understood that the combinations which are to be included within this invention are those combinations useful for their intended purpose. The agents set forth below are illustrative for purposes and not intended to be limited. The combinations, which are part of this invention, can be the antibodies of the present invention and at least one additional agent selected from the lists below. The combination can also include more than one additional agent, e.g., two or three additional agents if the combination is such that the formed composition can perform its intended function.

TNFα inhibitors described herein may be used in combination with additional therapeutic agents such as a Disease Modifying Anti-Rheumatic Drug (DMARD) or a Nonsteroidal Antiinflammatory Drug (NSAID) or a steroid or any combination thereof. Preferred examples of a DMARD are hydroxychloroquine, leflunomide, methotrexate, parenteral gold, oral gold and sulfasalazine. Preferred examples of non-steroidal anti-inflammatory drug(s) also referred to as NSAIDS include drugs like ibuprofen. Other preferred combinations are corticosteroids including prednisolone; the well known side effects of steroid use can be reduced or even eliminated by tapering the steroid dose required when treating patients in combination with the anti-TNFα antibodies of this invention. Non-limiting examples of therapeutic agents for rheumatoid arthritis with which an antibody, or antibody portion, of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, IL-23, interferons, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists/inhibitors such as soluble p55 or p75 TNF receptors, derivatives, thereof, (p75TNFR1gG (Enbrel™) or p55TNFR1gG (Lenercept), chimeric, humanized or human TNF antibodies, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (Humira® Abbott Laboratories, a human anti-TNF mAb, described in U.S. Pat. No. 6,090,382 as D2E7). Additional TNF antibodies which can be used in the invention are described in U.S. Pat. Nos. 6,593,458; 6,498,237; 6,451,983; and 6,448,380, each of which is incorporated by reference herein. Other combinations including TNFα converting enzyme (TACE) inhibitors; IL-1 inhibitors (Interleukin-1-converting enzyme inhibitors, IL-1RA etc.) may be effective for the same reason. Other combinations include the IL-6 antibody tocilizumab (Actemra). Other preferred combinations include Interleukin 11. Yet another preferred combination are other key players of the autoimmune response which may act parallel to, dependent on or in concert with TNFα function; especially preferred are IL-18 antagonists including IL-18 antibodies or soluble IL-18 receptors, or IL-18 binding proteins. It has been shown that TNFα and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

The TNFα inhibitors, including antibodies, or antigen binding portions thereof, used in the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-1β converting enzyme inhibitors, TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), tocilizumab (Actemra), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SC10-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine.

Non-limiting examples of therapeutic agents for psoriatic arthritis with which TNFα inhibitor, such as an antibody, or antibody portion, can be combined include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, efalizumab.

Non-limiting examples of therapeutic agents for psoriatic arthritis with which TNFα inhibitor, such as an antibody, or antibody portion, can be combined also include alemtuzumab, dronabinol, Unimed, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists, IL-4 agonists.

In one embodiment, the methods and compositions of the invention provide a combination use of a TNFα antibody, e.g., adalimumab, and a DMARD, e.g., methotrexate.

### IV. Efficacy of TNFα Inhibitor

The invention also provides methods for determining whether a TNFα inhibitor is effective at treating psoriatic arthritis in a subject. Such methods may be used to determine the efficacy of a TNFα inhibitor, including those which are unknown or unconfirmed to have such efficacy. Using the methods described herein, effective TNFα inhibitors may be determined or confirmed, and, subsequently, used in the method of treating psoriatic arthritis.

In one embodiment, the invention provides a method for determining the efficacy of a TNFα inhibitor, including a human TNFα antibody, for treatment of psoriatic arthritis in a subject using the American College of Rheumatology (ACR) preliminary criteria for improvement. ACR criteria measures improvement in tender or swollen joint counts and improvement in three of the following five parameters: acute phase reactant (such as sedimentation rate); patient assessment; physician assessment; pain scale; and disability/functional questionnaire. ACR criteria is indicated as ACR 20 (a 20 percent improvement in tender or swollen joint counts as well as 20 percent improvement in three of the other five criteria), ACR 50 (a 50 percent improvement in tender or swollen joint counts as well as 50 percent improvement in three of the other five criteria), and ACR 70 (a 70 percent improvement in tender or swollen joint counts as well as 70 percent improvement in three of the other five criteria) (see Felson et al. Arthritis Rheum 1995; 38:727-35).

The efficacy of a TNFα inhibitor for treatment of psoriatic arthritis in a patient population who has psoriatic arthritis may be evaluated by determining the percentage of the patient population in whom an ACR20, ACR50 or ACR 70 response has been achieved following administration of the TNFα inhibitor.

In one aspect, the invention provides a method of determining the efficacy of a TNFα inhibitor for treating psoriatic arthritis in a subject comprising determining a an ACR20 response of a patient population having psoriatic arthritis and who was administered the TNFα inhibitor, wherein a an ACR20 response in at least about 39% of the patient population indicates that the TNFα inhibitor is an effective TNFα inhibitor for the treatment of psoriatic arthritis in a subject. In one embodiment, the method further comprises administering the effective TNFα inhibitor to a subject to treat psoriatic arthritis. The invention provides a method of treatment of psoriatic arthritis in a subject comprising administering an effective amount of a TNFα inhibitor to the subject such that the subject is treated, wherein the effective amount of the TNFα inhibitor was previously identified as achieving an ACR20 response in at least about, e.g., 39%, of a patient population having psoriatic arthritis. In one embodiment, an ACR20 response in at least about 39% of the patient population indicates that the TNFα inhibitor is an effective TNFα inhibitor for the treatment of psoriatic arthritis in a subject. In one embodiment, an ACR20 response in at least about 40% of the patient population indicates that the TNFα inhibitor is an effective TNFα inhibitor for the treatment of psoriatic arthritis in a subject. In one embodiment, an ACR20 response in at least about 45% of the patient population indicates that the TNFα inhibitor is an effective TNFα inhibitor for the treatment of psoriatic arthritis in a subject. In one embodiment, an ACR20 response in at least about 50% of the patient population indicates that the TNFα inhibitor is an effective TNFα inhibitor for the treatment of psoriatic arthritis in a subject. In one embodiment, an ACR20 response in at least about 55% of the patient population indicates that the TNFα inhibitor is an effective TNFα inhibitor for the treatment of psoriatic arthritis in a subject. In one embodiment, an ACR20 response in at least about 57% of the patient population indicates that the TNFα inhibitor is an effective TNFα inhibitor for the treatment of psoriatic arthritis in a subject. In one embodiment, an ACR20 response in at least about 60% of the patient population indicates that the TNFα inhibitor is an effective TNFα inhibitor for the treatment of psoriatic arthritis in a subject. In one embodiment, an ACR20 response in at least about 61% of the patient population indicates that the TNFα inhibitor is an effective TNFα inhibitor for the treatment of psoriatic arthritis in a subject. In one embodiment, an ACR20 response in at least about 64% of the patient population indicates that the TNFα inhibitor is an effective TNFα inhibitor for the treatment of psoriatic arthritis in a subject. Numbers intermediate to the above recited percentages, e.g., 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, as well as all other numbers recited herein, are also intended to be part of this invention. Ranges of values using a combination of any of the above recited values as upper and/or lower limits are intended to be included in the scope of the invention. For example, in one embodiment, an ACR50 response in at least about 39% to at least about 60% of the patient population indicates that the human TNFα antibody is an effective human TNFα antibody for the treatment of psoriatic arthritis in a subject.

In some aspects, the invention provides a method of determining the efficacy of a human TNFα antibody for treating psoriatic arthritis in a subject comprising determining an ACR50 response of a patient population having psoriatic arthritis and who was administered the human TNFα antibody, wherein an ACR50 response in at least about 25% of the patient population indicates that the human TNFα antibody is an effective human TNFα antibody for the treatment of psoriatic arthritis in a subject. In one embodiment, the method further comprises administering the effective TNFα inhibitor to a subject to treat psoriatic arthritis. The invention provides a method of treatment of psoriatic arthritis in a subject comprising administering an effective amount of a TNFα inhibitor to the subject such that the subject is treated, wherein the effective amount of the TNFα inhibitor was previously identified as achieving an ACR50 response in at least about, e.g, 30%, of a patient population having psoriatic arthritis.

In one embodiment, an ACR50 response in at least about 35% of the patient population indicates that the human TNFα antibody is an effective human TNFα antibody for the treatment of psoriatic arthritis in a subject. In one embodiment, an ACR50 response in at least about 36% of the patient population indicates that the human TNFα antibody is an effective human TNFα antibody for the treatment of psoriatic arthritis in a subject. In one embodiment, an ACR50 response in at least about 39% of the patient population indicates that the human TNFα antibody is an effective human TNFα antibody for the treatment of psoriatic arthritis in a subject. In one embodiment, an ACR50 response in at least about 42% of the patient population indicates that the human TNFα antibody is an effective human TNFα antibody for the treatment of psoriatic arthritis in a subject. In one embodiment, an ACR50 response in at least about 43% of the patient population indicates that the human TNFα antibody is an effective human TNFα antibody for the treatment of psoriatic arthritis in a subject. In one embodiment, an ACR50 response in at least about 46% of the patient population indicates that the human TNFα antibody is an effective human TNFα antibody for the treatment of psoriatic arthritis in a subject. Numbers intermediate to the above recited percentages, e.g., 25%, 26%, 27%, 28%, 29%, 30%, 31%, 32%, 33%, 34%, 35%, 36%, 37%. 38%. 39%, 40%, 41%, 42%, 43%, 44%, 45%, 46%, as well as all other numbers recited herein, are also intended to be part of this invention. Ranges of values using a combination of any of the above recited values as upper and/or lower limits are intended to be included in the scope of the invention. For example, in one embodiment, an ACR50 response in at least about 25% to at least about 46% of the patient population indicates that the human TNFα antibody is an effective human TNFα antibody for the treatment of psoriatic arthritis in a subject.

In some aspects, the invention provides a method of determining the efficacy of a human TNFα antibody for treating psoriatic arthritis in a subject comprising determining an ACR70 response of a patient population having psoriatic arthritis and who was administered the human TNFα antibody, wherein an ACR70 response in at least about 14% of the patient population indicates that the human TNFα antibody is an effective human TNFα antibody for the treatment of psoriatic arthritis in a subject. In one embodiment, the method further comprises administering the effective TNFα inhibitor to a subject to treat psoriatic arthritis. The invention provides a method of treatment of psoriatic arthritis in a subject comprising administering an effective amount of a TNFα inhibitor to the subject such that the subject is treated, wherein the effective amount of the TNFα inhibitor was previously identified as achieving an ACR70 response in at least about, e.g., 14% of a patient population having psoriatic arthritis.

In one embodiment, an ACR70 response in at least about 14% of the patient population indicates that the human TNFα antibody is an effective human TNFα antibody for the treatment of psoriatic arthritis in a subject. In one embodiment, an ACR70 response in at least about 20% of the patient population indicates that the human TNFα antibody is an effective human TNFα antibody for the treatment of psoriatic arthritis in a subject. In one embodiment, an ACR70 response in at least about 22% of the patient population indicates that the human TNFα antibody is an effective human TNFα antibody for the treatment of psoriatic arthritis in a subject. In one embodiment, an ACR70 response in at least about 23% of the patient population indicates that the human TNFα antibody is an effective human TNFα antibody for the treatment of psoriatic arthritis in a subject. In one embodiment, an ACR70 response in at least about 25% of the patient population indicates that the human TNFα antibody is an effective human TNFα antibody for the treatment of psoriatic arthritis in a subject. In one embodiment, an ACR70 response in at least about 27% of the patient population indicates that the human TNFα antibody is an effective human TNFα antibody for the treatment of psoriatic arthritis in a subject. In one embodiment, an ACR70 response in at least about 31% of the patient population indicates that the human TNFα antibody is an effective human TNFα antibody for the treatment of psoriatic arthritis in a subject. Numbers intermediate to the above recited percentages, e.g., 14, 15, 16, 17, 18, 19, 20, 21, 22, 23, 24, 25, 26, 27, 28, 29, 30, 31%, as well as all other numbers recited herein, are also intended to be part of this invention. Ranges of values using a combination of any of the above recited values as upper and/or lower limits are intended to be included in the scope of the invention. For example, in one embodiment, an ACR70 response in at least about 14% to at least about 31% of the patient population indicates that the human TNFα antibody is an effective human TNFα antibody for the treatment of psoriatic arthritis in a subject.

The invention provides a method for determining the efficacy of a TNFα inhibitor, including a human TNFα antibody, for treatment of psoriatic arthritis in a subject, using the Psoriasis Area and Severity Index (PASI). The PASI is used by dermatologists to assess psoriasis disease intensity. This index is based on the quantitative assessment of three typical signs of psoriatic lesions: erythema, infiltration, and desquamation, combined with the skin surface area involvement (see Fredriksson T, et al. Dermatologica 1978; 157: 238-41). PASI is indicated as PASI50 (a 50 percent improvement in PASI from baseline), PASI75 (a 75 percent improvement in PASI from baseline), PASI90 (a 90 percent improvement in PASI from baseline), and PASI100 (a 100 percent improvement in PASI from baseline).

The efficacy of a TNFα inhibitor for treatment of psoriatic arthritis in a patient population who has psoriatic arthritis, may be evaluated by determining the percentage of the patient population in whom a PASI50, PASI75, PASI90, or PASI100 response has been achieved following administration of the TNFα inhibitor.

In some aspects, the invention provides a method of determining the efficacy of a TNFα inhibitor for treating psoriatic arthritis in a subject comprising determining a PASI50 response of a patient population having psoriatic arthritis and who was administered the TNFα inhibitor, wherein a PASI50 response in at least about 70% of the patient population indicates that the TNFα inhibitor is an effective TNFα inhibitor for the treatment of psoriatic arthritis in a subject. In one embodiment, the method further comprises administering the effective TNFα inhibitor to a subject to treat psoriatic arthritis. In some aspects, the present invention provides a method of treating psoriatic arthritis in a subject comprising administering an effective TNFα inhibitor to the subject such that psoriatic arthritis is treated, wherein the effective TNFα inhibitor was previously identified as achieving a PASI50 response in at least about 70% of the patient population.

In one embodiment, a PASI50 response in at least about 70% of the patient population indicates that the TNFα inhibitor is an effective TNFα inhibitor for the treatment of psoriatic arthritis in a subject. In one embodiment, a PASI50 response in at least about 72% of the patient population indicates that the TNFα inhibitor is an effective TNFα inhibitor for the treatment of psoriatic arthritis in a subject. In one embodiment, a PASI50 response in at least about 73% of the patient population indicates that the TNFα inhibitor is an effective TNFα inhibitor for the treatment of psoriatic arthritis in a subject. In one embodiment, a PASI50 response in at least about 75% of the patient population indicates that the TNFα inhibitor is an effective TNFα inhibitor for the treatment of psoriatic arthritis in a subject. In one embodiment, a PASI50 response in at least about 76% of the patient population indicates that the TNFα inhibitor is an effective TNFα inhibitor for the treatment of psoriatic arthritis in a subject. Numbers intermediate to the above recited percentages, e.g., 70, 71, 72, 73, 74, 75, 76%, as well as all other numbers recited herein, are also intended to be part of this invention. Ranges of values using a combination of any of the above recited values as upper and/or lower limits are intended to be included in the scope of the invention. For example, in one embodiment, a PASI50 response in at least about 70% to at least about 76% of the patient population indicates that the TNFα inhibitor is an effective TNFα inhibitor for the treatment of psoriatic arthritis in a subject.

In some aspects, the invention provides a method of determining the efficacy of a human TNFα antibody for treating psoriatic arthritis in a subject comprising determining a PASI75 response of a patient population having psoriatic arthritis and who was administered the human TNFα antibody, wherein a PASI75 response in at least about 40% of the patient population indicates that the human TNFα antibody is an effective human TNFα antibody for the treatment of psoriatic arthritis in a subject. IN one embodiment, the method further comprises administering the effective human TNFα antibody to a subject to treat psoriatic arthritis. In some aspects, a method of treating psoriatic arthritis in a subject comprising administering an effective human TNFα antibody to the subject such that psoriatic arthritis is treated, wherein the effective human TNFα antibody was previously identified as achieving a PASI75 response in at least about 40% of the patient population.

In one embodiment, a PASI75 response in at least about 40% of the patient population indicates that the human TNFα antibody is an effective human TNFα antibody for the treatment of psoriatic arthritis in a subject. In one embodiment, a PASI75 response in at least about 45% of the patient population indicates that the human TNFα antibody is an effective human TNFα antibody for the treatment of psoriatic arthritis in a subject. In one embodiment, a PASI75 response in at least about 50% of the patient population indicates that the human TNFα antibody is an effective human TNFα antibody for the treatment of psoriatic arthritis in a subject. In one embodiment, a PASI75 response in at least about 55% of the patient population indicates that the human TNFα antibody is an effective human TNFα antibody for the treatment of psoriatic arthritis in a subject. In one embodiment, a PASI75 response in at least about 59% of the patient population indicates that the human TNFα antibody is an effective human TNFα antibody for the treatment of psoriatic arthritis in a subject. Numbers intermediate to the above recited percentages, e.g., 40, 41, 42, 43, 44, 45, 46, 47, 48, 49, 50, 51, 52, 53, 54, 55, 56, 57, 58, 59%, as well as all other numbers recited herein, are also intended to be part of this invention. Ranges of values using a combination of any of the above recited values as upper and/or lower limits are intended to be included in the scope of the invention. For example, in one embodiment, a PASI75 response in at least about 40% to at least about 58% of the patient population indicates that the human TNFα antibody is an effective human TNFα antibody for the treatment of psoriatic arthritis in a subject.

In some aspects, the invention provides a method of determining the efficacy of a TNFα inhibitor for treating psoriatic arthritis in a subject comprising determining a PASI90 response of a patient population having psoriatic arthritis and who was administered the TNFα inhibitor, wherein a PASI90 response in at least about 25% of the patient population indicates that the TNFα inhibitor is an effective TNFα inhibitor for the treatment of psoriatic arthritis in a subject. In one embodiment, the invention further comprises administering the effective TNFα inhibitor to a subject to treat psoriatic arthritis. In some aspects, the invention provides a method of treating psoriatic arthritis in a subject comprising administering an effective TNFα inhibitor to the subject such that psoriatic arthritis is treated, wherein the effective TNFα inhibitor was previously identified as achieving a PASI90 response in at least about 25% of the patient population.

In one embodiment, a PASI90 response in at least about 25% of the patient population indicates that the TNFα inhibitor is an effective TNFα inhibitor for the treatment of psoriatic arthritis in a subject. In one embodiment, a PASI90 response in at least about 30% of the patient population indicates that the TNFα inhibitor is an effective TNFα inhibitor for the treatment of psoriatic arthritis in a subject. In one embodiment, a PASI90 response in at least about 35% of the patient population indicates that the TNFα inhibitor is an effective TNFα inhibitor for the treatment of psoriatic arthritis in a subject. In one embodiment, a PASI90 response in at least about 40% of the patient population indicates that the TNFα inhibitor is an effective TNFα inhibitor for the treatment of psoriatic arthritis in a subject. In one embodiment, a PASI90 response in at least about 42% of the patient population indicates that the TNFα inhibitor is an effective TNFα inhibitor for the treatment of psoriatic arthritis in a subject. Numbers intermediate to the above recited percentages, e.g., 25, 26, 27, 28, 29, 30, 31, 32, 33, 34, 35, 36, 37, 38, 39, 40, 41, 42%, as well as all other numbers recited herein, are also intended to be part of this invention. Ranges of values using a combination of any of the above recited values as upper and/or lower limits are intended to be included in the scope of the invention. For example, in one embodiment, a PASI90 response in at least about 26% to at least about 41% of the patient population indicates that the TNFα inhibitor is an effective TNFα inhibitor for the treatment of psoriatic arthritis in a subject

The invention provides a method for determining the efficacy of a TNFα inhibitor, including a human TNFα antibody, for treatment of psoriatic arthritis in a subject, using the Physician's Global Assessment scale (PGA). PGA is used to assess psoriasis activity and follow clinical response to treatment. It is a score that summarizes the overall quality (erythema, scaling and thickness) and extent of plaques relative to the baseline assessment. A patient's response is rated as worse, poor (0-24%), fair (25-49%), good (50-74%), excellent (75-99%), or cleared (100%) (see van der Kerkhof P. Br J Dermatol 1997; 137:661-662).

The efficacy of a TNFα inhibitor for treatment of psoriatic arthritis in a patient population who has psoriatic arthritis, can be evaluated by determining the percentage of the patient population in whom a PGA of “Clear” or “Almost Clear” has been achieved following administration of the TNFα inhibitor, including a human TNFα antibody.

In some aspects, the invention provides a method of determining the efficacy of a human TNFα antibody for treating psoriatic arthritis in a subject comprising determining a PGA response of “Clear” or “Almost Clear,” of a patient population having psoriatic arthritis and who was administered the human TNFα antibody, wherein a PGA response of “Clear” or “Almost Clear,” in at least about 40% of the patient population indicates that the human TNFα antibody is an effective human TNFα antibody for the treatment of psoriatic arthritis in a subject. In one embodiment, the invention further comprises administering the effective human TNFα antibody to a subject to treat psoriatic arthritis. In some aspects, the invention provides a method of treating psoriatic arthritis in a subject comprising administering an effective human TNFα antibody to the subject such that psoriatic arthritis is treated, wherein the effective human TNFα antibody was previously identified as achieving a PGA response of “Clear” or “Almost Clear,” in at least about 40% of the patient population.

In one embodiment, a PGA response of “Clear” or “Almost Clear,” in at least about 45% of the patient population indicates that the human TNFα antibody is an effective human TNFα antibody for the treatment of psoriatic arthritis in a subject. In one embodiment, a PGA response of “Clear” or “Almost Clear,” in at least about 50% of the patient population indicates that the human TNFα antibody is an effective human TNFα antibody for the treatment of psoriatic arthritis in a subject. In one embodiment, a PGA response of “Clear” or “Almost Clear,” in at least about 55% of the patient population indicates that the human TNFα antibody is an effective human TNFα antibody for the treatment of psoriatic arthritis in a subject. In one embodiment, a PGA response of “Clear” or “Almost Clear,” in at least about 60% of the patient population indicates that the human TNFα antibody is an effective human TNFα antibody for the treatment of psoriatic arthritis in a subject. In one embodiment, a PGA response of “Clear” or “Almost Clear,” in at least about 67% of the patient population indicates that the human TNFα antibody is an effective human TNFα antibody for the treatment of psoriatic arthritis in a subject. Numbers intermediate to the above recited percentages, e.g., 45, 46, 47, 48, 49, 50, 51, 52, 53, 54, 55, 56, 57, 58, 59, 60, 61, 62, 63, 64, 65, 66, 67%, as well as all other numbers recited herein, are also intended to be part of this invention. Ranges of values using a combination of any of the above recited values as upper and/or lower limits are intended to be included in the scope of the invention. For example, in one embodiment a PGA response of “Clear” or “Almost Clear,” in at least about 46% to at least about 59% of the patient population indicates that the human TNFα antibody is an effective human TNFα antibody for the treatment of psoriatic arthritis in a subject.

Additional measures can be used to evaluate the efficacy of a TNFα inhibitor for treatment of psoriatic arthritis, or improvement in the quality of life (QOL) and physical function in a patient population who has psoriatic arthritis, following administration of the TNFα inhibitor, including a human TNFα antibody, or antigen binding fragment thereof. Examples of QOL measures include the Short-Form 36 (SF-36), a broad measure of physical and mental domains which has been used and validated in many diseases, and the Dermatology Life Quality Index (DLQI).

In one embodiment, a Health Assessment Questionnaire (HAQ) is used to evaluate the efficacy of a TNFα inhibitor for treatment of psoriatic arthritis in a patient population who has psoriatic arthritis. The HAQ is a standardized disability questionnaire that was initially developed for use in rheumatoid arthritis. The HAQ-DI assesses the difficulty a patient has accomplishing tasks in eight functional areas (dressing, arising, eating, walking, hygiene, reaching, gripping and other activities of daily living). A high HAQ score has been shown to be a strong predictor of morbidity and mortality in RA, and low HAQ scores are predictive of better outcomes (see Fries J F, et al. Arthritis Rheum 1980; 23:137-45).

In one embodiment, the invention provides a method of determining the efficacy of a TNFα inhibitor for treating psoriatic arthritis in a subject comprising determining a Health Assessment Questionnaire (HAQ) response of a patient population having psoriatic arthritis and who was administered the TNFα inhibitor, wherein an average decrease of about 0.3 in the HAQ score of the patient population indicates that the TNFα inhibitor is an effective TNFα inhibitor for the treatment of psoriatic arthritis in a subject. In one embodiment, an average decrease of about 0.4 in the HAQ score of the patient population indicates that the TNFα inhibitor is an effective TNFα inhibitor for the treatment of psoriatic arthritis in a subject. In one embodiment, an average decrease of about 0.5 in the HAQ score of the patient population indicates that the TNFα inhibitor is an effective TNFα inhibitor for the treatment of psoriatic arthritis in a subject. The invention further comprises administering the effective TNFα inhibitor to a subject to treat psoriatic arthritis. The invention further provides a method of treating psoriatic arthritis in a subject comprising administering an effective TNFα inhibitor to the subject such that psoriatic arthritis (PsA) is treated, wherein the effective TNFα inhibitor was previously identified as decreasing the HAQ average score, for example between about 0.3 and 0.5 in a patient population having PsA. HAQ score decreases may be determined according to a comparison to a baseline score.

A number of measures of fatigue have been developed as well which may be used to determine the efficacy of a TNFα inhibitor for treating PsA. Fatigue is an important domain to PsA patients; even in patients without evident clinical psoriasis, fatigue is often overlooked by assessors, yet is capable of significant improvement with newer therapies. In one embodiment, the Functional Assessment of Chronic Illness Therapy (FACIT) can be used to evaluate the efficacy of a TNFα inhibitor for the treatment of psoriatic arthritis in a patient population who has psoriatic arthritis.

It should be noted that the Examples provided herein represent different methods of determining the efficacy of a TNFα inhibitor, such as a human TNFα antibody, or antigen-binding portion thereof. As such, data and results described in the Examples section which shows efficacy of a TNFα inhibitor, e.g., treatment of psoriatic arthritis, are included in the methods of determining efficacy of the invention.

Time points for determining efficacy will be understood by those of skill in the art to depend on the type of efficacy being determined. In one embodiment, measurements in scores, e.g., ACR20/50/70 response, or PASI50/75/90 response, may be measured against a subject's baseline score. Generally, a baseline refers to a measurement or score of a patient before treatment, i.e. week 0. Other time points may also be included as a starting point in determining efficacy, however.

Patient populations described in the methods of the invention are generally selected based on common characteristics, such as, but not limited to, subjects diagnosed with psoriatic arthritis. Such a patient population would be appropriate for determining the efficacy of the TNFα inhibitor for treating psoriatic arthritis in the given patient population. In one embodiment, the patient population is an adult population, .e.g, older than 17 years of age or older than 18 years of age.

In one embodiment, the methods of the invention for determining whether a TNFα inhibitor is an effective TNFα inhibitor, include determining changes, improvements, measurements, etc., in psoriatic arthritis using appropriate indices known in the art, e.g., ACR, PASI, PGA, HAQ, DLQI, FACIT-F from a patient population who has already been administered the TNFα inhibitor. Such a patient population may be pre-selected according to common characteristics, .e.g., psoriatic arthritis, loss of response to DMARDs, and may have already been given the TNFα inhibitor. Administration of the TNFα inhibitor may or may not be performed by the same person of ordinary skill who is determining the efficacy of the TNFα inhibitor in accordance with the teachings of the specification.

In one embodiment, the methods of the invention comprise administering the TNFα inhibitor to the subjects of a patient population and determining the efficacy of the TNFα inhibitor by determining changes, improvements, measurements, etc., using psoriatic arthritis indices known in the art, in the patient population in comparison to the Examples set forth below. For example, in one embodiment the invention includes a method for determining the efficacy of a TNFα inhibitor for the treatment of psoriatic arthritis comprising administering the TNFα inhibitor to a preselected patient population having psoriatic arthritis; and determining the effectiveness of the TNFα inhibitor by using a mean baseline ACR score of the patient population and a mean ACR20 score following administration of the TNFα inhibitor, wherein a ACR20 achieved in at least about 39% of the patient population indicates that the TNFα inhibitor is effective for the treatment of psoriatic arthritis.

The Examples and discoveries described herein are representative of a TNFα inhibitor, i.e., adalimumab, which is effective for treating psoriatic arthritis. As such, the studies and results described in the Examples section herein may be used as a guideline for determining the efficacy of a TNFα inhibitor, i.e., whether a TNFα inhibitor is an effective TNFα inhibitor for the treatment of psoriatic arthritis. In one embodiment, methods of determining efficacy described herein may be used to determine whether a TNFα inhibitor is bioequivalent to another TNFα inhibitor.

In one embodiment, the article of manufacture of the invention comprises instructions regarding how to determine the efficacy of the TNF inhibitor for the treatment of psoriatic arthritis. The present invention is further illustrated by the following examples which should not be construed as limiting in any way.

### EXAMPLES

### Example 1

### Determinants of Health State Utility in Patients with Psoriatic Arthritis

Quality of life (QoL) is an important indicator of therapeutic effectiveness. In addition to the assessment of patient limitations in daily activities, preferences around QoL are critical for health care assessments, including economic evaluations of treatment. Currently, there is only limited research into the main determinants of QoL in patients with psoriatic arthritis (PsA). To this end, health utilities, which measure patient preferences, were used to examine associations between clinical outcomes. Adalimumab is a fully human, anti-tumor necrosis factor monoclonal antibody under investigation for the treatment of PsA.

This study was conducted to assess the validity of a novel method to derive health utilities for PsA. In order to assess the validity of the novel method to derive health utilities for PsA, a pivotal, Phase III, randomized controlled trial (Study G) of adalimumab vs. placebo in the treatment of PsA was conducted. Data on patient-reported functional loss measured by the Health Assessment Questionnaire Disability Index (HAQ DI); physician's assessment of psoriasis severity from the Psoriasis Area Severity Index (PASI); tender and swollen joint counts (TJC, SJC); a general QoL questionnaire, SF-36; and age, sex, and disease duration were collected for patients at baseline and Weeks 12 and 24. To assess significant predictors of health-related utilities, the SF-6D, a utility measurement, was derived from responses to SF-36 using the Brazier algorithm. Multiple linear regressions using generalized estimating equations were employed to identify significant predictors of SF-6D. Endpoints related to skin and joint function were added to the model comparisons to determine their associations with the SF-6D.

Mean baseline characteristics for 313 patients included age=49 years, disease duration=9.5 years, TJC=24, SJC=14, HAQ-DI=1.0, and PASI=7.9. In addition, 44% of patients were female. As determined by multiple linear regression, significant independent predictors of PsA-related QoL (in descending order of importance) were: functional loss (HAQ-DI), severity of psoriasis (PASI), and TJC (all p<0.05). SJC was not a significant predictor of QoL in PsA.

In patients with PsA, the main determinants of QoL measured were degree of disease-related functional loss and severity of skin disease. In contrast to findings in rheumatoid arthritis, joint counts were of secondary importance. These findings have important implications for economic evaluations of new treatments for PsA. Additional details be found in Ann Rheum Dis 2005; 64(Suppl III):579, which is incorporated by reference herein.

### Example 2

### Adalimumab (Humira®) Treatment Efficacy in Patients with Psoriatic Arthritis Who Failed Prior DMARD Therapy

Patients with psoriatic arthritis (PsA) characteristically have increased concentrations of tumor necrosis factor (TNF) in their joints and skin lesions. TNF antagonist therapy has the potential to simultaneously improve the pathophysiology in both areas.

The objective of the following study was to evaluate the efficacy of adalimumab compared with placebo in patients with moderately to severely active psoriatic arthritis (PsA) who had an inadequate response to DMARD therapy. Patients with moderately to severely active PsA (≧3 swollen joints and ≧3 tender joints) who had an inadequate response to DMARD therapy were stratified by current use of DMARDs and randomized to receive either 40 mg adalimumab subcutaneously every other week (eow) or matching placebo for 12 weeks, followed by open-label (OL) therapy with adalimumab 40 mg eow. Results are reported for the blinded portion and the first 12 weeks of open label (OL) therapy.

Efficacy and safety data were collected from a Phase III, randomized, placebo-controlled, double-blind, multicenter study (Study X) and the first 12 weeks of an open-label extension study. FIG. 1 depicts the overview Study X design. Specifically, patients were stratified by DMARD use (yes/no), and then, randomized to receive either adalimumab 40 mg every other week (eow) or matching placebo for the first 12 weeks. Upon completion of 12 weeks of therapy, patients were eligible to enter the extension study, where adalimumab patients continued on 40 mg eow and placebo patients converted to adalimumab 40 mg eow.

Inclusion criteria for the study included: moderate to severely active PsA defined by ≧3 swollen joints and ≧3 tender or painful joints; ≧18 years old; inadequate response to DMARD therapy based on current or historic DMARD treatment; and presence of active cutaneous lesions of chronic plaque psoriasis or documented history of chronic plaque psoriasis. Exclusion criteria included: prior anti-TNF therapy; cyclosporine or tacrolimus within 4 weeks prior to Baseline; systemic psoriasis therapy within 4 weeks prior to Baseline; alefacept or siplizumab within 12 weeks prior to Baseline; other biologic or investigational therapy within 6 weeks prior to Baseline; and phototherapy or topicals within 2 weeks prior to Baseline. Efficacy measures included: ACR response criteria (primary endpoint: ACR20 response at Week 12); Disability Index of the Health Assessment Questionnaire (HAQ); Target Lesion evaluation and Physician's Global Assessment for psoriasis (in subjects with a psoriasis target lesion).

Table 1 shows that baseline demographics were similar between both groups except for a larger number of positive Rheumatoid Factor patients in the adalimumab arm.

TABLE 1
Baseline Demographics and Disease Characteristics

| | Placebo | Adalimumab |
|---|---|---|
| | eow | 40 mg eow |
| Characteristic | N = 49 | N = 51 |
| Age (yrs) | 47.7 ± 11.3 | 50.4 ± 11.0 |
| % Male | 51.0 | 56.9 |
| % Caucasian | 93.9 | 98.0 |
| Body Weight (kg) | 88.5 ± 21.1 | 91.5 ± 22.5 |
| Rheumatoid Factor negative (%) | 98.0 | 80.4** |
| Duration of PsA (yrs) | 7.2 ± 7.0 | 7.5 ± 7.0 |
| Psoriasis duration (yrs) | 13.8 ± 10.7 | 18.0 ± 13.2 |
| No. of previous DMARDs | 2.1 ± 1.3 | 1.7 ± 0.9 |

Mean ± SD except where specifically noted
**p ≦ 0.02, placebo vs adalimumab

Table 2 below shows that baseline disease characteristics were similar between both groups except for a higher CRP value in the placebo arm.

TABLE 2
Efficacy Measures at Baseline

| | Placebo | Adalimumab |
|---|---|---|
| | eow | 40 mg eow |
| Characteristic | N = 49 | N = 51 |
| Swollen Joint Count (0-76) | 18.4 ± 12.1 | 18.2 ± 10.9 |
| Tender Joint Count (0-78) | 29.3 ± 18.1 | 25.3 ± 18.3 |
| CRP (mg/L) | 1.6 ± 1.7 | 1.0 ± 1.0* |
| HAQ (0-3) | 1.0 ± 0.7 | 0.9 ± 0.5 |
| | n = 30 | n = 32 |
| Target Lesion Score (0-15)# | 8.1 ± 2.3 | 7.9 ± 1.8 |
| PGA (“Clear” or “Almost Clear”)# | 0 | 1 (3.1%) |

Mean ± SD except where specifically noted
**p ≦ 0.02, placebo vs adalimumab

Table 3 shows the disposition of patients as grouped in the double-blind or open-label groups.

TABLE 3
Disposition of Patients

| | Double-Blind | | Open-label |
|---|---|---|---|
| | Wk 0-12 | | Wk 12-24 |
| | Placebo | Adalimumab | Adalimumab |
| | Eow | 40 mg eow | 40 mg eow |
| | n (%) | n (%) | n (%) |
| Subjects entering study | 49 | 51 | 97 |
| Subjects completing study | 46 (94) | 50 (98) | 92 (95) |
| Subjects prematurely terminated | 3 (6) | 1 (2) | 5 (5) |
| Primary reason for termination: | | | |
| Adverse Event | 1 (2) | 1 (2)^{‡} | 3 (3) |
| Unsatisfactory therapeutic effect | 1 (2) | 0 | 0 |
| Other | 1 (2) | 0 | 2 (2) |

^{‡}Subject discontinued due to diverticulitis but was allowed to enter the open-label study.

ACR response rates were significantly better in the adalimumab group vs placebo at Week 12. Table 4 depicts the ACR response at weeks 12 and 24. At Week 24 (12 weeks of open-label therapy), after beginning adalimumab 40 mg eow, the placebo group experienced significant improvement in ACR scores compared with Week 12 values, while the adalimumab group continued to improve compared with Week 12. The slower response observed in the adalimumab-treated patients compared with the placebo/adalimumab-treated patients, and to patients in a previously reported study of adalimumab in PsA, may be related to their lower level of inflammation at baseline as reflected by the difference in baseline CRP. FIG. 2 also shows the ACR 20/50/70 response by week.

TABLE 4
ACR Response at Weeks 12 and 24

| % of Patients | | |
|---|---|---|
| ACR20 | ACR50 | ACR70 |

Week 12 (Blinded)

| | | | |
|---|---|---|---|
| Placebo (N = 49) | 16 | 2 | 0 |
| Adalimumab (N = 51) | 39* | 25*** | 14* |

Week 24 (Open-label)

| | | | |
|---|---|---|---|
| Placebo/Adalimumab (N = 49) | 57 | 37 | 22 |
| Adalimumab (N = 51) | 64 | 43 | 27 |

*p ≦ 0.005,
***p ≦ 0.001; placebo vs. adalimumab

Table 5 shows the mean change in HAQ at weeks 12 and 24. Mean improvement in HAQ scores were significantly better for patients receiving adalimumab compared with placebo at Week 12. At Week 24 (12 weeks of open-label therapy), after beginning adalimumab 40 mg eow, the placebo group significantly improved to a comparable level with the adalimumab group.

TABLE 5
Mean Change in HAQ at Weeks 12 and 24

Mean Change from Baseline

Week 12 (Blinded)

| | |
|---|---|
| Placebo | −0.1 |
| Adalimumab | −0.3** |

Week 24 (Open-label)

| | |
|---|---|
| Placebo/Adalimumab | −0.4 |
| Adalimumab | −0.3 |

Minimum Clinically Important Difference = −0.03; Mease PJ, et al. Ann Rheum Dis. 2004; 63(Suppl 1): 391-392.
**p ≦ 0.01, placebo vs. adalimumab.
Last observation carried forward.

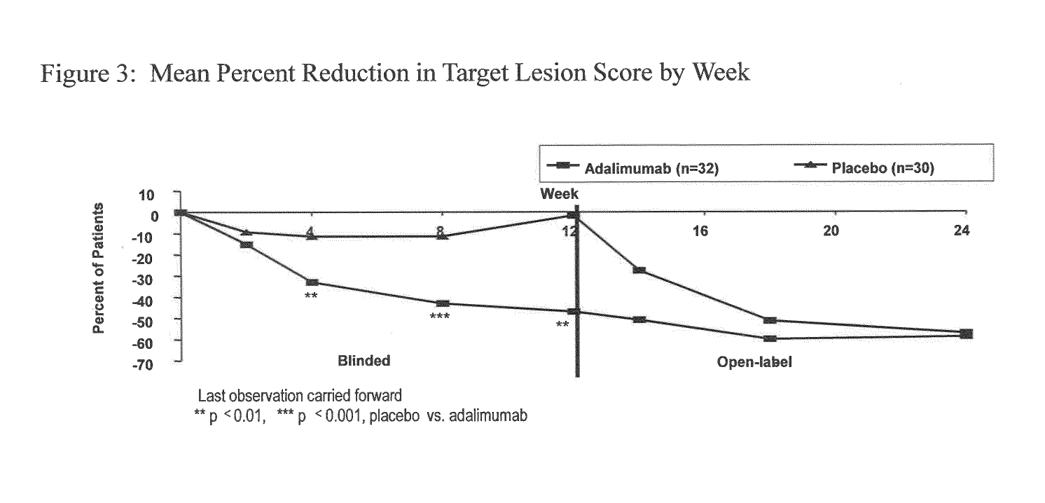

FIG. 3 shows the mean percent reduction in target lesion score by week. At Week 12, the mean percent reduction in Target Lesion score was significantly greater in the adalimumab group compared with placebo. At Week 24 (12 weeks of open-label therapy), after beginning adalimumab 40 mg eow, the placebo group rapidly improved while the adalimumab group continued to improve further.

Table 6 depicts the physician global assessment (clear or almost clear) at weeks 12 and 24. Physician Global Assessment rating of “Clear” or “Almost Clear” was significantly better for adalimumab patients compared with placebo patients at Week 12. At Week 24 (12 weeks open-label therapy), after beginning adalimumab 40 mg eow, the placebo group improved significantly while the adalimumab group continued to improve further.

TABLE 6
Physician Global Assessment (Clear or Almost Clear) at Weeks 12 and 24

% of Patients

Week 12 (Blinded)

| | |
|---|---|
| Placebo (N = 30) | 6.7 |
| Adalimumab (N = 32) | 40.6** |

Week 24 (Open-label)

| | |
|---|---|
| Placebo/Adalimumab (N = 30) | 46.7 |
| Adalimumab (N = 32) | 56.3 |

Last observation carried forward
**p ≦ 0.01, placebo vs. adalimumab

During the placebo-controlled portion of the study, the number (%) of patients with any AE was significantly lower for the adalimumab group. Table 7 shows the common adverse events ≧5% (blinded period). During the open-label period, 2 additional AEs were reported in ≧5% of all patients: cough (n=5, 5.2%), nasopharyngitis (n=5, 5.2%). No cases of TB, granulomatous infection, demyelination, drug-induced lupus, or CHF were observed. One patient was diagnosed with non-Hodgkins lymphoma after 1 dose of adalimumab. In retrospect, evidence of disease predated adalimumab treatment.

TABLE 7
Common Adverse Events ≧5% (Blinded Period).

| | Placebo | Adalimumab |
|---|---|---|
| | eow | 40 mg eow |
| | N = 49 | n = 51 |
| | n(%) | n(%) |
| Any AE | 39 (79.6) | 27 (52.9)** |
| Any SAE | 2 (4.1) | 1 (2.0) |
| Upper Respiratory Tract Infection NOS | 4 (8.2) | 7 (13.7) |
| Injection Site Pain | 6 (12.2) | 6 (11.8) |
| Ps aggravated | 8 (16.3) | 2 (3.9)* |
| Diarrhea NOS | 3 (6.1) | 1 (2.0) |
| Back Pain | 3 (6.1) | 1 (2.0) |
| PsA aggravated | 7 (14.3) | 1 (2.0)* |
| Headache | 3 (6.1) | 0 (0.0) |

*p ≦ 0.05,
**p ≦ 0.01, placebo vs. adalimumab for all comparisons.

The above study shows that adalimumab was effective in reducing the signs and symptoms of patients with moderate to severely active PsA who had failed DMARD therapy. Adalimumab was also safe and generally well-tolerated. Additional details regarding the above study can be found in Ann Rheum Dis 2005; 64(Suppl 111):313, which is incorporated by reference herein.

### Example 3

### Adalimumab Treatment Effects on Quality of Life in Patients with Psoriatic Arthritis: Results from Study G

Psoriatic arthritis (PsA) results in functional impairment in a large proportion of patients, including a progressive increase both in the number of affected joints and in the severity of joint damage, which can lead to discomfort, disfigurement, and disability. Effective treatment may significantly improve the quality of life in these patients.

The objective of this study was to evaluate the ability of adalimumab compared with placebo to improve quality of life in patients with moderate to severe PsA (Ann Rheum Dis 2005; 64(Suppl 111):317, incorporated by reference herein). To determine the ability of adalimumab to improve the quality of life in patients with moderate to severe PsA, adult patients with moderate to severely active PsA (≧3 swollen and ≧3 tender joints) who had an inadequate response to NSAIDs were included in the study. Patients were stratified for MTX use (yes/no) and extent of psoriasis (<3% or ≧3% Body Surface Area [BSA]), and randomized to receive either adalimumab 40 mg or placebo subcutaneously every other week for 24 weeks. Quality of life assessment instruments included the disability index of the Health Assessment Questionnaire (HAQ), the Short Form-36 Health Status Survey (SF-36) and the Fatigue scale of the Functional Assessment of Chronic Illness Therapy (FACIT-F). In patients with psoriasis involving ≧3% of BSA, Dermatology Life Quality Index (DLQI) was also assessed. Statistical comparisons were made for adalimumab treatment vs. placebo treatment.

The main inclusion criteria included: ≧18 years old; diagnosis of PsA; moderately to severely active disease as defined by ≧3 swollen joints and tender or painful joints; inadequate response or intolerance to NSAID therapy; and presence of active cutaneous lesions of chronic plaque psoriasis or documented history of chronic plaque psoriasis. Patients were stratified for methotrexate (MTX) use (yes/no) and extent of psoriasis (<3% or ≧3% Body Surface Area [BSA]), and randomized to receive either adalimumab 40 mg or placebo every other week (eow) for 24 weeks. Quality of life assessments were conducted at Weeks 12 and 24, including the following: Health Assessment Questionnaire disability index (HAQ); Short Form-36 Health Status Survey (SF-36); Fatigue Scale of the Functional Assessment of Chronic Illness Therapy (FACIT-Fatigue); and Dermatology Life Quality Index (DLQI; only in patients with 3% BSA psoriasis).

Patients were enrolled from 50 sites in North America and Europe with a total of 313 patients receiving treatment (N=151 adalimumab, N=162 placebo). Baseline characteristics were similar between treatment arms and consistent with moderate to severely active PsA. Table 8 below shows the baseline demographics and clinical characteristics.

TABLE 8
Baseline Demographics and Clinical Characteristics

| | Placebo | Adalimumab |
|---|---|---|
| | eow | 40 mg eow |
| | N = 162 | N = 151 |
| Age (years)‡ | 49.2 ± 11.1 | 48.6 ± 12.5 |
| Sex, Male | 54.9% | 56.3% |
| Race, Caucasian | 93.8% | 97.4% |
| Duration of psoriatic arthritis (yrs)‡ | 9.2 ± 8.7 | 9.8 ± 8.3 |
| Psoriasis duration (yrs)‡ | 17.1 ± 12.6 | 17.2 ± 12.0 |

‡Mean ± SD

Quality of life, as measured by SF-36, was similar at baseline for both groups. Table 9 below shows the QOL measures at baseline. Quality of life, as measured by SF-36, was similar at baseline for both groups (see Table 10 for baseline scores). Table 11 below shows the disposition of the patients (92% of patients completed the 24-week treatment period).

TABLE 9
QOL Measures at Baseline.

| | Placebo | Adalimumab |
|---|---|---|
| | eow | 40 mg eow |
| | N = 162 | N = 151 |
| HAQ (0-3) | 1.0 ± 0.7 | 1.0 ± 0.6 |
| FACIT-Fatigue (0-52)‡ | 30.8 ± 12.2 | 30.8 ± 12.1 |
| BSA psoriasis ≧3% | n = 70 | n = 70 |
| DLQI (0-30)‡ | 10.3 ± 7.5 | 8.6 ± 6.6 |

Mean ± SD for all values
‡n's may be slightly lower for some time points

TABLE 10
Baseline SF-36 Domain Scores.

| | Placebo | Adalimumab |
|---|---|---|
| | eow | 40 mg eow |
| | N = 162 | N = 151‡ |
| Physical Functioning | 48.2 | 50.8 |
| Role-Physical | 32.6 | 37.1 |
| Bodily Pain | 40.2 | 41.3 |
| General Health | 52.1 | 49.5 |
| Vitality | 41.6 | 41.4 |
| Social Functioning | 61.7 | 66.3 |
| Role-Emotional | 59.1 | 65.1 |
| Mental Health | 64.9 | 67.6 |
| Physical Component Score | 33.3 | 33.2 |
| Mental Component Score | 46.6 | 48.1 |

‡N is slightly lower for some responses.

TABLE 11
Disposition of Patients

| | Placebo | Adalimumab |
|---|---|---|
| | eow | 40 mg eow |
| | N = 162 | N = 151 |
| | n (%) | n (%) |
| Completed study | 149 (92.0) | 140 (92.7) |
| Subjects prematurely terminated | 13 (8.0) | 11 (7.3) |
| Primary reason for termination: | | |
| Adverse Event | 1 (0.6) | 3 (2.0) |
| Withdrew Consent | 5 (3.1) | 3 (2.0) |
| Abnormal laboratory value(s) | 0 | 2 (1.3) |
| Unsatisfactory therapeutic effect | 4 (2.5) | 1 (0.7) |
| Other | 3 (1.9) | 2 (1.3) |

The ACR20, 50, and 70 responses and the PASI 50, 75, and 90 responses for adalimumab-treated patients at Week 24 were significantly better than placebo. Disability improvement, as measured by the mean change in HAQ score, was both clinically (>0.3 units change) and statistically (p<0.001) significant in patients receiving adalimumab vs. placebo at Week 24. Table 12 depicts the ACR and PASI responses at week 24.

TABLE 12
ACR and PASI responses at Week 24

| | % of Patients | | |
|---|---|---|---|
| | ACR 20 | ACR 50 | ACR 70 |
| Placebo (N = 162) | 15 | 6 | 1 |
| Adalimumab (N = 151) | 57 | 39 | 23 |
| | PASI 50 | PASI 75 | PASI 90 |
| Placebo (N = 69) | 12 | 1 | 0 |
| Adalimumab (N = 69) | 75 | 59 | 42 |

All results, p ≦ 0.001 placebo vs. adalimumab.

Disability improvement, as measured by the mean change in HAQ score, was both clinically (>0.3 units change) and statistically (p<0.001) significant in patients receiving adalimumab vs. placebo at Week 24. Table 13 depicts the mean change in HAQ score at week 24. After 24 weeks of treatment, patients receiving adalimumab exhibited clinically significant changes (≧10 units change) in 7 of the 8 SF-36 domains while placebo patients did not demonstrate clinically significant changes in any domains. For 6 of these 7 domains with clinical significance, the changes were statistically significant compared with placebo. Table 14 depicts the mean change in SF-36 domains at week 24. Mean change from baseline in SF-36 Physical Component Summary score was statistically significant for adalimumab patients vs. placebo at Week 24 (see Table 15).

TABLE 13
Mean Change in HAQ Score at Week 24

| | Mean Change from Baseline |
|---|---|
| Placebo | −0.1 |
| Adalimumab | −0.4*** |

Minimum Clinically Important Difference = −0.03; Mease PJ, et al. Ann Rheum Dis. 2004; 63(Suppl 1): 391-392.
***p < 0.001, placebo vs. adalimumab.

TABLE 14
Mean Change in SF-36 Domains at Week 24

Mean Change from
Baseline

| | Placebo | Adalimumab |
|---|---|---|
| Physical Functioning | 2.9 | 15.8*** |
| Role-Physical | 8.9 | 30*** |
| Bodily Pain | 3.4 | 21.8*** |
| General Health | −0.1 | 11.6*** |
| Vitality | 1.7 | 12.8*** |
| Social Functioning | 2.6 | 11.8** |
| Role-Emotional | 4.6 | 10.3 |
| Mental Health | 1.1 | 4.5* |

***p < 0.001;
**p < 0.01;
*p < 0.05; placebo vs. adalimumab for all conditions
Minimum Clinically Important Difference (MCID) has been described in RA patients as a change of 5-10 units (Kosinski et al., Arthritis Rheum. 2000; 43: 1478-87). MCID has not been defined for PsA. Results described in the text are based on an MCID of 10 units.

TABLE 15
Mean Change in SF-36 Component Summary Scores at Week 24

Mean Change
from Baseline

| | Placebo | Adalimumab |
|---|---|---|
| Physical Component Summary | 1.4 | 9.3*** |
| Mental Component Summary | 1.2 | 1.6 |

***p < 0.001 placebo vs. adalimumab
Minimum Clinically Important Difference (MCID) has been described in RA as a change of 2.5-5.0 units for component summary scores (Kosinski et al., Arthritis Rheum. 2000; 43: 1478-87). MCID has not been defined for PsA.

At Weeks 12 and 24, statistically significant differences were seen in the mean change from baseline for FACIT-Fatigue scores of adalimumab- vs. placebo-treated patients. Table 16 depicts the mean change in FACIT-Fatigue score at weeks 12 and 24. In patients with ≧3% BSA psoriasis, mean changes from baseline in DLQI scores for the adalimumab group was clinically and statistically significant compared with placebo at Weeks 12 and 24. Table 17 shows the mean change in DLQI at weeks 12 and 24. As previously reported, adalimumab was generally well-tolerated during the study. (Mease P J, et al. Arthritis Rheum 2004; 50:4097).

TABLE 16
Mean Change in FACIT-Fatigue Score at Weeks 12 and 24

Mean Change
from Baseline

| | Placebo | Adalimumab |
|---|---|---|
| Week 12 | 0.6 | 6.5*** |
| Week 24 | 0.1 | 7.1*** |

Last observation carried forward.
The range of possible scores is 0-52, with a higher score reflecting reduction in overall fatigue.
***p < 0.001, placebo vs. adalimumab.
Minimum Clinically Important Difference (MCID) has been described as a change of 4 units (Cella D. et al., J. Pain Symptom Manage. 2002; 24: 547-61).

TABLE 17
Mean Change in DLQI at Weeks 12 and 24

Mean Change from Baseline

| | Placebo (N = 66) | Adalimumab (N = 66) |
|---|---|---|
| Week 12 | −0.4 | −5.6*** |
| Week 24 | −0.7 | −6.1*** |

Last observation carried forward.
The range of possible scores is 0-30 with a lower score reflecting improved QOL in relation to the patient's skin condition.
#n = 64 for placebo;
***p < 0.001, placebo vs. adalimumab
Minimum Clinically Important Difference (MCID) has been described for psoriasis patients as a change of 5 units (Khiji F. A. et al., Br. J. Dermatol. 2002; 147 Suppl. 62: 50).

The above study shows that adalimumab treatment significantly improves the quality of life in patients with moderate to severely active PsA. Mean changes in HAQ and DLQI demonstrated clinically and statistically greater improvement in adalimumab-vs. placebo-treated patients. Clinically and statistically significant changes were also seen in most SF-36 domains for patients on adalimumab vs. placebo. FACIT-Fatigue scores were significantly (p<0.001) better for adalimumab-treated patients compared with placebo.

### Example 4

### Improvement in Health Utility in Patients with Psoriatic Arthritis Treated with Adalimumab (HUMIRA®)

Cost utility analyses (CUA), which combine both quality and length of life into a single measured called Quality-Adjusted Life Years (QALYs), have been mandated as the preferred measure of effectiveness in economic evaluations.

The objective of this study was to estimate change in health state utilities in patients with psoriatic arthritis (PsA) receiving adalimumab vs. placebo, as measured by the health utility measurement Short Form 6D (SF-6D). To further this objective, in a placebo-controlled, Phase III trial of adalimumab (Study G), patients with active PsA (n=313) randomly received adalimumab 40 mg every other week (eow) or placebo for 24 weeks after consideration of disease duration and prior methotrexate use (Ann Rheum Dis 2005; 64(Suppl III):401). A subgroup of patients were assessed for improvement in psoriatic lesions based on affected body surface area (BSA) of ≧3% at baseline (n=140, 46%). The SF-6D was estimated at baseline, 12 weeks and 24 weeks using the Brazier algorithm of responses to the Short Form 36 (SF-36) patient questionnaire. Responses were used to derive the SF-6D, a preference-based utility instrument. Multiple linear regression models were built to explore the effects of age, sex, disease duration, concomitant therapies, baseline Health Assessment Questionnaire Disability Index (HAQ DI), and the Psoriasis Area and Severity Index (PASI). Percentage change in utility from baseline at 24 weeks was the primary outcome. Patients were further differentiated as responders or non-responders using the Psoriatic Arthritis Response Criteria (PsARC), improvement in American College of Rheumatology criteria by 50% (ACR50), and an improvement in the Psoriasis Area and Severity Index (PASI) score by 75% (PASI 75). Determinants of the change in utility score were analyzed using regression analysis estimated by Generalized Estimating Equations (GEE) approach (with an exchangeable correlation matrix). Table 18 shows the baseline demographic and clinical characteristics.

TABLE 18
Baseline Demographics and Clinical Characteristics.

| | Placebo | Adalimumab |
|---|---|---|
| | Mean (SD) or % | Mean (SD) or % |
| N | 162 | 151 |
| Age (yrs) | 49 (11.1) | 49 (12.5) |
| Male | 55% | 56% |
| Duration of PsA symptoms (yrs) | 9.2 (8.7) | 9.8 (8.3) |
| Duration of psoriasis (yrs) | 17 (12.6) | 17 (12.0) |
| Number of previous DMARDs | 1.5 (1.2) | 1.5 (1.1) |
| Concomitant Methotrexate | 55% | 52% |
| Concimitant Corticosteroids | 14% | 15% |
| Number of tender joints | 26 (18.0) | 24 (17.3) |
| Number of swollen joints | 14 (11.1) | 14 (12.2) |
| HAQ | 1 (0.67) | 1 (0.62) |
| SF-6D | 0.65 (0.09) | 0.66 (0.09) |
| % with non-zero PASI score | 43% | 46% |
| PASI | 8.3 (7.3) | 7.4 (6.1) |

SD = standard deviation

Mean baseline patient characteristics included age=49 years, disease duration=9.5 years, tender joint count=25, swollen joint count=14, HAQ DI=1.0, and PASI=7.9. Baseline SF-6D values were 0.66 and 0.65 for the adalimumab and placebo arms respectively. For patients with skin involvement, the baseline SF-6D values were 0.68 for adalimumab vs. 0.65 for placebo.

For patients without skin involvement, the baseline SF-6D values were and 0.65 vs. 0.65 respectively. Overall, adalimumab improved health utility vs. placebo by a factor of 3 (p<0.01). Table 19 shows that adalimumab was particularly efficacious in patients with skin involvement (≧3% body surface area [BSA]). PsARC response was a significant predictor of utility improvement, and, for patients with skin involvement, PASI 75 was also important. Table 19 shows the percentage of improvement in health utilities over 6 months.

TABLE 19
Percentage Improvements in Health Utilities Over 6 Months.

| | Adalimumab | Placebo |
|---|---|---|
| | (N = 151) % (SD) | (N = 162) % (SD) |
| Overall | 10.6 (18.9) | 2.9 (16.2) |
| PsARC responders | 15.1 (20.0) | 7.5 (16.5) |
| PsARC non-responders | 0.8 (13.2) | 0.9 (15.9) |
| Baseline BSA <3% | 8.3 (17.1) | 4.3 (15.7) |
| PsARC responders | 12.9 (17.2) | 9.7 (14.9) |
| PsARC non-responders | −4.3 (10.3) | 1.7 (15.5) |
| Baseline BSA ≧3% | 13.7 (20.9) | 0.5 (17.0) |
| PsARC responders | 18.4 (23.5) | 2.9 (19.6) |
| PsARC non-responders | 5.9 (14.0) | −0.4 (16.7) |
| PASI75 responders | 17.3 (23.0) | NA |
| PASI75 non-responders | 5.8 (14.1) | 1.0 (17.3) |

SD = standard deviation.

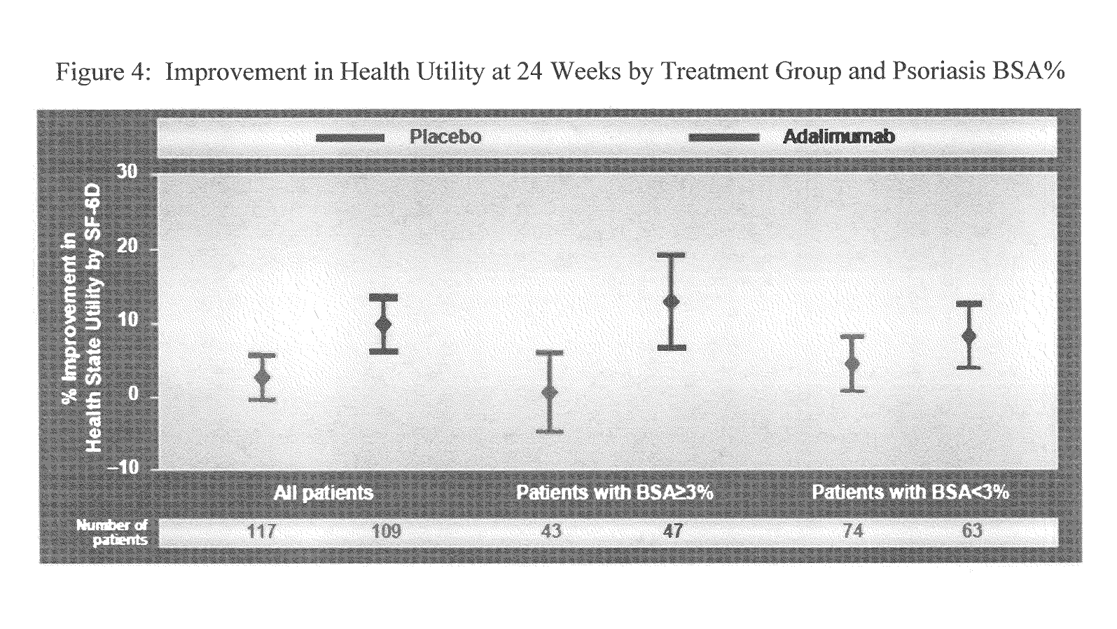

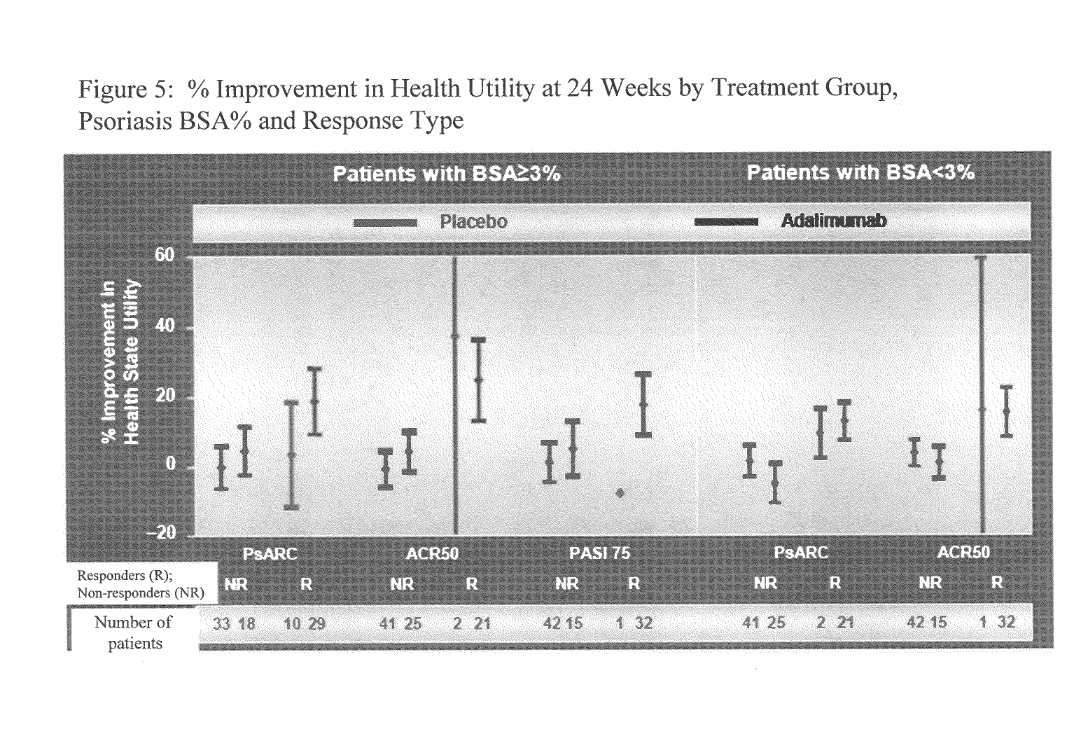

Overall, a statistically significantly greater improvement in health utility was seen in patients on adalimumab (10%) vs. placebo (3%) (p<0.01) (See FIG. 4). Patients with skin and joint disease (BSA≧3%) showed greatest improvement in health state utility with adalimumab vs. placebo Improvement in health utility, as measured by SF-6D, was found to be closely linked to the type of response attained (whether measured by PsARC, ACR50, or PASI 75) (See FIG. 5). Since the response rates were higher in patients treated with adalimumab vs. placebo, overall utility improvement was higher (See Table 20). Response in skin symptoms (PASI 75) appears at least as important as response to joint symptoms. This was confirmed in a multivariate analysis, where PASI is an independent statistically significant predictor of health utility among patients with skin disease (BSA≧3%) (p<0.0001). Table 21 shows the relationship between SF-6D, HAQ and PASI.

TABLE 20
Response Rates at 24 Weeks by Treatment
Group and Psoriasis BSA %.

| | Placebo | | Adalimumab | |
|---|---|---|---|---|
| | BSA <3% | BSA ≧3% | BSA <3% | BSA ≧3% |
| N | 92 | 70 | 81 | 70 |
| Mean Baseline | 0.65 (0.09) | 0.65 (0.10) | 0.68 (0.08) | 0.65 (0.10) |
| SF-6D (SD) | | | | |
| 24 Weeks | | | | |
| Mean SF-6D | 0.67 (0.10) | 0.66 (0.10) | 0.72 (0.09) | 0.71 (0.11) |
| (SD) | | | | |
| PSARC (%) | 26 | 20 | 64 | 56 |
| ACR20 (%) | 16 | 14 | 60 | 53 |
| ACR50 (%) | 5 | 6 | 42 | 36 |
| ACR70 (%) | 2 | 0 | 22 | 23 |
| PASI 50 (%) | | 12 | | 75 |
| PASI 75 (%) | | 1 | | 59 |
| PASI 90 (%) | | 0 | | 42 |

SD = standard deviation

TABLE 21
Estimated Relationship Between SF-6D^{†}, HAQ and PASI*

| | Coefficient | SE | p-value |
|---|---|---|---|
| BSA <3% | | | |
| Intercept | 0.69 | 0.031 | <0.0001 |
| HAQ | −0.54 | 0.035 | <0.0001 |
| BSA ≧3% | | | |
| Intercept | 0.83 | 0.052 | <0.0001 |
| HAQ | −0.56 | 0.045 | <0.0001 |
| PASI* | −0.10 | 0.022 | <0.0001 |

SF-6D† is transformed by: SF-6D transformed = log (Y/(1 − Y)) where Y = 0.95(SF-6D − 0.263)/(1 − 0.263) + 0.025.
PASI* is transformed by: PASI transformed = ln (PASI + 0.5).
SE = standard error.

An important outcome of any clinical intervention is the change in the patient's perceived state of health. These findings demonstrate that adalimumab was efficacious in improving PsA patients' health state utilities; this efficacy was observed to an even higher degree in patients with more skin involvement. Health utilities, when modeled with disease-related costs over patients' lifetimes, can help demonstrate the cost effectiveness (cost/QALY) of adalimumab.

### Example 5

### Adalimumab Treatment with and without Methotrexate in Patients with Moderate to Severe Psoriatic Arthritis: Results from Study G

Tumor necrosis factor (TNF) concentrations are elevated in the skin and joints of patients with psoriatic arthritis, an inflammatory arthropathy. Adalimumab, a fully human monoclonal antibody, binds to TNF and inhibits the inflammatory response. In rheumatoid arthritis (RA), adalimumab may be used in combination with methotrexate (MTX) or as monotherapy. Studies in RA have demonstrated a synergistic effect for TNF antagonists when used in combination with MTX, but in PsA, this has not been shown.

The objective of this study was to compare the efficacy of adalimumab administered with and without MTX in patients with moderate to severe PsA (Ann Rheum Dis 2005; 64(Suppl III):325). In order to determine the efficacy of adalimumab administered with and without MTX in patients with moderate to severe PsA, adult patients with moderately to severely active PsA (≧3 swollen joints and ≧3 tender joints) who had an inadequate response to NSAID therapy were included in the study. Patients were stratified for MTX use (yes/no) and degree of psoriasis (<3% or ≧3% Body Surface Area [BSA]), and then, randomized to receive either adalimumab 40 mg or placebo subcutaneously every other week for 24 weeks. In this post-hoc analysis, the effect of adalimumab alone vs. adalimumab plus MTX was evaluated using ACR response criteria in all patients and the Psoriasis Area and Severity Index (PASI) response criteria in patients who had ≧3% BSA psoriasis involvement at baseline (see Table 22).

TABLE 22
Efficacy Results at Week 24

| | Adalimumab + MTX | Adalimumab Only |
|---|---|---|
| | N = 77 | N = 74 |
| ACR20/50/70 (%) | 55/36/22 | 59/42/23 |
| HAQ, mean change | −0.4 | −0.4 |
| | n = 29 | n = 40 |
| PASI 50/75/90 (%) | 86/72/52 | 70/53/35 |

This was a Phase III, randomized, parallel-group, placebo-controlled, double-blind trial, conducted in the US, the UK, Canada, France, Germany, Belgium, Italy, and Austria. Patients were randomized in a 1-to-1 fashion to receive placebo or adalimumab 40 mg every other week (eow), administered subcutaneously. Randomization was centrally stratified by MTX use and extent of psoriasis (<3% or ≧3% body surface area [BSA] involvement at baseline). Inclusion criteria included: moderate to severely active PsA defined by ≧3 swollen joints and ≧3 tender or painful joints; inadequate response or intolerance to NSAID therapy; history of psoriasis; and age≧18 years. Exclusion criteria included: prior anti-TNF therapy; alefacept within 12 weeks prior to study entry; other biologics within 6 weeks prior to study entry; DMARDs (except MTX) within 4 weeks prior to study entry; systemic therapies for psoriasis within 4 weeks prior to study entry; and phototherapy and topicals within 2 weeks prior to study entry. Enrollment screening included chest x-ray, electrocardiogram, PPD skin test, and routine labs. Patients were allowed to receive rescue therapy with steroids or DMARDs following the Week 12 evaluation, if they failed to have a 20% decrease in both swollen and tender joint counts for 2 consecutive visits. Study visits were conducted at Weeks 2, 4, and then every 4 weeks until Week 24. Efficacy measures included: ACR response criteria (co-primary endpoint: ACR20 response at Week 12); Health Assessment Questionnaire disability index (HAQ); and Psoriasis Area and Severity Index (PASI) in patients with significant psoriasis at study entry (≧3% BSA). All patients completing the 24 weeks were eligible for long-term treatment in an open-label extension study.

A total of 151 patients received adalimumab and approximately 50% were on concomitant MTX. Overall, both monotherapy and combination therapy groups were similar in baseline demographics. For baseline disease characteristics, patients receiving adalimumab with concomitant MTX had a shorter duration of PsA and a lower SJC compared with those receiving adalimumab alone. Baseline demographics and disease characteristics are shown in Table 23 below.

TABLE 23
Baseline Demographics and Disease Characteristics.

| | Adalimumab | Adalimumab + |
|---|---|---|
| | Alone | MTX |
| Characteristic‡ | N = 74 | N = 77 |
| Age (yrs) | 49.3 ± 13.0 | 48.0 ± 12.1 |
| Sex, Male | 58.1% | 54.5% |
| Race, Caucasian | 95.9% | 98.7% |
| Duration of psoriatic arthritis | 11.4 ± 9.0 | 8.3 ± 7.3* |
| (yrs) | | |
| Psoriasis duration (yrs) | 18.5 ± 11.9 | 16.0 ± 12.1 |
| MTX dose (mg) | NA | 17.3 ± 5.2 |
| Previous DMARDs | 1.2 ± 1.2 | 1.8 ± 1.1 |

‡ Mean ± SD except where specifically noted
*p < 0.05, adalimumab + MTX vs adalimumab alone
NA = not applicable

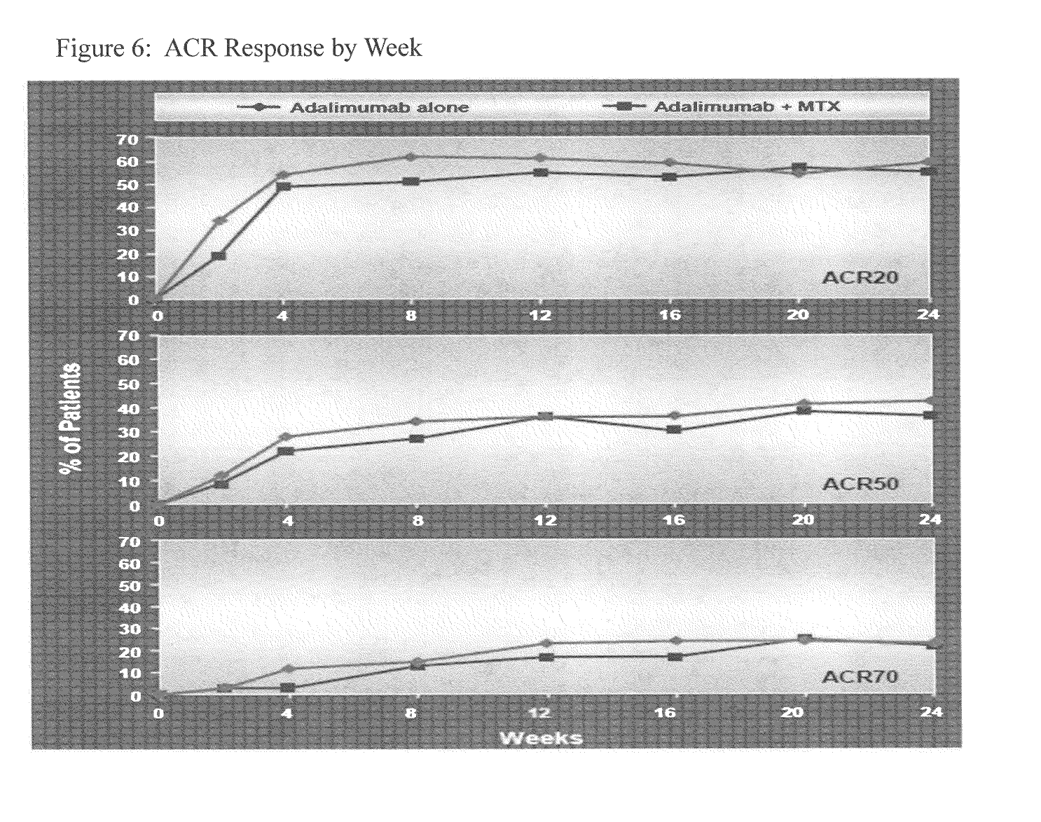

For the adalimumab alone group, 51 (68.9%) had been previously treated with a DMARD and 41 (55.4%) had received MTX. Efficacy measures at baseline are shown in Table 24. Patients receiving adalimumab alone showed similar improvements in ACR responses compared with those receiving adalimumab with MTX at Weeks 12 and 24. Table 25 depicts ACR response at weeks 12 and 24. Rapid and sustained improvement in ACR20 response for patients receiving adalimumab alone and in those with concomitant MTX. ACR response by week is shown in FIG. 6.

TABLE 24
Efficacy Measures at Baseline.

| | Adalimumab | Adalimumab + |
|---|---|---|
| | Alone | MTX |
| Characteristic | N = 74 | N = 77 |
| CRP (mg/L) | 1.2 ± 2.0 | 1.5 ± 2.2 |
| Swollen Joint Count (0-76) | 16.3 ± 14.1 | 12.4 ± 9.7* |
| Tender Joint Count (0-78) | 25.7 ± 17.8 | 22.2 ± 16.8 |
| HAQ (0-3) | 1.0 ± 0.6 | 1.0 ± 0.6 |
| MTX dose (mg) | NA | 17.3 ± 5.2 |
| BSA psoriasis ≧3% | n = 40 | n = 29 |
| PASI (0-72) | 7.1 ± 5.2 | 7.9 ± 7.2 |
| DLQI (0-30)^{#} | 9.8 ± 6.8 | 6.9 ± 6.0^{†} |

Mean ± SD except where specifically noted
*p < 0.05,
^{†}p ≦ 0.1, adalimumab + MTX vs adalimumab alone
^{#}adalimumab, n = 39;
Adalimumab + MTX, n = 27

TABLE 25
ACR Response at Weeks 12 and 24

| % of Patients | | |
|---|---|---|
| ACR20 | ACR50 | ACR70 |

Week 12

| | | | |
|---|---|---|---|
| Adalimumab alone (N = 74) | 61 | 36 | 23 |
| Adalimumab + MTX (N = 77) | 55 | 36 | 17 |

Week 24

| | | | |
|---|---|---|---|
| Adalimumab alone (N = 74) | 59 | 42 | 23 |
| Adalimumab + MTX (N = 77) | 55 | 36 | 22 |

p = NS for all comparisons.

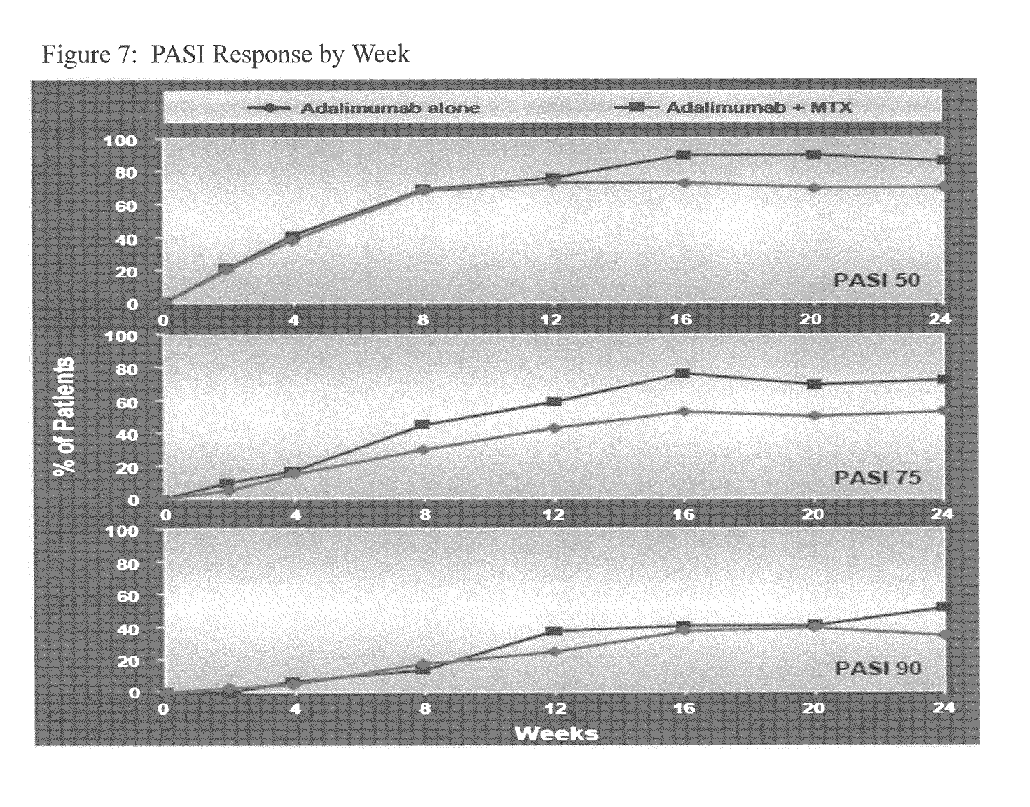

Both groups demonstrated clinically significant improvement in mean HAQ response. Table 26 depicts the HAQ response at weeks 12 and 24. As shown in Table 27, the PASI response rates observed when adalimumab was administered with MTX were higher than those seen with monotherapy, however, these differences were not statistically significant. Rapid and sustained improvement in PASI response for patients receiving adalimumab alone and in those with concomitant MTX. FIG. 7 depicts the PASI response by week.

TABLE 26
HAQ Response at Weeks 12 and 24

Mean Change
From Baseline

| | Adalimumab Alone | Adalimumab + MTX |
|---|---|---|
| Week 12 | −0.4 | −0.3 |
| Week 24 | −0.4 | −0.4 |

Minimum Clinically Important Difference = −0.3; Mease PJ, et al. Ann Rheum Dis. 2004; 63(Suppl 1): 391-392.

TABLE 27
PASI Response at Weeks 12 and 24

| % of Patients | | |
|---|---|---|
| PASI 50 | PASI 75 | PASI 90 |

Week 12

| | | | |
|---|---|---|---|
| Adalimumab alone (N = 40) | 73 | 43 | 25 |
| Adalimumab + MTX (N = 29) | 76 | 59 | 38 |

Week 24

| | | | |
|---|---|---|---|
| Adalimumab alone (N = 40) | 73 | 43 | 25 |
| Adalimumab + MTX (N = 29) | 76 | 59 | 38 |

p = NS for all comparisons.

Adalimumab was generally safe and well-tolerated during this study. Table 28 below shows the treatment emergent adverse events ≧5%. Elevations of transaminases were more common on lab evaluations in adalimumab-treated patients. The majority of patients were on concomitant hepatotoxins (mainly MTX) and had elevations that were transient, returning to normal while on study drug. The rate of infectious AEs was not clinically different between the 2 groups. No events of tuberculosis, granulomatous infection, malignancy, demyelination, drug-induced lupus, or CHF were observed.

TABLE 28
Treatment Emergent Adverse Events ≧5%.

| | Adalimumab | Adalimumab + |
|---|---|---|
| | Alone | MTX |
| | N = 74 | N = 77 |
| | n (%) | n (%) |
| Any AE | 60 (81.1%) | 62 (80.5%) |
| Any SAE | 2 (2.7%) | 3 (3.9%) |
| Influenza-like illness | 1 (1.4%) | 4 (5.2%) |
| Injection site reaction NOS | 2 (2.7%) | 8 (10.4%) |
| Sinusitis NOS | 3 (3.7%) | 1 (1.3%) |
| ALT increased | 2 (2.7%) | 4 (5.2%) |
| LFT NOS abnormal | 3 (4.1%) | 4 (5.2%) |
| Hypertension NOS | 1 (1.4%) | 7 (9.1%) |

Overall, adalimumab was effective in treating the signs and symptoms of psoriatic arthritis as monotherapy or in combination with MTX. Furthermore, adalimumab was generally safe and well-tolerated. These results are consistent with previous reports of other TNF antagonists in PsA. Limitations: This post-hoc analysis compared the efficacy of adalimumab therapy added to stable MTX patients versus adalimumab alone. A study of PsA patients receiving TNF therapy compared with MTX therapy in MTX naïve patients may be indicated.

### Example 6

### Adalimumab Inhibits Radiographic Disease Progression in Patients with Psoriatic Arthritis

Traditional, non-biologic DMARDs have not been shown to effectively inhibit the radiographic progression of joint damage in PsA. Erosive polyarthritis occurs in a substantial proportion of patients with psoriatic arthritis (PsA). Adalimumab, a fully human anti-tumor necrosis factor monoclonal antibody, has been shown to inhibit radiographic progression when used to treat patients with moderate to severe rheumatoid arthritis. The objective of this study was to determine whether adalimumab can effectively inhibit the radiographic progression of joint disease in patients with moderate to severe PsA.

This was a Phase III, randomized, double-blind, placebo-controlled study of adult patients with moderate to severely active PsA. Patients were stratified by methotrexate (MTX) use (yes/no) and degree of psoriasis (<3% or ≧3% body surface area). Patients were randomized in a 1:1 fashion to receive either adalimumab 40 mg or matching placebo every other week (eow) for 24 weeks. The inclusion criteria included patients with: ≧3 swollen and ≧3 tender joints; inadequate response to NSAID therapy; a history of psoriasis; age≧18 years. The exclusion criteria included: prior anti-TNF therapy; Alefacept within 12 weeks prior to study entry; other biologics within 6 weeks prior to study entry; DMARDs (except MTX) within 4 weeks prior to study entry; systemic therapies for psoriasis within 4 weeks prior to study entry; phototherapy and topicals within 2 weeks prior to study entry. See table 29 for baseline demographic data.

TABLE 29
Baseline Demographics and Clinical Characteristics

| | Placebo eow | Adalimumab 40 mg eow |
|---|---|---|
| | N = 162 | N = 151 |
| Age (yrs) | 49.2 ± 11.1 | 48.6 ± 12.5 |
| Sex, Male | 54.9% | 56.3% |
| Race, Caucasian | 93.8% | 97.4% |
| Duration of psoriatic arthritis | 9.2 ± 8.7 | 9.8 ± 8.3 |
| (yrs) | | |
| Psoriasis duration (yrs) | 17.1 ± 12.6 | 17.2 ± 12.0 |
| Swollen Joint Count (0-76) | 14.3 ± 11.1 | 14.3 ± 12.2 |
| Tender Joint Count (0-78) | 25.8 ± 18.0 | 23.9 ± 17.3 |
| HAQ (0-3) | 1.0 ± 0.7 | 1.0 ± 0.6 |
| mTSS | 19.1 ± 35.5 | 22.7 ± 46.0 |
| ERO | 10.0 ± 19.7 | 11.4 ± 25.5 |
| JSN | 9.2 ± 16.9 | 11.2 ± 21.9 |
| Methotrexate use | 50% | 51% |

Mean ± SD except where specifically noted

Patients who completed the 24-week, double-blind study were eligible to enroll in an open-label extension (OLE) study in which all patients received adalimumab 40 mg eow. See table 30 for the disposition of the patients in the study.

TABLE 30
Disposition of Patients

| | Double-Blind | | Open-Label |
|---|---|---|---|
| | Wk 0-24 | | Wk 24-48 |
| | | Adalimumab | Adalimumab |
| | Placebo | 40 mg eow | 40 mg eow |
| | Eow n (%) | n (%) | n (%) |
| Adalimumab | 162 | 151 | 285 |
| 40 mg eow | | | |
| n (%) | | | |
| Subjects completing study* | 149 (92) | 140 (93) | 272 (95) |
| Subjects prematurely terminated | 13 (8) | 11 (7) | 13 (5) |
| Primary reason for termination: | | | |
| Adverse Event | 1 (1) | 3 (2) | 2 (1) |
| Withdrew consent | 5 (3) | 3 (2) | 3 (1) |
| Abnormal laboratory value(s) | 0 | 2 (1) | 0 |
| Unsatisfactory therapeutic effect | 4 (3) | 1 (1) | 3 (1) |
| Other | 3 (2) | 2 (1) | 5 (2) |

After 12 weeks of treatment with open label therapy, patients failing to meet pre-specified criteria were eligible to receive 40 mg weekly. Radiographic assessments were performed during both the blinded portion (Weeks 0 and 24) and the OLE (Week 48). Radiographs of the hands and feet were assessed by a modified Total Sharp Score (mTSS) in which additional joints typically involved in PsA were added and the numerical scales expanded.

Clinical findings associated with PsA (eg, pencil-in-cup changes) were also evaluated. Inclusion in the Week 24 analysis required both Baseline and Week 24 films where at least 50% of the joints were evaluable. Various sensitivity analyses were used to account for missing patient films: imputation of zero change from baseline, imputation of the worst rank, imputation of the 50th/75th percentile change based on patients with similar baseline scores. Week 48 analysis included all patients from the Week 24 analysis. If a Week 48 film was not available (or <50% of the joints evaluable), then the following imputation was performed: if originally randomized to placebo, change of 0 was imputed; if originally randomized to adalimumab, linear extrapolation using first two films was conducted. All films were read by two independent readers who were blinded to treatment and film order. Read #1 was an evaluation of baseline and Week 24 films and Read #2 was an evaluation of baseline, Week 24, and Week 48 films.

Baseline demographics and disease severity characteristics were consistent with moderate to severe PsA and were well-matched between treatment arms (See table 29). Out of evaluable films at week 24, the number of placebo subjects was 152, and the number adalimumab subjects was 144. Out of evaluable films at week 48, the number of placebo/adalimumab subjects was 134, and the number of adalimumab subjects was 128. As a radiographic scoring method, the Modified Total Sharp Score (mTSS) was determined according to the following criteria: joint space narrowing was assessed at 48 sites, each site receiving a score between 0-4, and erosion was assessed at 54 sites, each site receiving a score between 0-7. The range of possible scores for joint space narrowing was consequently 0-192, and the range of possible scores for erosion was 0-378. The sum of these values determined the mTSS, which could range from 0-570. Other radiographic findings associated with PsA include phalangeal tuft resorption (measurable at 12 sites), subluxation (26 sites), pencil-in-cup (18 sites), periostitis (38 sites), and juxta-articular periostitis (52 sites). As previously reported, the ACR20, 50, and 70 responses and the PASI 50, 75, and 90 responses for adalimumab-treated patients at Week 24 were significantly better than placebo (see Table 31).

TABLE 31
ACR and PASI Responses at Week 24

| | % of Patients | | |
|---|---|---|---|
| | ACR 20 | ACR 50 | ACR 70 |
| Placebo (N = 162) | 15 | 6 | 1 |
| Adalimumab (N = 151) | 57 | 39 | 23 |
| | PASI 50 | PASI 75 | PASI 90 |
| Placebo (N = 69) | 12 | 1 | 0 |
| Adalimumab (N = 69) | 75 | 59 | 42 |

All results, p ≦ 0.001 placebo vs. adalimumab.

The distribution of mTSS scores demonstrated that fewer patients treated with adalimumab had an increase in structural damage during 24 weeks of treatment compared with placebo. The mean change in mTSS at Week 24 was 1.0 and −0.2 for the placebo and adalimumab treatment groups, respectively (p≦0.001 using ranked ANOVA). The number and percent of patients who had an increase in Sharp score during the study are shown in Table 32.

Statistically significant differences were observed between adalimumab and placebo treated subjects for both erosion scores and joint space narrowing scores (p≦0.001 using a ranked ANCOVA). Erosion is the change from baseline of 0.0 in adalimumab vs. 0.6 in placebo. Joint Space Narrowing is the change from baseline of −0.2 in adalimumab vs. 0.4 in placebo.

Sensitivity analyses to account for missing patient films were performed and results maintained statistical significance with all analyses. Post-hoc sensitivity analyses excluding (1) feet and (2) DIPs demonstrated that statistical significance was maintained in both analyses.

Approximately 3 times as many placebo-treated patients had an increase in mTSS (>0.5 units) than adalimumab-treated patients during the first 24 weeks of treatment (Table 32).

TABLE 32
Change* in Modified Total Sharp Score at Week 24

| | Placebo | Adalimumab |
|---|---|---|
| | N = 152 n (%) | N = 144 n (%) |
| Decrease in Sharp Score | 8 (5.3%) | 27 (18.8%) |
| No change in Sharp Score | 100 (65.8%) | 104 (72.2%) |
| Increase in Sharp Score | 44 (28.9%) | 13 (9.0%) |

p ≦ 0.001 placebo vs. adalimumab using CMH test
*Change defined as >0.5 units in mTSS Score

Table 33 shows that statistically significant differences were observed between adalimumab- and placebo-treated subjects, regardless of whether concomitant MTX was being used. Mean differences were slightly higher for the patients taking concomitant MTX.

TABLE 33
mTSS of Subjects With and Without MTX

| | N | Baseline | Mean Change |
|---|---|---|---|
| With MTX | | | |
| Placebo | 78 | 25.0 | 1.2 |
| Adalimumab | 76 | 21.7 | −0.3*** |
| Without MTX | | | |
| Placebo | 74 | 14.6 | 0.9 |
| Adalimumab | 68 | 22.9 | −0.1*** |

***p ≦ 0.001 vs. placebo for ranked ANCOVA.

The prevalence of PsA-associated findings is shown in Table 34. No significant difference was found between groups at baseline and no significant progression was found in either group during the 24-week study.

TABLE 34
Change Prevalence of PsA-Associated Findings

| | All Patients |
|---|---|
| | (N = 313) |
| | n (%) |
| Joint space widening | 38 (12.1%) |
| Gross osteolysis | 60 (19.2%) |
| Subluxation | 49 (15.7%) |
| Pencil-in-cup | 9 (2.9%) |
| Juxta-articular periostitis | 247 (78.9%) |
| Shaft periostitis | 140 (44.7%) |
| Phalangeal tuft resorption | 224 (71.6%) |

Table 35 shows a lack of changes in mTSS observed with adalimumab treatment during the first 24 weeks that was maintained at Week 48. Patients treated with placebo for 24 weeks did not have radiographic progression of disease during the open-label period.

TABLE 35
Mean Change in mTSS at Week 48

| | | | 24 Wk Mean | 48 Wk Mean |
|---|---|---|---|---|
| | N | Baseline | Change | Change |
| Placebo | 152 | 21.8 | 0.9 | 1.0 |
| Adalimumab | 144 | 23.7 | −0.1*** | 0.1 |

***p ≦ 0.001 adalimumab vs. placebo

In conclusion, adalimumab was more effective compared with placebo in inhibiting radiographic disease progression over a 24-week period. Adalimumab showed differences versus placebo both in patients taking concomitant methotrexate and in those taking adalimumab as monotherapy. The inhibition of structural damage progression observed in adalimumab-treated patients at 24 weeks was maintained at one year.

### Example 7

### A Comparative Cost-Consequence Analysis of TNF Antagonists in the Treatment of Psoriatic Arthritis

Preference-based utilities are an ideal measure of therapeutic effectiveness in multifaceted diseases such as psoriatic arthritis (PsA). A study estimating health utility improvements in PsA patients was performed in order to help determine the costs and consequences attributable to treatment with the TNF antagonists adalimumab, etanercept and infliximab.

A health utility endpoint, the SF-6D (a community-based preference measure suitable for economic evaluations), was used to estimate the cost and efficacy of each TNF antagonist. The SF-6D was estimated at baseline and at 24 weeks using the Brazier algorithm to transform SF-36 responses from an adalimumab trial in PsA (Study G). To obtain comparable SF-6D utility scores from trials of etanercept and infliximab the following algorithm was adapted. First, covariate-adjusted improvements in HAQ and PASI were calculated for each trial using reported ACR and PASI response rates. Covariates considered included age, gender, disease duration, concomitant therapies, and baseline HAQ and PASI. Second, regression coefficients obtained by modeling the relationship between HAQ and PASI with the SF-6D from results of Study G were used to estimate the SF-6D scores for patients in the etanercept and infliximab trials. Costs of drug acquisition, administration and monitoring were estimated for standard doses of each drug. The resulting estimates of cost (US Dollars) and efficacy of each TNF antagonist are summarized in Table 36.

TABLE 36
Percentage Improvement in Health Utility at 24 Weeks by Treatment Group and Psoriasis Patient BSA %

| | Etanercept | | Adalimumab | | Infliximab | | MTX | |
|---|---|---|---|---|---|---|---|---|
| | 25 mg twice weekly | | 40 mg eow | | 5 mg/kg | | 15 mg/wk | |
| | N = 101 | | N = 151 | | N = 100 | | N = 81 | |
| | BSA > 3% | BSA ≦ 3% | BSA > 3% | BSA ≦ 3% | BSA > 3% | BSA ≦ 3% | BSA > 3% | BSA ≦ 3% |
| Percentage Improvement | 6.1% | 6.1% | 8.5% | 5.8% | 9.0% | 5.8% | 1.0% | 1.0% |
| in Health State Utility | | | | | | | | |

| | | | | |
|---|---|---|---|---|
| Drug Cost (WAC) $ | 7,538 | 7,538 | 11,560 | 121 |
| Monitoring/Administration $ | 473 | 473 | 1,080 | 746 |

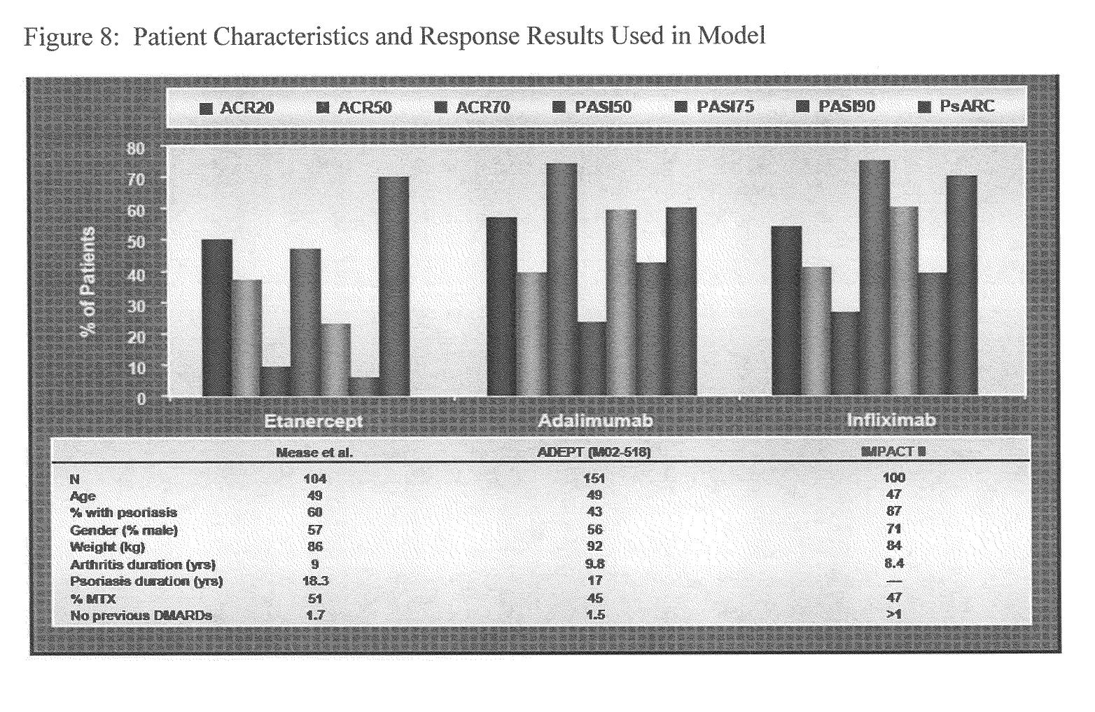

Efficacy was based on a review of published literature on PsA clinical trial results for TNF antagonists studied in patients with similar clinical profiles at baseline (patient characteristics and response results are depicted in FIG. 8). Typical patients from all trials were analyzed, having the following characteristics: mean age=48 years, 66% with psoriasis, 60% male, 87 kg, 9 years duration of arthritis, 50% on concomitant MTX treatment. The odds ratios of treatment vs. placebo were used to control for a consistent placebo effect.

Health-related quality of life (HRQoL) is an appropriate measure of treatment benefits in PsA, given disease impact on this endpoint, the importance of this measure in therapeutic goals, and the representation of both psoriasis and joint components of disease by this endpoint. The SF-6D was used to measure determinants of HRQoL, and to estimate the effect of treatment with each TNF antagonist. Analysis showed that a 1.0 improvement in the HAQ corresponded to a 0.3 improvement in health utility, while a 1.0 improvement in PASI corresponded to a 0.1 improvement in health utility. These results indicate that change in PASI is mostly associated with the mental component of HRQoL.

To determine the cost of each TNF antagonist, weighted average costs (WACs) were used to represent the cost most closely related to the actual purchase price. Average wholesale prices (AWPs) were explored in a sensitivity analysis (Results are presented in Table 37). The drug costs for infliximab were more expensive than those associated with adalimumab or etanercept for the first six months of treatment, and were less expensive thereafter. When the cost of administration is included, however, infliximab was always the most expensive therapy.

TABLE 37
Costs of Medication (US Dollars)

| | | Unit | Cost- | Cost- |
|---|---|---|---|---|
| | Treatment Regimen | Cost | First 6 | Next 6 |
| Therapy | (Unit Size) | (WAC^{λ}) | months | months |
| Adalimumab | 40 mg; biweekly (40 mg) | 575 | 7477 | 7477 |
| Etanercept | 25 mg; twice weekly (25 mg) | 144 | 7477 | 7477 |
| Infliximab | 5 mg/kg; 0, 2, 6, then every 8 | 532 | 10640 | 6916 |
| | weeks (100 mg) | | | |

^{λ}Weighted Average Cost - more closely related to actual purchase price (updated July 2005).
*Assumes 4 vials used 5 times in the first 6 months, and 6.5 (52/8) per year thereafter.

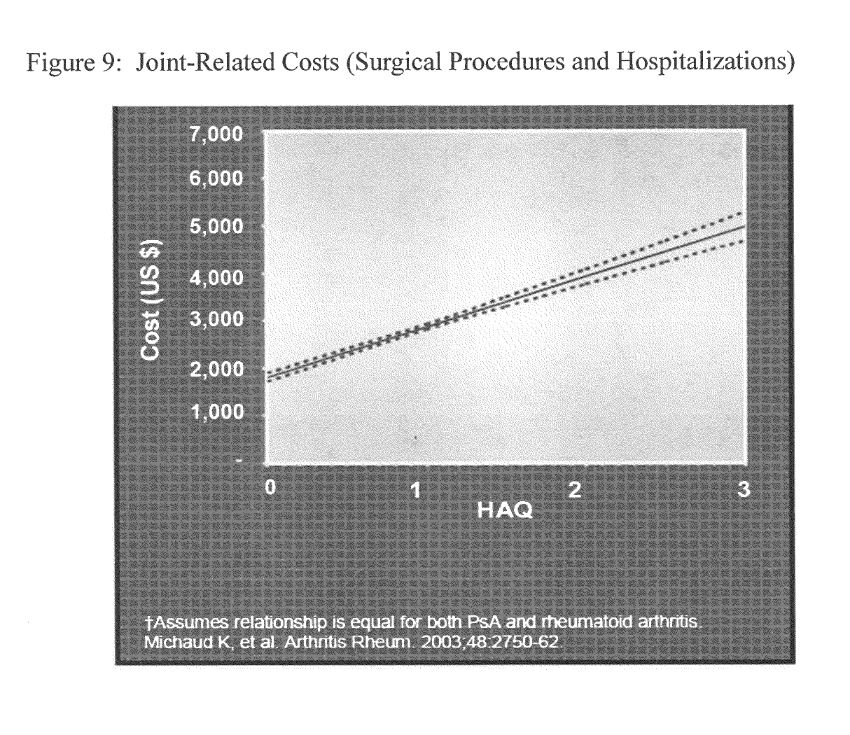

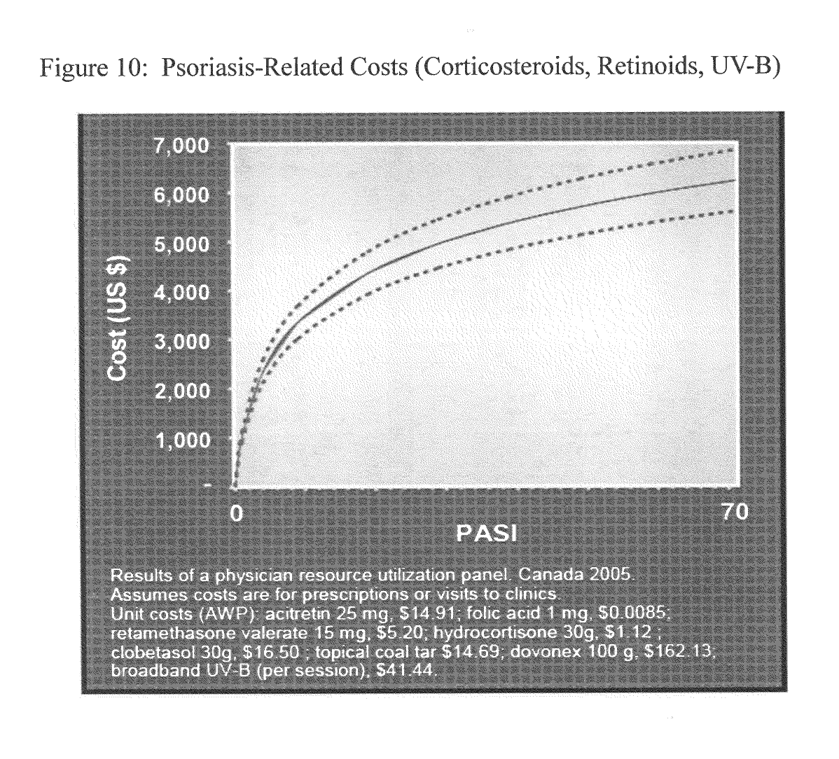

Other direct costs, including those associated with joint-related and psoriasis-related conditions, are important as well. Costs were estimated based on disability (HAQ) and psoriasis severity (PASI) for each treatment (FIGS. 9, 10).

Cost-consequence analysis was developed for a 5-year time horizon from a US managed care perspective. Duration of therapy was estimated using response decision rules and/or data from a 5,000 patient, long-term biologics registry (BIOBASDER) [http://biobadaser.ser.es/]. HRQoL and length of life were combined into quality-adjusted life-years (QALYs). Future costs and health benefits were discounted at 3% per year. The model simulated beyond the length of trials, using extrapolations from open-label data for patients remaining on treatment, and data from a long-term prospective cohort study conducted at the Psoriatic Arthritis Clinic, located in Toronto [http://www.uhnres.utoronto.ca/studies/cpsrd/].

### Results

The cost (US Dollars) and efficacy of each TNF antagonist were estimated for a patient population with the following baseline characteristics: 50% male, age=50 years, disease duration=10 years, HAQ=1.0, and PASI=8.0 in patients with psoriasis body surface area (BSA)>3% (all mean values expect % male). Mean baseline SF-6D was 0.66 (0.65-0.68). Relative to methotrexate, all three TNF antagonists demonstrated significant improvement in SF-6D, with adalimumab and infliximab yielding the greatest improvements in patients with both active components of disease. The cost of treatment for infliximab at 24 weeks was significantly higher than etanercept and adalimumab.

A management strategy scenario was presented where patients remain on treatment until they withdraw as a result of safety concerns or loss of efficacy. The estimated total costs and QALYs at 5 years are presented in Table 38.

TABLE 38
Estimated Total Costs and QALYs at 5 Years*

| | Total Cost | Total QALY |
|---|---|---|
| Etanercept | $80,981 | 2.34 |
| Adalimumab | 78,599 | 2.41 |
| Infliximab | 83,198 | 2.46 |
| DMARD | 38,136 | 1.97 |

*In patients with 60% active psoriasis at baseline

The additional impacts of adalimumab and infliximab on the psoriasis component of disease mean that these treatments are estimated to give more QALYs than etanercept. Both treatments are estimated to save costs through improvement in psoriasis (results not shown). Infliximab is the most expensive treatment, because of its high medication and administration costs.

Assuming that patients from different trials are comparable, indirect cost-effectiveness ratios can be estimated. Due to the uncertainty in many parameters, probabilistic sensitivity analyses were conducted to estimate the probability each TNF antagonist was the most cost-effective strategy (Results are presented in Table 39).

TABLE 39
Probability Each TNF Antagonist was the Most Cost-Effective*

| Intervention | Etanercept | Adalimumab | Infliximab |
|---|---|---|---|
| Baseline | 18% | 62% | 20% |
| Use AWP prices for | 24% | 48% | 28% |
| medication costs | | | |
| 25% fewer patients with | 40% | 34% | 26% |
| psoriasis at baseline than | | | |
| base case | | | |
| 100% patients with psoriasis | 0% | 90% | 10% |
| at baseline | | | |
| Assume only 3 vials of infliximab | 2% | 2% | 96% |
| (patient <60 kg^{†}) | | | |
| Assume 5 vials of infliximab | 20% | 80% | 0% |
| (patient >80 kg^{†}) | | | |
| No psoriasis-related costs | 44% | 50% | 6% |
| No disability-related costs | 20% | 60% | 20% |

*Assumes a cost-effectiveness ratio of $50,000 per QALY [Eichler HG, et al., Value Health. 2004; 7: 518-28].
^{†}Average weight of patients with PsA in etanercept and adlimumab clinical trials was 83 kg.

### Conclusions

With increasing health care costs, the focus of new research is often on how to allocate funds in the most efficient way, with benefits maximized for given budgets. With head-to-head studies of TNF antagonists unlikely, economic modeling is important to policy makers. Differential effects of treatment in psoriasis have important consequences for estimated costs and benefits (QALYs). The results presented herein demonstrate that TNF antagonists have different levels of effectiveness, as measured by the SF-6D health utility. Adalimumab appeared to provide superior efficacy compared with etanercept in patients with both skin and joint involvement, and comparable efficacy to infliximab at 63% of the cost. Adalimumab and infliximab appear to treat both psoriasis and joint disease most effectively. With available evidence, the probability adalimumab is the most cost-effective TNF antagonist for patients with PsA is relatively high. Clinicians and policy-makers should consider both the impact of costs and health utility consequences to guide their choice of TNF inhibitor for PsA treatment.

### Example 8

### Efficacy of Adalimumab in Psoriatic Arthritis as Measured by the Disease Activity Score 28 (DAS28)

Psoriatic arthritis (PsA) is an inflammatory arthropathy that can lead to progressive joint destruction in some patients and is associated with elevated tumor necrosis factor (TNF) concentrations in skin lesions and joints. Adalimumab is a fully human anti-TNF monoclonal antibody that is approved for treatment of PsA in the US and Europe, and for treatment of moderately to severely active rheumatoid arthritis (RA) in adults in the US, Europe and elsewhere. Study G has demonstrated adalimumab to be a safe and efficacious treatment for patients with psoriatic arthritis (PsA). The 28-joint Disease Activity Score (DAS28) is a continuous measure of arthritis activity that is validated and widely used in RA, but has received limited use in PsA. The objective of the study described herein was to determine the efficacy of adalimumab in PsA using the CRP-based DAS28 scale, the ACR component scores, and other measures. This analysis evaluates DAS28 responses in Study G in conjunction with components of the ACR core criteria.

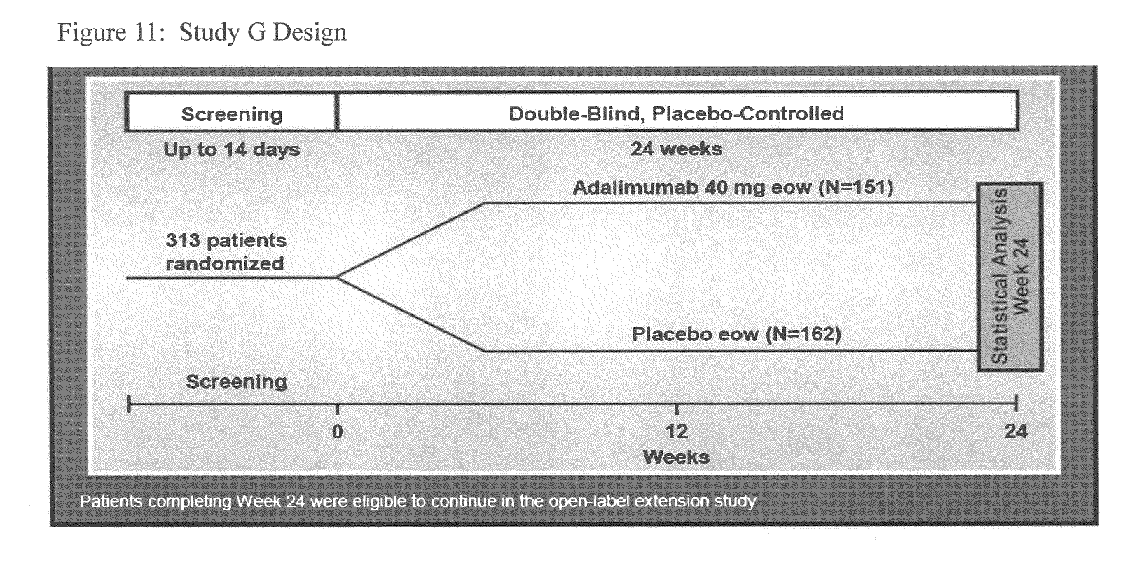

Study G is a Phase III, randomized, parallel, placebo-controlled, double-blind trial conducted in the US, UK, Canada, France, Germany, Belgium, Italy, and Austria. The Study G study design is outlined in FIG. 11. Patients were randomized 1-to-1 to receive, subcutaneously administered, placebo or adalimumab 40 mg every other week (eow). Randomization was centrally stratified by methotrexate (MTX) use (yes/no) and extent of psoriasis (<3% or ≧3% body surface area [BSA]) at baseline. Inclusion criteria were the following (selected): ≧3 swollen and ≧3 tender joints, inadequate response to NSAID therapy, history of psoriasis, and age≧18 years. Exclusion criteria included (selected): prior anti-TNF therapy, prior Alefacept (within 12 weeks), prior other biologics (within 6 weeks), prior DMARDs except MTX (within 4 weeks), prior systemic therapies for psoriasis (within 4 weeks), and prior phototherapy and topicals (within 2 weeks). Enrollment screening included chest x-ray, electrocardiogram, PPD skin test, and routine laboratory tests.

Patients were allowed to receive rescue therapy with steroids or DMARDs following the Week 12 evaluation if they failed to have a 20% decrease from baseline in both the swollen and tender joint counts for 2 consecutive visits. Study visits were at Weeks 2, 4, and then every 4 weeks until Week 24. Efficacy measures included: ACR response criteria (co-primary endpoint: ACR20 response at Week 12); radiographic (mean change in modified Total Sharp Score at Week 24, Mease et al, Arthritis Rheum. 2005; 52:3279-3289); modified Psoriatic Arthritis Response Criteria (PsARC) response rates; Health Assessment Questionnaire Disability Index (HAQ); Psoriasis Area and Severity Index (PASI) in patients with psoriasis affecting ≧3% body surface area (BSA) at study entry; and Physician's Global Assessment (PGA) of psoriasis. ACR components were evaluated from each visit with CRP-based DAS28 being calculated post-hoc. Statistical data analyses were performed on the intent-to-treat population. ACR and PASI scores were analyzed by non-responder imputation. All other measures were analyzed by last observation carried forward (LOCF).

### Results

313 patients (151 adalimumab, 162 placebo) enrolled in Study G, and 289 (92%) completed the 24-week study. According to the standard in the art (see Mease et al. (2005) Annals of the Rheumatic Diseases 64:ii49-ii54), DAS28 scores were determined according to the following formula:

[0.56×√TJC28+0.28×√SJC28+0.36×In(CRP+1)+0.014×GH+0.96].

A DAS28 of ≦3.2 is considered low/mild, >3.2 to ≦5.1 is considered moderate, and >5.1 is considered severe disease activity. Clinical remission is considered a DAS28 score of less than 2.6. Baseline demographics (shown in Table 40) and mean DAS28 scores were comparable between the placebo and adalimumab treatment groups, as well as with moderate to severe PsA, with 40% of adalimumab patients and 41% of placebo patients meeting the RA definition for severe disease (DAS28>5.1). Approximately half of all patients received concomitant MTX.

TABLE 40
Baseline Demographics and Disease Characteristics

| | Placebo | Adalimumab |
|---|---|---|
| | eow | 40 mg eow |
| Characteristic* | N = 162 | N = 151 |
| Age (years) | 49.2 ± 11.1 | 48.6 ± 12.5 |
| % Male | 54.9 | 56.3 |
| % Caucasian | 93.8 | 97.4 |
| Body Weight (kg) | 85.5 ± 16.5 | 86.0 ± 20.6 |
| Rheumatoid Factor negative (%) | 90.1 | 89.4 |
| Duration of Psoriatic Arthritis (years) | 9.2 ± 8.7 | 9.8 ± 8.3 |
| Duration of Psoriasis (years) | 17.1 ± 12.6 | 17.2 ± 12.0 |
| No. of previous DMARDs | 1.5 ± 1.2 | 1.5 ± 1.2 |
| DAS28 | 4.9 ± 1.1 | 4.8 ± 1.1 |

*Mean values ± SD, except percentages

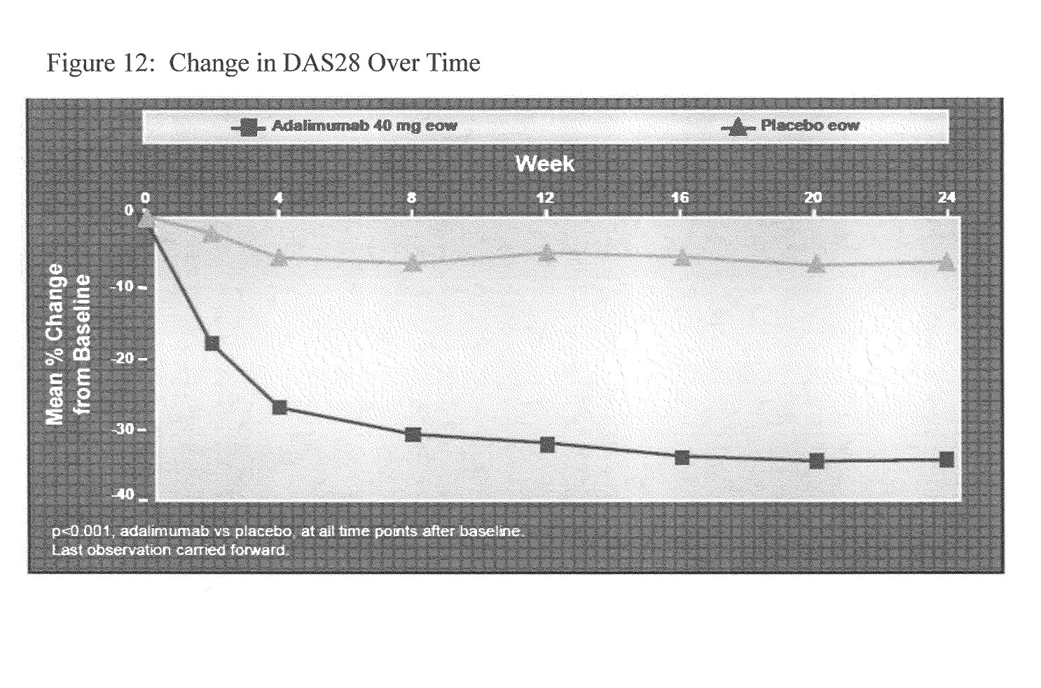

Mean percentage improvement from baseline in DAS28 scores was significantly greater in patients treated with adalimumab versus placebo from Week 2-24, as shown in FIG. 12. Treatment with adalimumab led to a marked increase in the number of patients with mild disease activity and a 66% decrease in the number with severe disease activity, as shown in Table 41.

TABLE 41
Percentage of Patients with Mild or Severe Arthritis
Disease Activity at Baseline and Week 24

| | % of Patients | | | |
|---|---|---|---|---|
| | Mild | | Severe | |
| | DAS28 ≦3.2 | | DAS28 >5.1 | |
| | Baseline | Week 24 | Baseline | Week 24 |
| Placebo (N = 158) | 4 | 17 | 41 | 34 |
| Adalimumab (N = 148) | 4 | 57* | 40 | 14* |

*p < 0.001, adalimumab vs. placebo at Week 24.
Last observation carried forward.

A DAS28 score<2.6 (clinical remission) was achieved by a significantly greater percentage of patients treated with adalimumab versus placebo, as shown in Table 42.

TABLE 42
DAS28 <2.6 at Weeks 12 and 24

% of Patients

| | Week 12 | Week 24 |
|---|---|---|
| Placebo (N = 158) | 4 | 9 |
| Adalimumab (N = 148) | 37* | 41* |

*p < 0.001, adalimumab vs. placebo.
Last observation carried forward.

At Weeks 12 and 24, ACR 20/50/70 response rates were significantly higher with adalimumab than placebo, as shown in Table 43. The ACR score component measures were comparable between the adalimumab and placebo groups at baseline.

TABLE 43
ACR Responses at Weeks 12 and 24

| % of Patients | | |
|---|---|---|
| ACR20 | ACR50 | ACR70 |

Week 12

| | | | |
|---|---|---|---|
| Placebo (N = 162) | 14 | 4 | 1 |
| Adalimumab (N = 151) | 58* | 36* | 20* |

Week 24

| | | | |
|---|---|---|---|
| Placebo (N = 162) | 15 | 6 | 1 |
| Adalimumab (N = 151) | 57* | 39* | 23* |

*p < 0.001, adalimumab vs. placebo.
Non-responder imputation.

At Week 24, all component scores had improved significantly in the adalimumab group, as shown in Table 44.

TABLE 44
Mean Change in Efficacy Parameters at Week 24

| | | | Adalimumab | |
|---|---|---|---|---|
| | Placebo eow | | 40 mg eow | |
| | (N = 162) | | (N = 151) | |
| | | Mean | | Mean |
| | Baseline | Change | Baseline | Change |
| DAS28 | 4.9 | −0.3 | 4.8 | −1.7 |
| HAQ (0-3) | 1.0 | −0.1 | 1.0 | −0.4 |
| CRP (mg/dL) | 1.4 | 0.0 | 1.4 | −0.9 |
| TJC78 | 25.8 | −2.9 | 23.9 | −11.3 |
| SJC76 | 14.3 | −2.4 | 14.3 | −6.1 |
| Patients Assessment of Pain | 48.8 | 0.6 | 51.1 | −24.0 |
| (VAS mm) | | | | |
| Patients Global Assessment | 48.1 | 0.6 | 47.1 | −21.1 |
| (VAS mm) | | | | |
| Physician's Global | 53.5 | −6.4 | 53.8 | −31.3 |
| Assessment (VAS mm) | | | | |

*p ≦ 0.001, adalimumab vs. placebo for all variables.
Last observation carried forward.

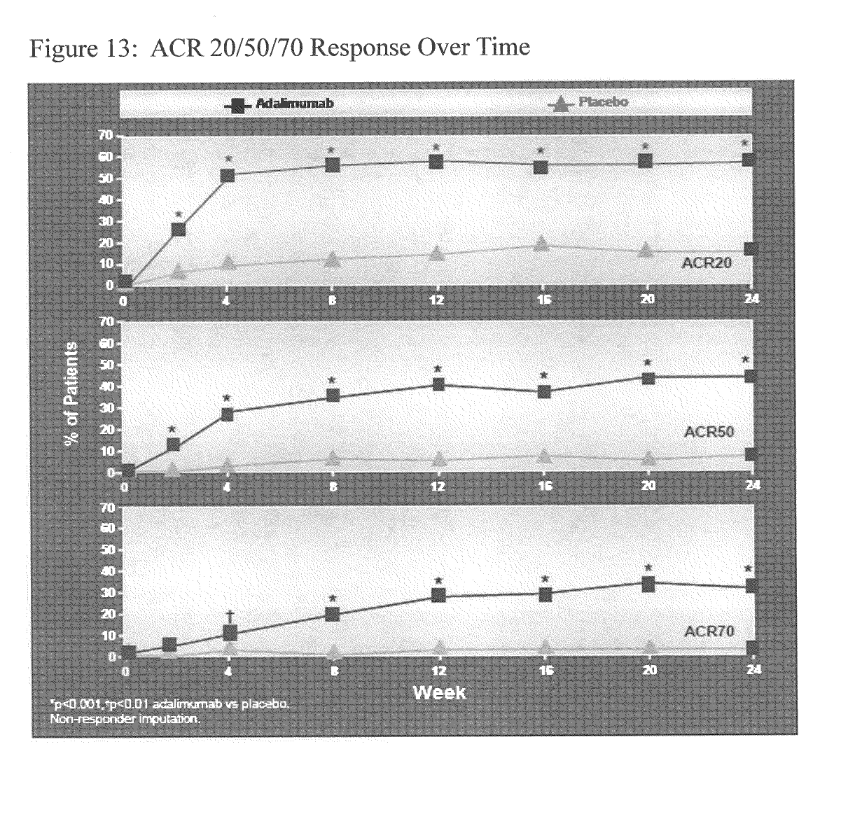

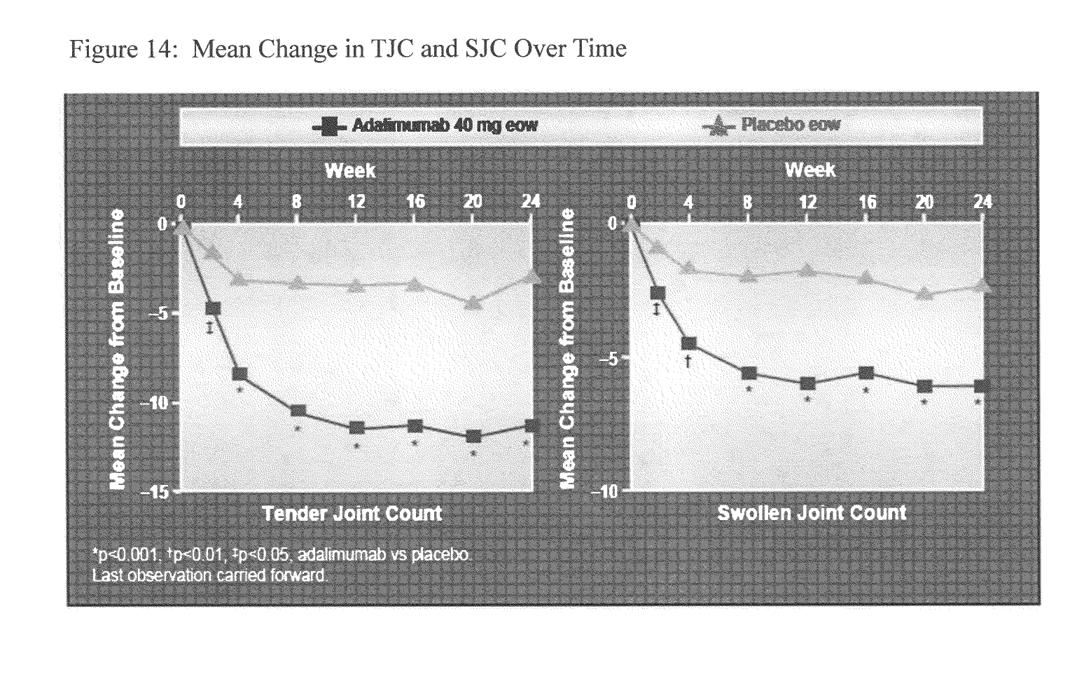

ACR responses were significantly better with adalimumab than placebo as early as Week 2 and out to Week 24. The percentages of patients achieving ACR20/50/70 were 57/39/23 for adalimumab and 15/06/01 for placebo, as shown in FIG. 13. Disability, as measured by HAQ, improved significantly in patients treated with adalimumab compared to placebo at Weeks 12 and 24, as shown in Table 45. Adalimumab treated patients achieved rapid and sustained improvements in Tender and Swollen Joint Counts, as shown in FIG. 14.

Adalimumab was generally well-tolerated, as previously reported in Study G. No significant changes in the safety parameters were observed over 24 weeks of adalimumab treatment. Common adverse events ≧5% at Week 24 are shown in Table 46. Elevation of ALT (≧3×ULN) was more common in adalimumab treated patients. The majority of patients were on concomitant hepatotoxins (mainly MTX) and had elevations that were transient, returning to normal while on the study drug. No cases of malignancy (including lymphoma), tuberculosis/granulomatous events, demyelination, or drug-induced lupus were observed in either treatment group.

TABLE 45
Mean Change in HAQ at Weeks 12 and 24

Mean Change from
Baseline

| | Week 12 | Week 24 |
|---|---|---|
| Placebo (N = 162) | −0.1 | −0.1 |
| Adalimumab (N = 151) | −0.4* | −0.4* |

*p < 0.001, adalimumab vs. placebo.
Last observation carried forward.
Minimum Clinically Important difference = −0.3 (Mease PJ, et al. Ann. Rheum. Dis. 2004; 63(Suppl 1): 391-392.

TABLE 46
Common Adverse Events ≧5% at Week 24

| | Placebo | Adalimumab |
|---|---|---|
| | eow | 40 mg eow |
| | N = 162 | N = 151 |
| | n (%) | n (%) |
| Any AE | 130 (80.2) | 122 (80.8) |
| Any SAE | 7 (4.3) | 5 (3.3) |
| Upper Respiratory Tract Infection NOS | 24 (14.8) | 19 (12.6) |
| Nasopharyngitis | 15 (9.3) | 15 (9.9) |
| Injection site reaction NOS | 5 (3.1) | 10 (6.6) |
| Headache NOS | 14 (8.6) | 9 (6.0) |
| Hypertension NOS | 5 (3.1) | 8 (5.3) |
| PsA aggravated | 11 (6.8) | 5 (3.3) |
| Ps aggravated | 10 (6.2) | 3 (2.0) |
| Diarrhea NOS | 9 (5.6) | 3 (2.0) |
| Arthralgia | 9 (5.6) | 3 (2.0) |

SAE = Serious adverse events.
NOS = Not otherwise specified.

### Conclusions

For patients with moderate to severe PsA, adalimumab was efficacious in improving several clinical parameters of disease activity, including ACR components and overall ACR response. DAS28 scores and DAS28 remission rates showed significant improvements in PsA patients treated with adalimumab. The changes observed in the DAS28 with adalimumab therapy suggest that this clinical measure may be applicable in PsA. Adalimumab was safe and well-tolerated during 24 weeks of PsA treatment.

### Example 9

### Clinical Efficacy and Safety of Adalimumab for Psoriatic Arthritis: 48-Week Results of Study G

Tumor necrosis factor (TNF) concentrations are elevated in skin lesions and joints in patients with psoriatic arthritis (PsA). Adalimumab is a fully human monoclonal antibody that targets TNF and inhibits the inflammatory process in PsA. Adalimumab has been shown to be efficacious in rheumatoid arthritis (RA) when used in combination with methotrexate (MTX), or as monotherapy. Study G demonstrated that adalimumab (ADA) is an effective treatment for the joint and skin disease of psoriatic arthritis for up to 24 weeks. The report presented herein describes the long-term effect of ADA on arthritis and psoriasis in PsA patients following an additional 24 weeks of therapy. The objective of the study described herein was to evaluate the 48-week efficacy and safety of adalimumab in patients with moderately to severely active PsA.

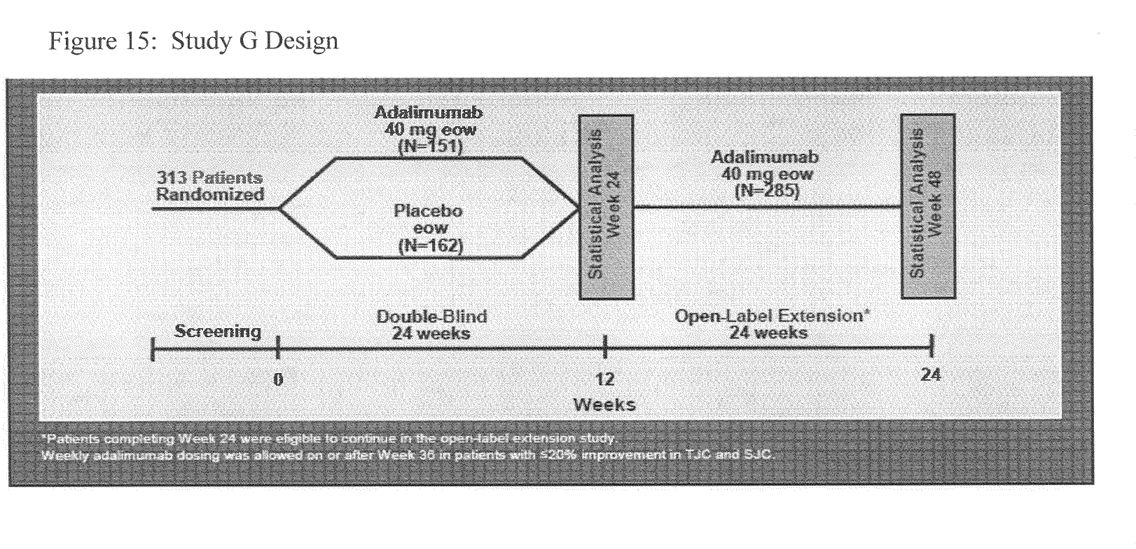

Study G is a Phase III, double-blind, randomized, placebo (PBO)-controlled study of patients with moderate to severe PsA (≧3 swollen and ≧3 tender joints) who have failed NSAID therapy. Additional inclusion criteria included a history of psoriasis and ≧18 years of age. Exclusion criteria included prior anti-TNF therapy. Patients were stratified according to methotrexate (MTX) use (yes/no) and extent of psoriasis (<3% and ≧3% BSA), and randomized to receive ADA 40 mg or PBO every other week (eow) for 24 weeks (the Study G study design, including the open-label extension, is outlined in FIG. 15). Patients completing the 24-week trial were eligible to enroll in an open-label extension (OLE) study, during which all patients received ADA 40 mg eow. Following 12 weeks of open-label therapy, patients with an inadequate response were eligible to increase ADA to 40 mg every week. Primary measures were signs/symptoms (ACR 20 response at 12 weeks), and the mean change in modified Total Sharp Score at Week 24 (Mease et al, Arthritis Rheum. 2005; 52:3279-3289). Selected secondary measures included signs/symptoms (ACR 20/50/70), psoriasis (in patients with ≧3% BSA; PSAI and PGA), and quality of life, function, and fatigue (SF-36, HAQ-DI, and FACIT).

Data analyses were performed on the intent-to-treat population. ACR and PASI scores were analyzed by non-responder imputation. All other measures were analyzed by last observation carried forward. Adalimumab patients were analyzed as one cohort across 48 weeks. Placebo patients were analyzed as separate cohorts in Weeks 1-24 and Weeks 24-48.

### Results

313 patients were randomized to Study G and 285 continued into OLE. Baseline data were consistent with moderate to severe PsA and patients were well-matched between treatments. Baseline demographics and disease characteristics among the two treatment groups are shown in Table 47. Baseline disease characteristics are shown in Table 48. Withdrawals occurred at low rates and for similar reasons in both portions of the study. The disposition of patients is shown in Table 49.

TABLE 47
Baseline Demographics and Disease Characteristics

| | Placebo | Adalimumab |
|---|---|---|
| | eow | 40 mg eow |
| Characteristic* | N = 162 | N = 151 |
| Age (years) | 49.2 ± 11.1 | 48.6 ± 12.5 |
| % Male | 54.9 | 56.3 |
| % Caucasian | 93.8 | 97.4 |
| Body Weight (kg) | 85.5 ± 16.5 | 86.0 ± 20.6 |
| Rheumatoid Factor Negative (%) | 90.1 | 89.4 |
| Duration of Psoriatic Arthritis (years) | 9.2 ± 8.7 | 9.8 ± 8.3 |
| Duration of Psoriasis (years) | 17.1 ± 12.6 | 17.2 ± 12.0 |
| No. of previous DMARDs | 1.5 ± 1.2 | 1.5 ± 1.2 |
| % MTX use | 50.0 | 51.0 |

*mean values ± SD, except percentages

TABLE 48
Baseline Disease Characteristics

| | Placebo | Adalimumab |
|---|---|---|
| Characteristic* | eow | 40 mg eow |
| | N = 162 | N = 151 |
| Swollen Joint Count (0-76) | 14.3 ± 11.1 | 14.3 ± 12.2 |
| Tender Joint Count (0-78) | 25.8 ± 18.0 | 23.9 ± 17.3 |
| C-Reactive Protein (mg/dL) | 1.4 ± 1.7 | 1.4 ± 2.1 |
| HAQ (0-3) | 1.0 ± 0.7 | 1.0 ± 0.6 |
| | N = 69† | N = 70† |
| PASI (0-72) | 8.3 ± 7.3 | 7.4 ± 6.1 |
| (Range) | (0.4-40.9) | (0.2-38.0) |
| PGA (“Clear” or “Almost Clear” | 1 (1.4%) | 1 (1.4%) |

*Mean values ± SD, except percentages
†Patients with BSA ≧3%; N = 69 for PASI scores of adalimumab-treated patients

TABLE 49
Disposition of Patients

| | Double-blind | | Open-Label |
|---|---|---|---|
| | Weeks 0-24 | | Weeks 24-48 |
| | Placebo | Adalimumab | Adalimumab |
| | eow | 40 mg eow | 40 mg eow |
| | n (%) | n (%) | n (%) |
| Patients entering study | 162 | 151 | 285 |
| Patients completing study | 149 (92.0) | 140 (92.7) | 272 (95.4) |
| Patients prematurely | 13 (8.0) | 11 (7.3) | 13 (4.6) |
| terminated | | | |
| Primary reason for | | | |
| termination: | | | |
| Adverse event | 1 (0.6) | 3 (2.0) | 2 (0.7) |
| Unsatisfactory therapeutic | 4 (2.5) | 1 (0.7) | 3 (1.1) |
| effect | | | |
| Other | 1 (0.6) | 1 (0.7) | 4 (1.4) |

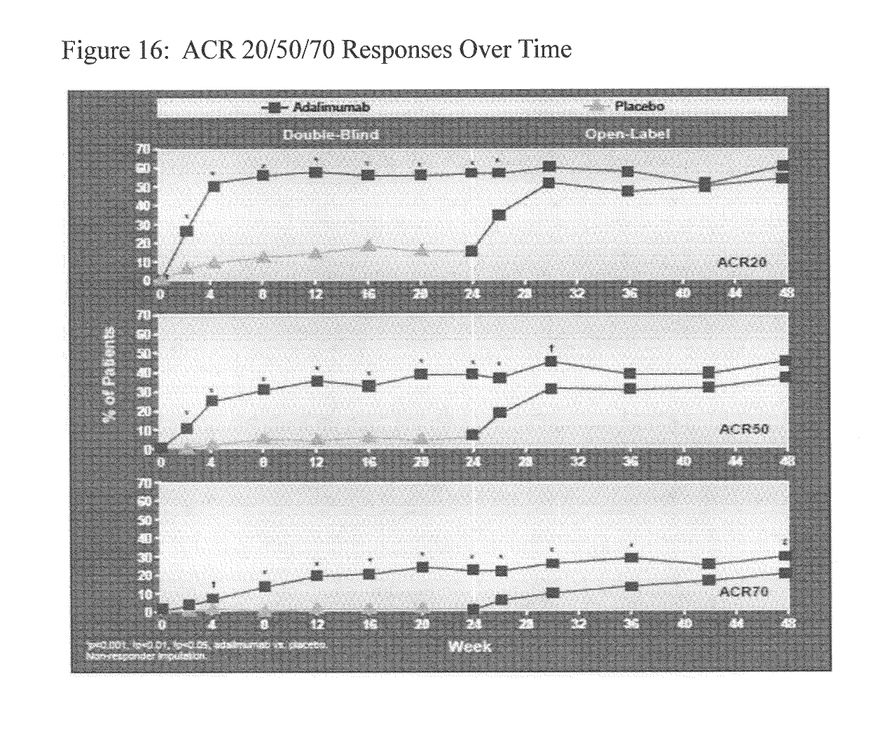

Among patients initially randomized to ADA, the 24-week improvements in ACR, PASI and HAQ scores were maintained to week 48. Similar responses were achieved by placebo patients during open-label treatment with adalimumab. ACR responses at weeks 24 and 48 are shown in Table 50. ACR 20/50/70 responses over time are shown in FIG. 16.

TABLE 50
ACR Responses at Weeks 24 and 48

| | % of Patients | | |
|---|---|---|---|
| | ACR20 | ACR50 | ACR70 |
| Week 24 (Double-Blind) | | | |
| Placebo (N = 162) | 15 | 6 | 1 |
| Adalimumab (N = 151) | 57* | 39* | 23* |
| Week 48 (Open-Label) | | | |
| Placebo/Adalimumab (N = 147) | 54 | 37 | 21 |
| Adalimumab (N = 151) | 61 | 46 | 31 |

*p < 0.001, adalimumab vs. placebo.
Non-responder imputation.
Twelve patients escalated to weekly adalimumab at Week 38, of whom 3 (25%) achieved an ACR20 response at Week 48.

The improvement in HAQ score achieved during the first 24 weeks of adalimumab treatment was maintained out to 48 weeks. A similar response was seen in placebo patients when treated with adalimumab from Weeks 24-48 (data is shown in Table 51). The mean change in HAQ at Weeks 24 and 48 is shown in Table 52.

TABLE 51
ACR, PASI, and HAQ Scores in ADA and PBO/ADA Treatment
Groups at Weeks 24 and 48

| | ADA | | PBO/ADA | |
|---|---|---|---|---|
| | Week 24 | Week 48 | Week 24 | Week 48* |
| | N = 151 | N = 151 | N = 162 | N = 147 |
| ACR20/50/70 | 57/39/23*** | 61/46/31 | 15/6/1 | 54/37/21 |
| HAQ mean Δ | −0.4*** | −0.4 | −0.1 | − |
| | N = 69 | N = 69 | N = 69 | N = 59 |
| PASI 50/70/90 | 75/59/42*** | 70/58/46 | 12/1/0 | 76/63/47 |
| PASI mean % Δ | −66*** | −67 | 24 | −72 |

*Received ADA after week 24. Patients who prematurely discontinued prior to receiving ADA are not included in this analysis.
***p < 0.001 vs. PBO at week 24

TABLE 52
Mean Change in HAQ at Weeks 24 and 48

| | Mean Change From Baseline |
|---|---|
| Week 24 (Double-Blind) | |
| Placebo | −0.1 |
| Adalimumab | −0.4* |
| Week 48 (Open-Label) | |
| Placebo/Adalimumab | −0.4 |
| Adalimumab | −0.4 |

*p < 0.001, adalimumab vs. placebo.
Last observation carried forward.
Minimum Clinically Important difference = −0.3 (Mease PJ, et al. Ann. Rheum. Dis. 2004; 63(Suppl 1): 391-392.

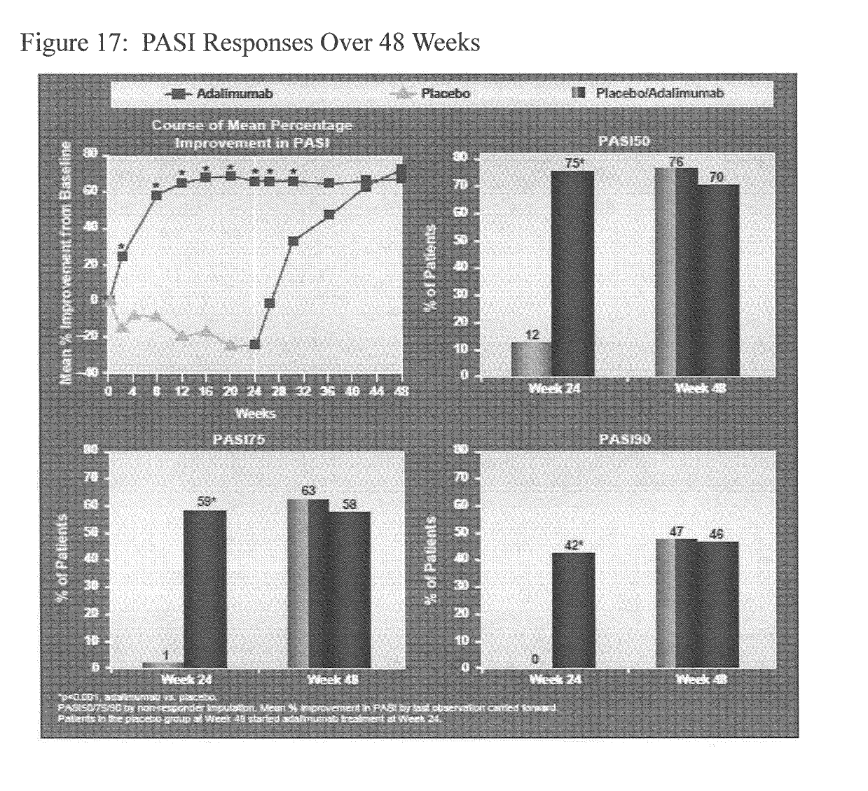

PASI responses had rapid onset and were maintained out to Week 48, when approximately half of adalimumab patients had achieved a PASI90 response. PASI responses over 48 Weeks are shown in FIG. 17.

A PGA of Clear or Almost Clear was achieved by about two-thirds of patients after 24 weeks of adalimumab, and this response was maintained out to Week 48 (data is shown in Table 53).

TABLE 53
Physician Global Assessment: Clear or Almost Clear at Weeks 24 and 48

| | % of Patients^{†} |
|---|---|
| Week 24 (Double-Blind) | |
| Placebo (N = 69) | 10 |
| Adalimumab (N = 70) | 67* |
| Week 48 (Open-Label) | |
| Placebo/Adalimumab (N = 69) | 57 |
| Adalimumab (N = 70) | 63 |

*p < 0.001, adalimumab vs. placebo.
Last observation carried forward.
^{†}Percentage of patients who at Week 24 or 46 had a PGA assessment of Clear or Almost Clear.

Baseline use of MTX did not significantly affect ACR or PASI response rates following 48-week treatment with adalimumab (PASI responses were slightly higher in patients taking concomitant MTX, but this difference was not statistically significant; data is shown in Table 54).

TABLE 54
ACR and PASI Responses by MTX Use

| | % of Patients | | |
|---|---|---|---|
| | ACR20 | ACR50 | ACR70 |
| Adalimumab without MTX | 58 | 43 | 31 |
| (N = 74) | | | |
| Adalimumab with MTX (N = 77) | 62 | 48 | 31 |
| | PASI50 | PASI70 | PASI90 |
| Adalimumab without MTX | 63 | 50 | 40 |
| (N = 40) | | | |
| Adalimumab with MTX (N = 29) | 70 | 69 | 55 |

p > 0.05 for all comparisons between adalimumab with MTX vs. without MTX.
Non-responder imputation.

Thirty patients increased ADA at week 36, to 40 mg weekly. ADA was generally safe and well-tolerated through Week 48. The safety profile during the open-label therapy was consistent with that reported for the initial 24 weeks, and that described for ADA in RA studies. The common adverse events that occurred in ≧5% of patients in the double-blind trial are shown in Table 55.

TABLE 55
Common Adverse Events That Occurred in ≧5% of Patients in the
Double-Blind Trial

| | Double-Blind | | Open-Label |
|---|---|---|---|
| | Week 0-24 | | Week 24-48 |
| | Placebo | Adalimumab | Adalimumab |
| | eow | 40 mg eow | 40 mg eow |
| | N = 162 | n = 151 | n = 285 |
| | n (%) | n (%) | n (%) |
| Any AE | 130 (80.2) | 122 (80.8) | 226 (79.3) |
| Any SAE | 7 (4.3) | 5 (3.3) | 11 (3.9) |
| Upper respiratory tract | 24 (14.8) | 19 (12.6) | 39 (13.7) |
| infection NOS | | | |
| Nasopharyngitis | 15 (9.3) | 15 (9.9) | 31 (10.9) |
| Injection site reaction NOS | 5 (3.1) | 10 (6.6) | 24 (8.4) |
| Headache NOS | 14 (8.6) | 9 (6.0) | 18 (6.3) |
| Hypertension NOS | 5 (3.1) | 8 (5.3) | 12 (4.2) |
| PsA aggravated | 11 (6.8) | 5 (3.3) | 10 (3.5) |
| Ps aggravated | 10 (6.2) | 3 (2.0) | 3 (1.1) |
| Diarrhea NOS | 9 (5.6) | 3 (2.0) | 6 (2.1) |
| Arthralgia | 9 (5.6) | 3 (2.0) | 10 (3.5) |

SAE = Serious adverse events
NOS = Not otherwise specified

### Conclusions

Forty-eight weeks of treatment with adalimumab was efficacious against arthritis and skin disease of PsA patients in Study G. ADA therapy was effective in treating the signs and symptoms of PsA with significant reductions in the burden of joint disease, skin disease, and disability for a one-year period. Rates of individual adverse events and serious adverse events were comparable between adalimumab and placebo. Adalimumab demonstrated a good safety profile and was well-tolerated by PsA patients.

### Example 10

### Inhibition of Joint Destruction in PsA with Adalimumab: 48-Week Results of Study G

Erosive polyarthritis occurs in the joints of a large proportion of patients with psoriatic arthritis (PsA). Traditional non-biologic DMARDs have not been shown to be effective in inhibiting radiographic progression of joint damage in PsA. Adalimumab is a fully human monoclonal anti-TNF antibody that has been shown to inhibit radiographic progression of joint damage in rheumatoid arthritis (RA).

Study G was a Phase III, randomized, parallel, placebo-controlled, double-blind trial conducted in a number of countries. Study G was a 24-week blinded trial that has demonstrated the efficacy of adalimumab against signs, symptoms and radiographic progression of arthritis in patients with moderately to severely active PsA (Mease et al, Arthritis Rheum. 2005; 52:3279-3289). Upon study completion, patients had the option of progressing into an open-label extension trial (study design is outlined in FIG. 15). The objective of the report described herein was to evaluate the 48-Week efficacy of adalimumab in inhibiting radiographic progression of psoriatic joint disease in patients enrolled in the open-label extension of Study G.

Patients were randomized 1-to-1 to receive subcutaneously administered placebo or adalimumab 40 mg every other week (eow). Randomization was centrally stratified by methotrexate (MTX) use (Yes/No) and extent of psoriasis (<3% or ≧3% body surface area [BSA]) at baseline. Selected inclusion criteria were the following: ≧3 swollen and ≧3 tender joints, inadequate response to NSAID therapy, a history of psoriasis, and age≧18 years. Selected exclusion criteria were: prior anti-TNF therapy, prior Alefacept (within 12 weeks), prior other biologics (within 6 weeks), prior DMARDs except MTX (within 4 weeks), prior systemic therapies for psoriasis (within 4 weeks), and prior phototherapy and topicals (within 2 weeks). Enrollment screening included chest x-ray, electrocardiogram, PPD skin test, and routine laboratory tests. Patients were allowed to receive rescue therapy with steroids or DMARDs following the Week 12 evaluation if they failed to have a 20% decrease from baseline in both the swollen and tender joint counts for 2 consecutive visits.

Study visits were conducted at Weeks 2, 4, and then every 4 weeks until Week 24. Study G primary measures were: ACR20 response at 12 weeks (signs/symptoms), and mean change in modified Total Sharp Score (mTSS) at Week 24 (radiographic; see Mease et al, Arthritis Rheum. 2005; 52:3279-3289). Study G secondary measures were: ACR 20/50/70 (signs/symptoms); PASI, DLQI, and PGA (psoriasis, in patients with >3% BSA); SF-36, HAQ-DI, and FACIT (quality of life, function, and fatigue).

Radiographic assessments were performed during the blinded (Weeks 0 and 24) and open-label (Week 48) portions of the study. Radiographs of hands & feet were assessed by an mTSS that included additional joints typically involved in PsA and used expanded numerical scales to better quantify osteolysis. Two readers experienced with radiography in PsA who were blinded to treatment and film order evaluated radiographs.

As a radiographic scoring method, the Modified Total Sharp Score (mTSS) was determined according to the following criteria: joint space narrowing was assessed at 48 sites, each site receiving a score between 0-4, and erosion was assessed at 54 sites, each site receiving a score between 0-7. The range of possible scores for joint space narrowing was consequently 0-192, and the range of possible scores for erosion was 0-378. The sum of these values determined the mTSS, which could range from 0-570. Other radiographic findings associated with PsA include phalangeal tuft resorption (measurable at 12 sites), subluxation (26 sites), pencil-in-cup (18 sites), periostitis (38 sites), and juxta-articular periostitis (52 sites). PsA-associated findings were also assessed.

Inclusion in the Week 48 radiographic analysis required both baseline and Week 24 films. If a Week 48 film was not available, a Week 48 score was obtained by linear imputation from baseline and Week 24 for patients randomized to adalimumab, and by last observation carried forward from Week 24 for patients randomized to placebo.

Statistical data analyses were performed on the intent-to-treat population. ACR scores were analyzed by non-responder imputation. All other measures were analyzed by last observation carried forward. Adalimumab patients were analyzed as one cohort across 48 weeks. Placebo patients were analyzed as separate cohorts in Weeks 1-24 and Weeks 24-48. Radiographic outcomes were assessed by comparing the change in mTSS in adalimumab patients at Week 48 with placebo patients at Week 24.

### Results

Baseline data were consistent with moderate to severe PsA and were well-matched between arms (baseline demographics and disease characteristics are shown in Table 56). BL values for mTSS, ERO, and JSN in ADA vs. PBO patients were 22.7 vs. 19.1, 11.4 vs. 10.0, and 11.2 vs. 9.2, respectively.

TABLE 56
Baseline Demographics and Disease Characteristics

| | | Adalimumab 40 mg |
|---|---|---|
| | Placebo eow | eow |
| | N = 162 | N = 151 |
| Age (years) | 49.2 ± 11.1 | 48.6 ± 12.5 |
| Sex (% Male) | 54.9 | 56.3 |
| Race (% Caucasian) | 93.8 | 97.4 |
| Duration of psoriatic arthritis | 9.2 ± 8.7 | 9.8 ± 8.3 |
| (years) | | |
| Duration of psoriasis (years) | 17.1 ± 12.6 | 17.2 ± 12.0 |
| Swollen Joint Count (0-76) | 14.3 ± 11.1 | 14.3 ± 12.2 |
| Tender Joint Count (0-78) | 25.8 ± 18.0 | 23.9 ± 17.3 |
| HAQ (0-3) | 1.0 ± 0.7 | 1.0 ± 0.6 |
| | N = 141 | N = 133 |
| mTSS | 22.1 ± 39.2 | 23.4 ± 44.8 |
| Joint space narrowing | 10.4 ± 18.3 | 11.0 ± 20.9 |
| Erosions | 11.8 ± 22.0 | 12.4 ± 25.0 |

Mean values ± SD except percentages

Withdrawal rates were low in the blinded and open-label portions of the study. The disposition of patients is shown above in Table 49.

ACR response rates in adalimumab-treated patients were maintained and slightly improved out to 48 weeks. Similar ACR response rates were achieved by placebo patients following 24 weeks of open-label treatment with adalimumab (data is shown in Table 57).

TABLE 57
ACR Responses at Weeks 24 and 48

| | % of Patients | | |
|---|---|---|---|
| | ACR20 | ACR50 | ACR70 |
| Week 24 (Double-Blind) | | | |
| Placebo (N = 162) | 15 | 6 | 1 |
| Adalimumab (N = 151) | 57* | 39* | 23* |
| Week 48 (Open-Label) | | | |
| Placebo/Adalimumab (N = 147) | 54 | 37 | 21 |
| Adalimumab (N = 151) | 61 | 46 | 31 |

*p < 0.001, adalimumab vs. placebo.
Non-responder imputation.

For patients randomized to ADA, 144 had BL and Week 24 films, and 128 had Week 48 films. For patients randomized to PBO, 152 had BL and Week 24 films, and 134 had Week 48 films.

Patients treated with adalimumab for 48 weeks (Study G+24 weeks open-label trial) demonstrated less radiographic progression than patients who received 24 weeks of placebo. Cumulative function plots revealed that 15% of patients had progression on adalimumab, while 24% had progression on placebo (progression was considered a change in mTSS>0.5). A statistically significant difference in the mean change in mTSS through Week 48 was observed between the treatment groups, as is shown in Table 58.

TABLE 58
Mean Change in mTSS Through Week 48

| | | | Week 24 | Week 48 |
|---|---|---|---|---|
| | N | Baseline | Mean Change | Mean Change |
| Placebo | 141 | 22.1 | 0.9 | 1.0 |
| Adalimumab | 133 | 23.4 | −0.1 | 0.1* |

*p < 0.001, adalimumab (Week 48) vs. placebo (Week 24).

The change in mTSS by category of treatment is shown in Table 59. No significant changes from baseline were found in phalangeal tuft resorption, periostitis, or other PsA-associated findings in patients treated with adalimumab over 48 weeks. Less radiographic progression was observed following 48 weeks of treatment with adalimumab compared with 24 weeks of placebo, both in patients who were receiving concomitant MTX at baseline and in those who were not (data is shown in Table 60).

TABLE 59
Change in mTSS by Category

| | Placebo (Week 24) | Adalimumab (Week 48) |
|---|---|---|
| | N = 141 | N = 133 |
| | N (%) | N (%) |
| Decrease in mTSS | 8 (5.7) | 25 (18.8) |
| (Δ < 0.5) | | |
| No change in mTSS | 99 (70.2) | 88 (66.2) |
| Increase in mTSS | 34 (24.1) | 20 (15.0) |
| (Δ > 0.5) | | |

TABLE 60
Radiographic Outcomes (mTSS) With and Without Concomitant
MTX Use

| | | Mean | Mean Change | P-Values |
|---|---|---|---|---|
| | | Baseline | in mTSS from | Between |
| | N | mTSS | Baseline | Groups |
| With MTX | | | | |
| Placebo (Week 24) | 73 | 27.4 | 1.0 | |
| Adalimumab (Week 48) | 73 | 24.1 | −0.1 | <0.001 |
| Without MTX | | | | |
| Placebo (Week 24) | 68 | 16.4 | 0.8 | |
| Adalimumab (Week 48) | 60 | 22.6 | 0.4 | 0.045 |

### Conclusions

Adalimumab was efficacious in inhibiting radiographic disease progression in PsA out to 48 weeks. Adalimumab inhibited radiographic disease progression whether or not MTX was being used at baseline.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

1. A method of determining the efficacy of a TNFα inhibitor for treating psoriatic arthritis in a subject comprising:
determining an ACR20 response of a patient population having psoriatic arthritis who was administered the TNFα inhibitor,
wherein an ACR20 response in at least about 39% of the patient population indicates that the TNFα inhibitor is an effective TNFα inhibitor for the treatment of psoriatic arthritis.

2. The method of claim 1, further comprising administering the effective TNFα inhibitor to a subject to treat psoriatic arthritis.

3-6. (canceled)

7. A method of treating psoriatic arthritis in a subject comprising administering an effective TNFα inhibitor to the subject such that psoriatic arthritis is treated, wherein the effective TNFα inhibitor was previously identified as achieving an ACR20 response in at least about 39% of a patient population having PsA.

8-30. (canceled)

31. A method of determining the efficacy of a TNFα inhibitor for treating psoriatic arthritis in a subject comprising:
determining a PASI50 response of a patient population having psoriatic arthritis who was administered the TNFα inhibitor,
wherein a PASI50 response in at least about 73% of the patient population indicates that the TNFα inhibitor is an effective TNFα inhibitor for the treatment of psoriatic arthritis in a subject.

32. The method of claim 31, further comprising administering the effective TNFα inhibitor to a subject to treat psoriatic arthritis.

33-51. (canceled)

52. A method of determining the efficacy of a TNFα inhibitor for treating psoriatic arthritis in a subject comprising:
determining a PGA response of “Clear” or “Almost Clear,” of a patient population having psoriatic arthritis who was administered the TNFα inhibitor,
wherein a PGA response of “Clear” or “Almost Clear,” in at least about 40% of the patient population indicates that the TNFα inhibitor is an effective TNFα inhibitor for the treatment of psoriatic arthritis in a subject.

53. The method of claim 52, further comprising administering the effective TNFα inhibitor to a subject to treat psoriatic arthritis.

54-60. (canceled)

61. A method of determining the efficacy of a TNFα inhibitor for treating psoriatic arthritis in a subject comprising:
determining a Health Assessment Questionnaire (HAQ) response of a patient population having psoriatic arthritis and who was administered the TNFα inhibitor,
wherein an average decrease of about 0.3 in the HAQ score of the patient population indicates that the TNFα inhibitor is an effective TNFα inhibitor for the treatment of psoriatic arthritis in a subject.

62. The method of claim 61, further comprising administering the effective TNFα inhibitor to a subject to treat psoriatic arthritis.

63-93. (canceled)


SEQUENCE LISTING (37 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  adalimumab light chain variable region CDR3
QRYNRAPYX

SEQ ID NO: 4 - protein, 12 aa, Artificial Sequence
  adalimumab heavy chain variable region CDR3
VSYLSTASSLDX

SEQ ID NO: 5 - protein, 7 aa, Artificial Sequence
  adalimumab light chain variable region CDR2
AASTLQS

SEQ ID NO: 6 - protein, 17 aa, Artificial Sequence
  adalimumab heavy chain variable region CDR2
AITWNSGHIDYADSVEG

SEQ ID NO: 7 - protein, 11 aa, Artificial Sequence
  adalimumab light chain variable region CDR1
RASQGIRNYLA

SEQ ID NO: 8 - protein, 5 aa, Artificial Sequence
  adalimumab heavy chain variable region CDR1
DYAMH

SEQ ID NO: 9 - protein, 107 aa, Artificial Sequence
  2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  2SD4 light chain variable region CDR3
QKYNSAPYA

SEQ ID NO: 12 - protein, 9 aa, Artificial Sequence
  EP B12  light chain variable region CDR3
QKYNRAPYA

SEQ ID NO: 13 - protein, 9 aa, Artificial Sequence
  VL10E4 light chain variable region CDR3
QKYQRAPYT

SEQ ID NO: 14 - protein, 9 aa, Artificial Sequence
  VL100A9 light chain variable region CDR3
QKYSSAPYT

SEQ ID NO: 15 - protein, 9 aa, Artificial Sequence
  VLL100D2 light chain variable region CDR3
QKYNSAPYT

SEQ ID NO: 16 - protein, 9 aa, Artificial Sequence
  VLL0F4 light chain variable region CDR3
QKYNRAPYT

SEQ ID NO: 17 - protein, 9 aa, Artificial Sequence
  LOE5 light chain variable region CDR3
QKYNSAPYY

SEQ ID NO: 18 - protein, 9 aa, Artificial Sequence
  VLLOG7 light chain variable region CDR3
QKYNSAPYN

SEQ ID NO: 19 - protein, 9 aa, Artificial Sequence
  VLLOG9 light chain variable region CDR3
QKYTSAPYT

SEQ ID NO: 20 - protein, 9 aa, Artificial Sequence
  VLLOH1 light chain variable region CDR3
QKYNRAPYN

SEQ ID NO: 21 - protein, 9 aa, Artificial Sequence
  VLLOH10 light chain variable region CDR3
QKYNSAAYS

SEQ ID NO: 22 - protein, 9 aa, Artificial Sequence
  VL1B7 light chain variable region CDR3
QQYNSAPDT

SEQ ID NO: 23 - protein, 9 aa, Artificial Sequence
  VL1C1 light chain variable region CDR3
QKYNSDPYT

SEQ ID NO: 24 - protein, 9 aa, Artificial Sequence
  VL0.1F4 light chain variable region CDR3
QKYISAPYT

SEQ ID NO: 25 - protein, 9 aa, Artificial Sequence
  VL0.1H8 light chain variable region CDR3
QKYNRPPYT

SEQ ID NO: 26 - protein, 9 aa, Artificial Sequence
  LOE7.A light chain variable region CDR3
QRYNRAPYA

SEQ ID NO: 27 - protein, 12 aa, Artificial Sequence
  2SD4 heavy chain variable region CDR3
ASYLSTSSSLDN

SEQ ID NO: 28 - protein, 12 aa, Artificial Sequence
  VH1B11 heavy chain variable region CDR3
ASYLSTSSSLDK

SEQ ID NO: 29 - protein, 12 aa, Artificial Sequence
  VH1D8 heavy chain variable region CDR3
ASYLSTSSSLDY

SEQ ID NO: 30 - protein, 12 aa, Artificial Sequence
  VH1A11 heavy chain variable region CDR3
ASYLSTSSSLDD

SEQ ID NO: 31 - protein, 12 aa, Artificial Sequence
  VH1B12 heavy chain variable region CDR3
ASYLSTSFSLDY

SEQ ID NO: 32 - protein, 12 aa, Artificial Sequence
  VH1E4 heavy chain variable region CDR3
ASYLSTSSSLHY

SEQ ID NO: 33 - protein, 12 aa, Artificial Sequence
  VH1F6 heavy chain variable region CDR3
ASFLSTSSSLEY

SEQ ID NO: 34 - protein, 12 aa, Artificial Sequence
  3C-H2 heavy chain variable region CDR3
ASYLSTASSLEY

SEQ ID NO: 35 - protein, 12 aa, Artificial Sequence
  VH1-D2.N heavy chain variable region CDR3
VSYLSTASSLDN

SEQ ID NO: 36 - DNA, 321 bp, Artificial Sequence
  adalimumab light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial Sequence
  adalimumab heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt